(19) United States
(12) Patent Application Publication
(10) Pub. No.: US 2009/0023742 A1
(43) Pub. Date: Jan. 22, 2009

(54) THIAZOLONES FOR USE AS PI3 KINASE INHIBITORS

(76) Inventors: Dashyant Dhanak, Collegeville, PA (US); Steven David Knight, Collegeville, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 12/281,179

(22) PCT Filed: Mar. 2, 2007
(86) PCT No.: PCT/US2007/063112

§ 371 Date: Aug. 29, 2008

Related U.S. Application Data
(60) Provisional application No. 60/778,428, filed on Mar. 2, 2006.

(51) Int. Cl. A61K 31/498; A61K 31/426; A61K 31/4439; A61K 31/433; A61K 31/4245; A61K 31/496; A61P 29/00; A61P 25/00; A61P 11/06; A61P 19/02; A61P 13/12; A61P 35/00; A61P 9/00; A61P 37/02; A61K 31/437; A61K 31/454; A61K 31/428; A61K 31/4709

(52) U.S. Cl. 514/249; 514/369; 514/314; 514/342; 514/367; 514/362; 514/326; 514/364; 514/300; 514/253.06

(57) ABSTRACT

Invented is a method of inhibiting the activity/function of PI3 kinases using substituted thiazolones. Also invented is a method of treating one or more disease states selected from: autoimmune disorders, inflammatory diseases, cardiovascular diseases, neurodegenerative diseases, allergy, asthma, pancreatitis, multiorgan failure, kidney diseases, platelet aggregation, cancer, sperm motility, transplantation rejection, graft rejection and lung injuries by the administration of substituted thiazolones.

### FIELD OF THE INVENTION

This invention relates to the use of substituted thiazolones for the modulation, notably the inhibition of the activity or function of the phosphorinositide-3′OH kinase family (hereinafter PI3 kinases), suitably, PI3Kα, PI3Kδ, PI3Kβ, and/or PI3Kγ. Suitably, the present invention relates to the use of substituted thiazolones in the treatment of one or more disease states selected from: autoimmune disorders, inflammatory diseases, cardiovascular diseases, neurodegenerative diseases, allergy, asthma, pancreatitis, multiorgan failure, kidney diseases, platelet aggregation, cancer, sperm motility, transplantation rejection, graft rejection and lung injuries.

### BACKGROUND OF THE INVENTION

Cellular plasma membranes can be viewed as a large store of second messenger that can be enlisted in a variety of signal transduction pathways. As regards function and regulation of effector enzymes in phospholipids signaling pathways, these enzymes generate second messengers from the membrane phospholipids pool (class I PI3 kinases (e.g. PI3 Kgamma)) are dual-specific kinase enzymes, means they display both: lipid kinase (phosphorylation of phosphorinositides) as well as protein kinase activity, shown to be capable of phosphorylation of other protein as substrates, including auto-phosphorylation as intramolecular regulatory mechanism. These enzymes of phospholipids signaling are activated in response to a variety of extra-cellular signals such as growth factors, mitogens, integrins (cell-cell interactions) hormones, cytokines, viruses and neurotransmitters such as described in Scheme 1 hereinafter and also by intra-cellular cross regulation by other signaling molecules (cross-talk, where the original signal can activate some parallel pathways that in a second step transmit signals to PI3Ks by intra-cellular signaling events), such as small GTPases, kinases or phosphatases for example. The inositol phospholipids (phosphoinositides) intracellular signaling pathway begins with binding of a signaling molecule (extra cellular ligands, stimuli, receptor dimerization, transactivation by heterologous receptor (e.g. receptor tyrosine kinase)) to a G-protein linked transmembrane receptor integrated into the plasma membrane.

PI3K converts the membrane phospholipids PIP(4,5)2 into PIP(3,4,5)3 which in turn can be further converted into another 3′ phosphorylated form of phosphoinositides by 5′-specific phosphor-inositide phophatases, thus PI3K enzymatic activity results either directly or indirectly in the generation of two 3′-phosphoinositide subtypes that function as 2^{nd} messengers in intr-cellular signal transduction (Trends Biochem. Sci. 22(7) p. 267-72 (1997) by Vanhaesebroeck et al.: Chem. Rev. 101(8) p. 2365-80 (2001) by Leslie et al (2001); Annu. Rev. Cell. Dev. Biol. 17p, 615-75 (2001) by Katso et al. and Cell. Mol. Life. Sci. 59(5) p. 761-79 (2002) by Toker et al.). Multiple PI3K insoforms categorized by their catalytic subunits, their regulation by corresponding regulatory subunits, expression patterns and signaling-specific functions (p110α, β, and γ) perform this enzymatic reaction (Exp. Cell. Res. 25 (1) p. 239-54 (1999) by Vanhaesebroeck and Katso et al., 2001, above).

The evolutionary conserved insoforms p110α and β are ubiquitously express, which δ and γ are more specifically expressed in the haematopoietic cell system, smooth muscle cells, myocytes and endothelial cells (Trends Biochem. Sci. 22(7) p. 267-72 (1997) by Vanhaesebroeck et al.). Their expression might also be regulated in an inducible manner depending on the cellular, tissue type and stimuli as well as disease context.

To date, eight mammalian PI3Ks have been identified, divided into three main classes (I, II, and III) on the basis of sequence homology, structure, binding partners, mode of activation, and substrate preference in vitro. Class I PI3Ks can phosphorylate phosphatidylinositol (PI), phosphatidylinositol-4-phosphate,m and phosphatidylinositol-4,5-biphosphate (PIP2) to produce phosphatidylinositol-3-phosphate (PIP), phosphatidylinositol-3,4-biphosphate, and phosphatidylinositol-3,4,5-triphosphate, respectively. Class II PI3Ks phosphorylate PI and phosphatidylinositol-4-phosphate. Class III PI3Ks can only phosphorylate PI (Vanhaesebrokeck et al., 1997, above; Vanhaesebroeck et al., 1999, above and Leslie et al, 2001, above) G-protein coupled receptors mediated phosphoinositide 3′OH-kinase activation via small GTPases such as Gβγ and Ras, and consequently PI3K signaling plays a central role in establishing and coordinating cell polarity and dynamic organization of the cytoskeleton—which together provides the driving force of cells to move.

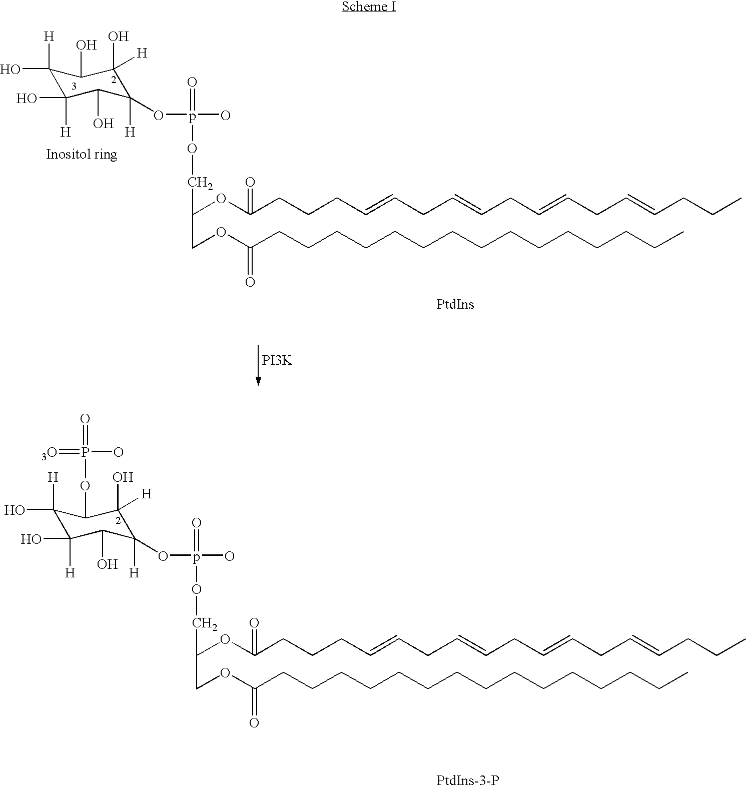

As illustrated in Scheme 1 above, Phosphoinositide 3-kinase (PI3K) is involved in the phosphorylation of Phosphatidylinositol (PtdIns) on the third carbon of the inositol ring. The phosphorylation of PtdIns to 3,4,5-triphosphate (PtdIns(3,4,5)P₃), PtdIns(3,4)P₂ and PtdIns(3)P acts as second messengers for a variety of signal transduction pathways, including those essential to cell proliferation, cell differentiation, cell growth, cell size, cell survival, apoptosis, adhesion, cell motility, cell migration, chemotaxis, invasion, cytoskeletal rearrangement, cell shape changes, vesicle trafficking and metabolic pathway (Katso et al., 2001, above and Mol. Med. Today 6(9) p. 347-57 (2000) by Stein). Chemotaxis—the directed movement of cells toward a concentration gradient of chemical attractants, also called chemokines is involved in many important diseases such as inflammation/auto-immunity, neurodegeneration, antiogenesis, invasion/metastasis and wound healing (Immunol. Today 21(6) p. 260-4 (2000) by Wyman et al.; Science 287(5455) p. 1049-53 (2000) by Hirsch et al.; FASEB J. 15(11) p. 2019-21 (2001) by Hirsch et al. and Nat. Immunol. 2(2) p. 108-15 (2001) by Gerard et al.).

Recent advances using genetic approaches and pharmacological tools have provided insights into signalling and molecular pathways that mediate chemotaxis in response to chemoattractant activated G-protein coupled receptors PI3-Kinase, responsible for generating these phosphorylated signalling products, was originally identified as an activity associated with viral oncoproteins and growth factor receptor tyrosine kinases that phosphorylates phosphatidylinositol (PI) and its phosphorylated derivatives at the 3′-hydroxyl of the inositol ring (Panayotou et al., Trends Cell Biol. 2 p. 358-60 (1992)). However, more recent biochemical studies revealed that, class I PI3 kinases (e.g. class IB isoform PI3Kγ) are dual-specific kinase enzymes, means they display both: lipid kinase (phosphorylation of phospho-inositides) as well as protein kinase activity, shown to be capable of phosphorylation of other protein as substrates, including auto-phosphorylation as intra-molecular regulatory mechanism.

PI3-kinase activation, is therefore believe to be involved in a range of cellular responses including cell growth, differentiation, and apoptosis (Parker et al., Current Biology, 5 p. 577-99 (1995); Yao et al., Science, 267 p. 2003-05 (1995)). PI3-kinase appears to be involved in a number of aspects of leukocyte activation. A p85-associated PI3-kinase activity has been shown to physically associate with the cytoplasmic domain of CD28, which is an important costimulatory molecule for the activation of T-cells in response to antigen (Pages et al., Nature, 369 p. 327-29 (1994); Rudd, Immunity 4 p. 527-34 (1996)). Activation of T cells through CD28 lowers the threshold for activation by antigen and increases the magnitude and duration of the proliferative response. These effects are linked to increases in the transcription of a number of genes including interleukin-2 (IL2), an important T cell growth factor (Fraser et al., Science 251 p. 313-16 (1991)). Mutation of CD28 such that it can longer interact with PI3-kinase leads to a failure to initiate IL2 production, suggesting a critical role for PI3-kinase in T cell activation. PI3Kγ has been identified as a mediator of G beta-gamma-dependent regulation of JNK activity, and G beta-gamma are subunits of heterotrimeric G proteins (Lopez-Ilasaca et al., J. Biol. Chem. 273(5) p. 2505-8 (1998)). Cellular processes in which PI3Ks play an essential role include suppression of apoptosis, reorganization of the actin skeleton, cardiac myocyte growth, glycogen synthase stimulation by insulin, TNFα-mediated neutrophil priming and superoxide generation, and leukocyte migration and adhesion to endothelial cells.

Recently, (Laffargue et al., Immunity 16(3) p. 441-51 (2002)) it has been described that PI3Kγ relays inflammatory signals through various G(i)-coupled receptors and its central to mast cell function, stimuli in context of leukocytes, immunology includes cytokines, chemokines, adenosines, antibodies, integrins, aggregation factors, growth factors, viruses or hormones for example (J. Cell. Sci. 114(Pt 16) p. 2903-10 (2001) by Lawlor et al.; Laffargue et al., 2002, above and Curr. Opinion Cell Biol. 14(2) p. 203-13 (2002) by Stephens et al.).

Specific inhibitors against individual members of a family of enzymes provide invaluable tools for deciphering functions of each enzyme. Two compounds, LY294002 and wortmannin (cf. hereinafter), have been widely used as PI3-kinase inhibitors. These compounds are non-specific PI3K inhibitors, as they do not distinguish among the four members of Class I PI3-kinases. For example, the IC₅₀ values of wortmannin against each of the various Class I PI3-kinases are in the range of 1-10 nM. Similarly, the IC₅₀ values for LY294002 against each of these PI3-kinases is about 15-20 μM (Fruman et al., Ann. Rev. Biochem., 67, p. 481-507 (1998)), also 5-10 microM on CK2 protein kinase and some inhibitory activity on phospholipases. Wortmannin is a fungal metabolite which irreversibly inhibits PI3K activity by binding covalently to the catalytic domain of this enzyme. Inhibition of PI3K activity by wortmannin eliminates subsequent cellular response to the extracellular factor. For example, neutrophils respond to the chemokine fMet-Leu-Phe (fMLP) by stimulating PI3K and synthesizing PtdIns (3, 4, 5)P₃. This synthesis correlates with activation of the respirators burst involved in neutrophil destruction of invading microorganisms. Treatment of neutrophils with wortmannin prevents the fMLP-induced respiratory burst response (Thelen et al., Proc. Natl. Acad. Sci. USA, 91, p. 4960-64 (1994)). Indeed, these experiments with wortmannin, as well as other experimental evidence, shows that PI3K activity in cells of hematopoietic lineage, particularly neutrophils, monocytes, and other types of leukocytes, is involved in many of the non-memory immune response associated with acute and chronic inflammation.

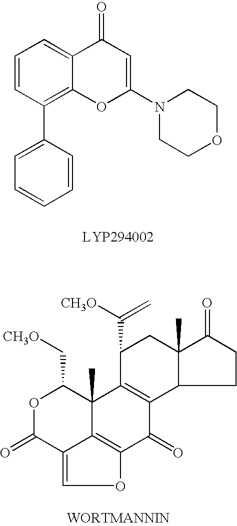

Based on studies using wortmannin, there is evidence that PI3-kinase function is also required for some aspects of leukocyte signaling through G-protein coupled receptors (Thelen et al., 1994, above). Moreover, it has been shown that wortmannin and LY294002 block neutrophil migration and superoxide release. Cyclooxygenase inhibiting benzofuran derivatives are disclosed by John M. Janusz et al., in J. Med. Chem. 1998; Vol. 41, No. 18.

It is now well understood that deregulation of onocogenes and tumour-suppressor genes contributes to the formation fo malignant tumours, for example by way of increase cell proliferation or increased cell survival. It is also now known that signaling pathways mediated by the PI3k family have a central role in a number of cell processes including proliferation and survival, and deregulation of these pathways is a causative factor a wide spectrum of human cancers and other diseases (Katso et al., Annual Rev. Cell Dev. Biol. 2001, 17: 615-617 and Foster et al., J. Cell Science, 2003, 116: 3037-3040).

Class I PI3K is a heterodimer consisting of a p110 catalytic subunit and a regulatory subunit, and the family is further divided into class Ia and Class Ib enzymes on the basis of regulatory partners and mechanism of regulation. Class Ia enzymes consist of three distinct catalytic subunits (p110α, p110β, and p110δ) that dimerise with five distinct regulatory subunits (p85α, p55α, p50α, p85β, and p55γ), with all catalytic subunits being able to interact with all regulatory subunits to form a variety of heterodimers. Class Ia PI3K are generally activated in response to growth factor-stimulation of receptor tyrosine kinases, via interaction of the regulatory subunit SH2 domains with specific phosphor-tyrosine residues of the activated receptor or adaptor proteins such as IRS-1. Both p110α and p110β are constitutively expressed in all cell types, whereas p110δ expression is more restricted to leukocyte populations and some epithelial cells. In contrast, the single Class Ib enzyme consists of a p110γ catalytic subunit that interacts with a p101 regulatory subunit. Furthermore, the Class Ib enzyme is activated in response to G-protein coupled receptor (GPCR) systems and its expression appears to be limited to leucoccytes.

There is now considerable evidence indicating that Class Ia PI3K enzymes contribute to tumourigenesis in a wide variety of human cancers, either directly or indirectly (Vivanco and Sawyers, Nature Reviews Cancer, 2002, 2, 489-501). For example, the p110α subunit is amplified in some tumours such as those of the ovary (Shayesteh, et al., Nature Genetics, 1999, 21: 99-102) and cervix (Ma et al., Oncogene, 2000, 19: 2739-2744). More recently, activating mutations within p110α have been associated with various other tumors such as those of the colorectal region and of the breast and lung (Samuels, et al., Science, 2004, 304, 554). Tumor-related mutations in p85α have also been identified in cancers such as those of the ovary and colon (Philp et al., Cancer Research, 2001, 61, 7426-7429). In addition to direct effects, it is believed that activation of Class Ia PI3K contributes to tumourigenic events that occur upstream in signaling pathways, for example by way of ligan-dependent or ligand-independent activation of receptor tyrosine kinases, GPCR systems or integrins (Vara et al., Cancer Treatment Reviews, 2004, 30, 193-204). Examples of such upstream signaling pathways include over-expression of the receptor tyrosine kinase Erb2 in a variety of tumors leading to activation of PI3K-mediated pathways (Harari et al., Oncogene, 2000, 19, 6102-6114) and over-expression of the oncogene Ras (Kauffmann-Zeh et al., Nature, 1997, 385, 544-548). In addition, Class Ia PI3Ks may contribute indirectly to tumourigenesis caused by various downstream signaling events. For example, loss of the effect of the PTEN tumor-suppressor phosphatase that catalyses conversion of PI(3,4,5)P3 back to PI(4,5)P2 is associated with a very broad range of tumors via deregulation of PI3K-mediated production of PI(3,4,5)P3 (Simpson and Parsons, Exp. Cell Res., 2001, 264, 29-41). Furthermore, augmentation of the effects of other PI3K-mediated signaling events is believed to contribute to a variety of cancers, for example by activation of AKT (Nicholson and Andeson, Cellular Signaling, 2002, 14, 381-395).

In addition to a role in mediating proliferative and survival signaling in tumor cells, ther is also good evidence that class Ia PI3K enzymes will also contribute to tumourigenesis via its function in tumor-associated stromal cells. For examples, PI3K signaling is known to play an important role in mediating angiogenic events in endothelial cells in response to pro-angiogenic factors such as VEGF (abid et al., Arterioscler, Thromb. Vasc. Biol., 2004, 24, 294-300). As Class I PI3K enzymes are also involved in motility and migration (Sawyer, Expert Opinion investing. Drugs, 2004, 13, 1-19), PI3K inhibitors should provide therapeutic benefit via inhibition of tumor cell invasion and metastasis.

### DESCRIPTION OF THE RELATED ART

International application No. PCT/US01/37658, filed Nov. 18, 2003, the entrie disclosure of which is hereby incorporated by reference, describes a group of thiazolidinone compounds which are indicated as having hYAK3 inhibitory activity and which are indicated as being useful in the treatment of deficiencies in hematopoietic cells, in particular in the treatment of deficiencies in erythroid cells.

International application No. PCT/US01/37658 does not disclose the use of any of the compounds described therein as inhibitors or inhibitors of PI3 kinases.

### SUMMARY OF THE INVENTION

This invention relates to a method of inhibiting one or more PI3 kinases selected from: PI3Kα, PI3Kδ, PI3Kβ and PI3Kγ, in a mammal in need thereof, which method comprises administrating to such mammal a therapeutically effective amount of a compound of Formula (I):

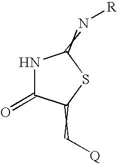

in which
R is C₃₋₆ cycloalkyl or naphtyl; or
R is

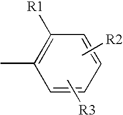

in which R1 is hydrogen, halogen, —C₁₋₆alkyl, —SC₁₋₆alkyl, —OC₁₋₆alkyl, —NO₂,
—S(═O)—C₁₋₆alkyl, —OH, —CF₃, —CN, —CO₂H, —OCF₃, or —CO₂C₁₋₆alkyl;
and R2 and R3 are independently hydrogen, halogen, —C₁₋₆ alkyl, —SC₁₋₆alkyl, —OC₁₋₆alkyl, —NO₂, —S(═O)—C₁₋₆alkyl, —OH, —CF₃, —CN, —CO₂H, —CO₂C₁₋₆alkyl, —CONH₂, —NH₂, —OCH₂(C═O)OH, —OCH₂CH₂OCH₃, —SO₂NH₂,
—CH₂SO₂CH₃, —NH(C═NH)CH₃; or R2 and R3 can independently be a radical of the formula

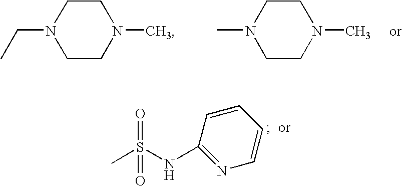

R is

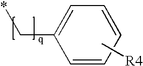

in which q is one or two; R4 is hydrogen, halogen, or —SO₂NH₂; or
R is —(CH₂)ₙ—NR^{k}R^{l} in which n is 2 or 3, and R^{k} and R^{l} are independently —C₁₋₆alkyl; or —NR^{k}R^{l} together form

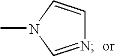

R is

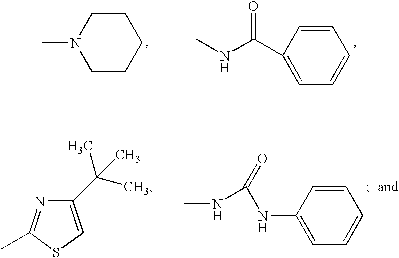

Q is

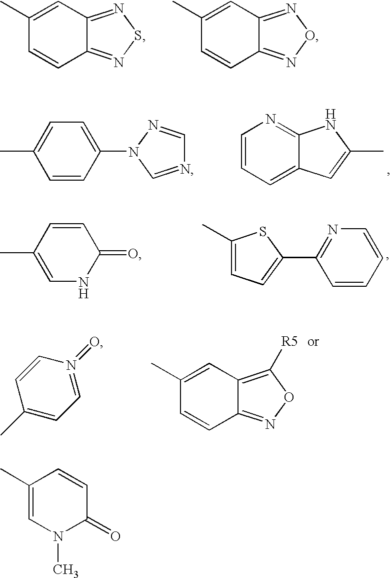

in which R5 is hydrogen, phenyl optionally substituted with up to three C₁₋₆ alkyl or halogen, or C₁₋₆ alkyl; or
Q is

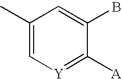

in which Y is CH; and A and B together are a part of

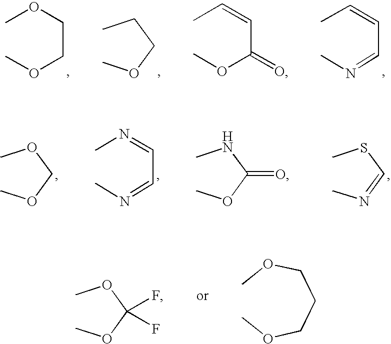

provided that ortho position to Y is N or O; or
Q is

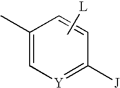

in which Y is N or CH; J is hydrogen, NH₂, OH or —OC₁₋₆alkyl; and L is hydrogen, NH₂, halogen, —NO₂, or —OC₁₋₆alkyl,
   and/or a pharmaceutically acceptable salt, hydrate, solvate or pro-drug thereof.

This invention also relates to a method of treating cancer, which comprises administering to a subject in need thereof an effective amount of a compound of Formula (I).

This invention also relates to a method of treating one or more disease states selected from: autoimmune disorders, inflammatory diseases, cardiovascular diseases, neurodegenerative diseases, allergy, asthma, pancreatitis, multiorgan failure, kidney diseases, platelet aggregation, sperm motility, transplantation rejection, graft rejection and lung injuries, which comprises administering to a subject in need thereof an effective amount of a compound of Formula (I).

Included in the present invention are methods of co-administering the present PI3 kinase inhibiting compounds with further active ingredients.

### DETAILED DESCRIPTION OF THE INVENTION

The present invention relates to compounds of Formula (I) described above as PI3 kinase inhibitors. Suitably, the compounds of Formula (I) inhibit one or more PI3 kinases selected from: PI3Kα, PI3Kδ, PI3Kβ and PI3Kγ.

The invention also relates to a compound of the formula II,

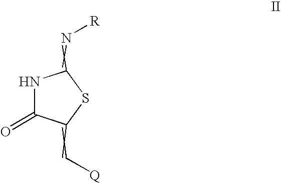

in which
R is C₃₋₆ cycloalkyl or naphtyl; or
R is

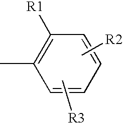

in which R1 is hydrogen, halogen, —C₁₋₆alkyl, —SC₁₋₆alkyl, —OC₁₋₆alkyl, —NO₂,
—S(═O)—C₁₋₆alkyl, —OH, —CF₃, —CN, —CO₂H, —OCF₃, or —CO₂C₁₋₆alkyl;
and R2 and R3 are independently hydrogen, halogen, —C₁₋₆ alkyl, —SC₁₋₆alkyl, —OC₁₋₆alkyl, —NO₂, —S(═O)—C₁₋₆alkyl, —OH, —CF₃, —CN, —CO₂H, —CO₂C₁₋₆alkyl, —CONH₂, —NH₂, —OCH₂(C═O)OH, —OCH₂CH₂OCH₃, —SO₂NH₂,
—CH₂SO₂CH₃, —NH(C═NH)CH₃; or R2 and R3 can independently be a radical of the formula

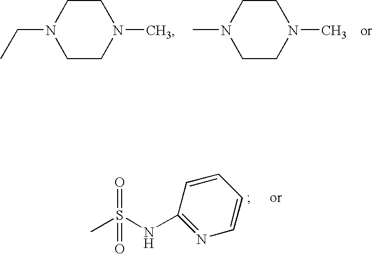

R is

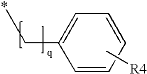

in which q is one or two; R4 is hydrogen, halogen, or —SO₂NH₂; or
R is —(CH₂)ₙ—NR^{k}R^{l} in which n is 2 or 3, and R^{k} and R^{l} are independently —C₁₋₆alkyl; or —NR^{k}R^{l} together form

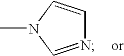

R is

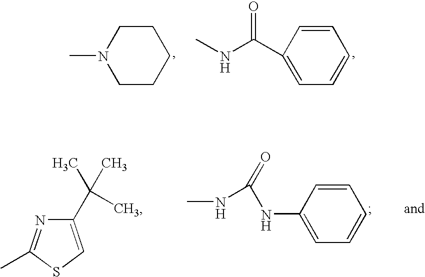

Q is

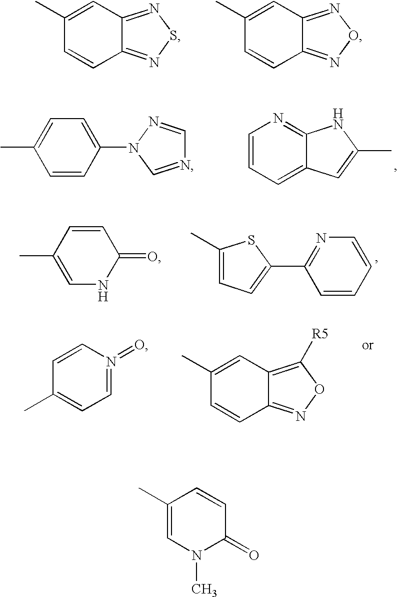

in which R5 is hydrogen, phenyl optionally substituted with up to three C₁₋₆ alkyl or halogen, or C₁₋₆ alkyl; or
Q is

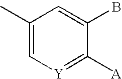

in which Y is CH; and A and B together are a part of

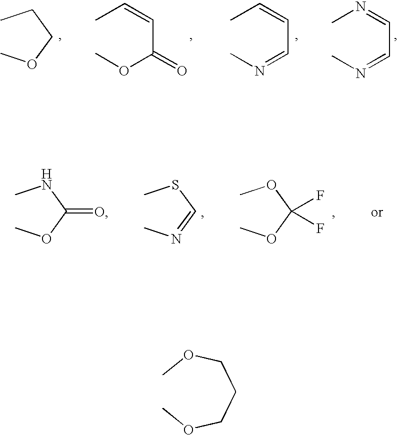

provided that ortho position to Y is N or O,
   and/or a pharmaceutically acceptable salt, hydrate, solvate or pro-drug thereof.

In one embodiment, in a compound of formula I or II
R is C₃₋₆ cycloalkyl or naphtyl; or
R is

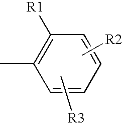

in which R1 is hydrogen, halogen, —C₁₋₆alkyl, —SC₁₋₆alkyl, —OC₁₋₆alkyl, —NO₂,
—S(═O)—C₁₋₆alkyl, —OH, —CF₃, —CN, —CO₂H, —OCF₃, or —CO₂C₁₋₆alkyl;
and R2 and R3 are independently hydrogen, halogen, —C₁₋₆ alkyl, —SC₁₋₆alkyl, —OC₁₋₆alkyl, —NO₂, —S(═O)—C₁₋₆alkyl, —OH, —CF₃, —CN, —CO₂H, —CO₂C₁₋₆alkyl, —CONH₂, —NH₂, —OCH₂(C═O)OH, —OCH₂CH₂OCH₃, —SO₂NH₂,
—CH₂SO₂CH₃, —NH(C═NH)CH₃; or R2 and R3 can independently be a radical of the formula

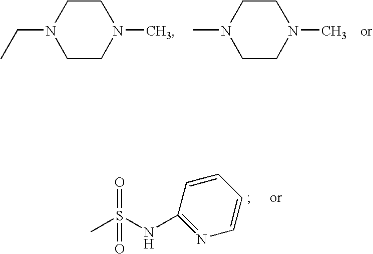

R is

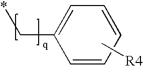

in which q is one or two; R4 is hydrogen, halogen, or —SO₂NH₂; or
R is —(CH₂)ₙ—NR^{k}R^{l} in which n is 2 or 3, and R^{k} and R^{l} are independently —C₁₋₆alkyl; or —NR^{k}R^{l} together form

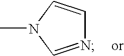

R is

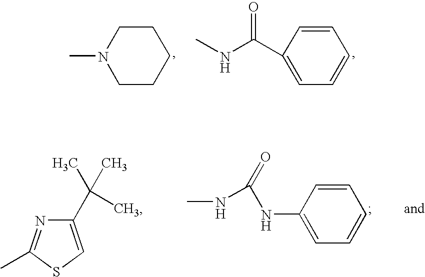

Q is

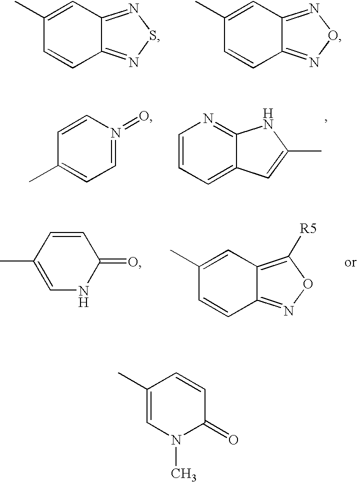

in which R5 is hydrogen, phenyl optionally substituted with up to three C₁₋₆ alkyl or halogen, or C₁₋₆ alkyl; or
Q is

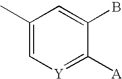

in which Y is CH; and A and B together are a part of

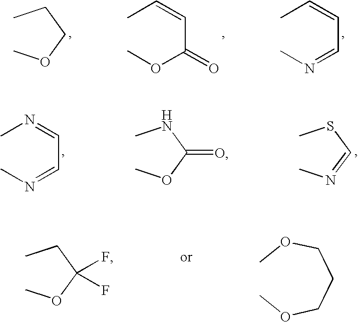

provided that ortho position to Y is N or O.

In another embodiment, R radical of compounds of formula I and II are

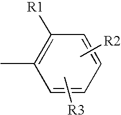

in which R1 is halogen, —C₁₋₆alkyl, —SC₁₋₆alkyl, —OC₁₋₆alkyl, —NO₂, —S(═O)—C₁₋₆alkyl, —OH, —CF₃, —CN, —CO₂H, or —CO₂C₁₋₆alkyl;
and R2 and R3 are independently hydrogen, halogen, —C₁₋₆ alkyl, —SC₁₋₆alkyl, —OC₁₋₆alkyl, —NO₂, —S(═O)—C₁₋₆alkyl, —OH, —CF₃, —CN, —CO₂H, —CO₂C₁₋₆alkyl, —NH₂, or —NH(C═NH)CH₃;
and
Q is

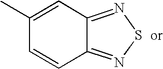

Q is

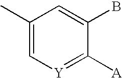

in which Y is CH; and A and B together are a part of

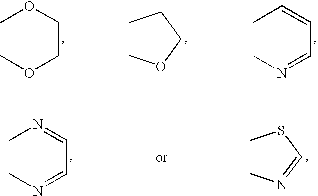

provided that ortho position to Y is N or O.

Yet in another one embodiment, in formula I or II, R is

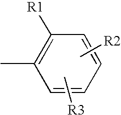

in which R1 is halogen, —C₁₋₆alkyl, —SC₁₋₆alkyl, —OC₁₋₆alkyl, —NO₂, —S(═O)—C₁₋₆alkyl, —OH, —CF₃, —CN, —CO₂H, or —CO₂C₁₋₆alkyl;
and R2 and R3 are independently hydrogen, halogen, —C₁₋₆ alkyl, —SC₁₋₆alkyl, —OC₁₋₆alkyl, —NO₂, —S(═O)—C₁₋₆alkyl, —OH, —CF₃, —CN, —CO₂H, —CO₂C₁₋₆alkyl, —NH₂, or —NH(C═NH)CH₃;
and
Q is

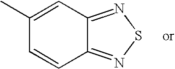

Q is

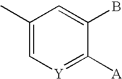

in which Y is CH; and A and B together are a part of

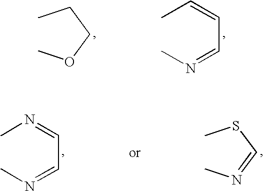

provided that ortho position to Y is N or O.

Yet in a further embodiment, in a compound of formula I or II,
R is

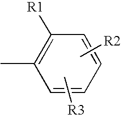

in which R1 is halogen, —C₁₋₆alkyl, —SC₁₋₆alkyl, —OC₁₋₆alkyl, —NO₂, —S(═O)—C₁₋₆alkyl, —OH, —CF₃, —CN, —CO₂H, or —CO₂C₁₋₆alkyl;
and R2 and R3 are independently hydrogen, halogen, —C₁₋₆ alkyl, —SC₁₋₆alkyl, —OC₁₋₆alkyl, —NO₂, —S(═O)—C₁₋₆alkyl, —OH, —CF₃, —CN, —CO₂H, —CO₂C₁₋₆alkyl, —NH₂, or —NH(C═NH)CH₃;
and
Q is

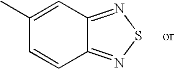

Q is

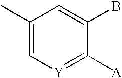

in which Y is CH; and A and B together are a part of

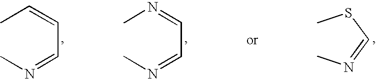

provided that ortho position to Y is N.

The invention also relates to compounds selected from:
2-(2-Chloro-5-fluoro-phenylimino)-5-(2,3-dihydro-benzo[1-6]dioxin-6-ylmethylene)-thiazolidin-4-one;
2-(2-Chloro-phenylimino)-5-(2-oxo-2H-chromen-6-ylmethylene)-thiazolidin-4-one;
2-(2-Chloro-phenylimino)-5-(2-oxo-2H-chromen-6-ylmethylene)-thiazolidin-4-one;
2-(2-Chloro-phenylimino)-5-(2-oxo-2H-chromen-6-ylmethylene)-thiazolidin-4-one;
5-(2,3-Dihydro-benzofuran-5-ylmethylene)-2-(2,4,6-trimethyl-phenylimino)-thiazolidin-4-one;
2-Cyclohexylimino-5-(2,3-dihydro-benzo[1-6]dioxin-6-ylmethylene)-thiazolidin-4-one;
2-Cyclohexylimino-5-(2,3-dihydro-benzofuran-5-ylmethylene)-thiazolidin-4-one;
5-(2,3-Dihydro-benzofuran-5-ylmethylene)-2-o-tolylimino-thiazolidin-4-one;
5-(2,3-Dihydro-benzo[1-6]dioxin-6-ylmethylene)-2-o-tolylimino-thiazolidin-4-one;
5-[2-(2-Chloro-phenylimino)-4-oxo-thiazolidin-5-ylidenemethyl]-3H-benzooxazol-2-one;
2-(2-Trifluoromethyl-phenylimino)-5-(2,3-dihydro-benzofuran-5-ylmethylene)-thiazolidin-4-one;
2-(2-Bromo-phenylimino)-5-(2,3-dihydro-benzofuran-5-ylmethylene)-thiazolidin-4-one;
2-(2,6-Dichloro-phenylimino)-5-(2,3-dihydro-benzofuran-5-ylmethylene)-thiazolidin-4-one;
5-(2,3-Dihydro-benzofuran-5-ylmethylene)-2-(2-methylsulfanyl-phenylimino)-thiazolidin-4-one;
5-(2,3-Dihydro-benzofuran-5-ylmethylene)-2-(2-fluoro-phenylimino)-thiazolidin-4-one;
2-(2-Methylsulfanyl-phenylimino)-5-(quinolin-6-ylmethylene)-thiazolidin-4-one;
2-(2-Bromo-phenylimino)-5-(quinolin-6-ylmethylene)-thiazolidin-4-one;
2-(2,3-Dimethyl-phenylimino)-5-(quinolin-6-ylmethylene)-thiazolidin-4-one;
2-(Naphthalen-1-ylimino)-5-(quinolin-6-ylmethylene)-thiazolidin-4-one;
5-(Quinolin-6-ylmethylene)-2-(2-trifluoromethyl-phenylimino)-thiazolidin-4-one;
2-(2-Chloro-5-trifluoromethyl-phenylimino)-5-(quinolin-6-ylmethylene)-thiazolidin-4-one;
2-(2,6-Dichloro-phenylimino)-5-8quinolin-6-ylmethylene)-thiazolidin-4-one;
2-(2-Bromo-phenylimino)-5-(2,3-dihydro-benzo[1-6]dioxin-6-ylmethylene)-thiazolidin-4-one;
2-(2-Chloro-phenylimino)-5-(quinoxalin-6-ylmethylene)-thiazolidin-4-one;
2-(2,6-Dichloro-phenylimino)-5-(2,3-dihydro-benzo[1-6]dioxin-6-ylmethylene)-thiazolidin-4-one;
5-(2,3-Dihydro-benzo[1-6]dioxin-6-ylmethylene)-2-(2-nitro-phenylimino)-thiazolidin-4-one;
5-(2,3-Dihydro-benzofuran-5-ylmethylene)-2-(2-nitro-phenylimino)-thiazolidin-4-one;
2-(2-Chloro-4-fluoro-5-methyl-phenylimino)-5-(2,3-dihydro-benzofuran-5-ylmethylene)-thiazolidin-4-one;
3-Chloro-4-[5-(2,3-dihydro-benzofuran-5-ylmethylene)-4-oxo-thiazolidin-2-ylideneamino]-benzoic acid methyl ester;
2-(2-Chloro-5-fluoro-phenylimino)-5-(2,3-dihydro-benzofuran-5-ylmethylene)-thiazolidin-4-one;
2-(2-Chloro-4-trifluoromethyl-phenylimino)-5-(2,3-dihydro-benzofuran-5-ylmethylene)-thiazolidin-4-one;
2-(4-Bromo-2-chloro-phenylimino)-5-(2,3-dihydro-benzofuran-5-ylmethylene)-thiazolidin-4-one;
5-(2,3-Dihydro-benzofuran-5-ylmethylene)-2-(2-methanesulfinyl-phenylimino)-thiazolidin-4-one;
3-Chloro-4-[5-(2,3-dihydro-benzofuran-5-ylmethylene)-4-oxo-thiazolidin-2-ylideneamino]-benzoic acid;
5-[2-(2-Chloro-phenylimino)-4-oxo-thiazolidin-5-ylidenemethyl]-1H-pyridin-2-one;
2-(2-Methylsulfanyl-phenylimino)-5-(quinolin-6-ylmethylene)-thiazolidin-4-one;
2-(2-Chloro-4-fluoro-5-methyl-phenylimino)-5-(quinolin-6-ylmethylene)-thiazolidin-4-one;
2-(2-Chloro-5-fluoro-phenylimino)-5-(quinolin-6-ylmethylene)-thiazolidin-4-one;
2-(2-Chloro-5-fluoro-phenylimino)-5-(2,3-dihydro-benzo[1-6]dioxin-6-ylmethylene)-thiazolidin-4-one;
2-(2-Chloro-4-trifluoromethyl-phenylimino)-5-quinolin-6-ylmethylene-thiazolidin-4-one;
5-(Benzothiazol-6-ylmethylene)-2-(2-chloro-phenylimino)-thiazolidin-4-one;
5-(Benzo[1,2,5]thiadiazol-5-ylmethylene)-2-(2-bromo-phenylimino)-thiazolidin-4-one;
5-(Benzo[1,2,5]thiadiazol-5-ylmethylene)-2-(2-chloro-5-fluoro-phenylimino)-thiazolidin-4-one;
5-(Benzothiazol-6-ylmethylene)-2-(2,6-dichloro-phenylimino)-thiazolidin-4-one;
2-(2-Chloro-phenylimino)-5-(4-hydroxy-3-nitro-benzylidene)-thiazolidin-4-one;
2-(2-Chloro-phenylimino)-5-(4-hydroxy-3-methoxy-benzylidene)-thiazolidin-4-one;
2-(2-Chloro-phenylimino)-5-(4-hydroxy-benzylidene)-thiazolidin-4-one;
2-(2-Chloro-phenylimino)-5-(4-methoxy-benzylidene)-thiazolidin-4-one;
5-(3-Chloro-4-hydroxy-benzylidene)-2-(2-chloro-phenylimino)-thiazolidin-4-one;
2-(2-Chloro-phenylimino)-5-(3-fluoro-4-methoxy-benzylidene)-thiazolidin-4-one;
2-(2,6-Dichloro-phenylimino)-5-(3-fluoro-4-hydroxy-benzylidene)-thiazolidin-4-one;
2-(2-Chloro-phenylimino)-5-(3-fluoro-4-hydroxy-benzylidene)-thiazolidin-4-one;
2-(2-Chloro-5-fluoro-phenylimino)-5-(3-fluoro-4-hydroxy-benzylidene)-thiazolidin-4-one;
5-(3-Fluoro-4-hydroxy-benzylidene)-2-o-tolylimino-thiazolidin-4-one;
2-(2-Chloro-phenylimino)-5-quinolin-6-ylmethylene-thiazolidin-4-one;
5-Quinolin-6-ylmethylene-2-(2,4,6-trimethyl-phenylimino)-thiazolidin-4-one;
5-Quinolin-6-ylmethylene-2-o-tolylimino-thiazolidin-4-one;
2-(2-Methoxy-phenylimino)-5-quinolin-6-ylmethylene-thiazolidin-4-one;
5-(2,3-Dihydro-benzofuran-5-ylmethylene)-2-(2-dimethylamino-ethylamino)-thiazol-4-one;
Benzoic acid N′-(4-oxo-5-quinolin-6-ylmethylene-4,5-dihydro-thiazol-2-yl)-hydrazide;
2-(2-Dimethylamino-ethylimino)-5-quinolin-6-ylmethylene-thiazolidin-4-one;
5-(2,3-Dihydro-benzofuran-5-ylmethylene)-2-(piperidin-1-ylamino)-thiazol-4-one;
2-Benzylamino-5-(2,3-dihydro-benzofuran-5-ylmethylene)-thiazol-4-one;
2-(4-tert-Butyl-thiazol-2-ylamino)-5-(2,3-dihydro-benzofuran-5-ylmethylene)-thiazol-4-one;
4-{[5-(2,3-Dihydro-benzofuran-5-ylmethylene)-4-oxo-4,5-dihydro-thiazol-2-ylamino]-methyl}-benzenesulfonamide;
5-(2,3-Dihydro-benzofuran-5-ylmethylene)-2-(3-dimethylamino-propylamino)-thiazol-4-one;
5-(2,3-Dihydro-benzofuran-5-ylmethylene)-2-(3-imidazol-1-yl-propylamino)-thiazol-4-one;
Phenyl-carbamic acid N′-[5-(2,3-dihydro-benzofuran-5-ylmethylene)-4-oxo-4,5-dihydro-thiazol-2-yl]-hydrazide;
Benzoic acid N′-[5-(2,3-dihydro-benzofuran-5-ylmethylene)-4-oxo-4,5-dihydro-thiazol-2-yl]-hydrazide;
5-Benzo[1,2,5]thiadiazol-5-ylmethylene-2-(2,3,4-trifluoro-phenylamino)-thiazol-4-one;
5-Benzo[1,2,5]oxadiazol-5-ylmethylene-2-(2-nitro-phenylamino)-thiazol-4-one;
2-(2,6-Dichloro-phenylamino)-5-(4-[1,2,4]triazol-1-yl-benzylidene)-thiazol-4-one;
2-(2,6-Dichloro-phenylamino)-5-(1H-pyrrolo[2,3-b]pyridin-2-ylmethylene)-thiazol-4-one;
5-Benzo[1,2,5]thiadiazol-5-ylmethylene-2-(2,6-dichloro-phenylamino)-thiazol-4-one;
5-[2-(2-Methoxy-6-methyl-phenylamino)-4-oxo-4H-thiazol-5-ylidenemethyl]-1H-pyridin-2-one;
5-Benzo[1,2,5]thiadiazol-5-ylmethylene-2-(2-nitro-phenylamino)-thiazol-4-one;
2-(2-Bromo-6-fluoro-phenylamino)-5-quinolin-6-ylmethylene-thiazol-4-one;
2-(2-Methoxy-6-methyl-phenylamino)-5-quinolin-6-ylmethylene-thiazol-4-one;
5-Quinolin-6-ylmethylene-2-(2,3,4-trifluoro-phenylamino)-thiazol-4-one;
2-(2,6-Dichloro-phenylamino)-5-(2-oxo-2H-chromen-6-ylmethylene)-thiazol-4-one;
2-(2-Bromo-phenylamino)-5-(5-pyridin-2-yl-thiophen-2-ylmethylene)-thiazol-4-one;
2-(2-Bromo-phenylamino)-5-(1-oxy-pyridin-4-ylmethylene)-thiazol-4-one;
2-(2-Bromo-phenylamino)-5-(3-p-tolyl-benzo[c]isoxazol-5-ylmethylene)-thiazol-4-one;
2-(2-Bromo-phenylamino)-5-(3,4-dihydro-2H-benzo[b][1-6]dioxepin-7-ylmethylene)-thiazol-4-one;
5-Benzo[1,2,5]oxadiazol-5-ylmethylene-2-(2-bromo-phenylamino)-thiazol-4-one;
2-(2,6-Dichloro-phenylamino)-5-(2-methoxy-pyridin-3-ylmethylene)-thiazol-4-one;
2-(2-Chloro-phenylamino)-5-(6-methoxy-pyridin-3-ylmethylene)-thiazol-4-one;
2-(2-Chloro-5-trifluoromethyl-phenylamino)-5-quinolin-6-ylmethylene-thiazol-4-one;
2-(2-Bromo-phenylamino)-5-(4-hydroxy-3-methoxy-benzylidene)-thiazol-4-one;
5-(2,3-Dihydro-benzofuran-5-ylmethylene)-2-(2-methoxy-phenylamino)-thiazol-4-one;
2-(2-Nitro-phenylamino)-5-quinolin-6-ylmethylene-thiazol-4-one;
2-(2-Bromo-phenylamino)-5-(3,4-diamino-benzylidene)-thiazol-4-one;
5-[2-(2-Chloro-phenylimino)-4-oxo-thiazolidin-5-ylidenemethyl]-1-methyl-1H-pyridin-2-one;
2-(2-Chloro-5-nitro-phenylamino)-5-quinolin-6-ylmethylene-thiazol-4-one;
2-(5-Amino-2-chloro-phenylamino)-5-quinolin-6-ylmethylene-thiazol-4-one;
N-[4-Chloro-3-(4-oxo-5-quinolin-6-ylmethylene-4,5-dihydro-thiazol-2-ylamino)-phenyl]-acetamidine hydrochloride;
4-{[4-oxo-5-(6-quinolinylmethylidene)-4,5-dihydro-1,3-thiazol-2-yl]amino}benzamide;
3-{[4-oxo-5-(6-quinolinylmethylidene)-4,5-dihydro-1,3-thiazol-2-yl]amino}benzenesulfonamide;
4-{[4-oxo-5-(6-quinolinylmethylidene)-4,5-dihydro-1,3-thiazol-2-yl]amino}-N-2-pyridinylbenzenesulfonamide;
2-({4-[(4-methyl-1-piperazinyl)methyl]phenyl}amino)-5-(6-quinolinylmethylidene)-1,3-thiazol-4(5H)-one;
2-({4-[(methylsulfonyl)methyl]phenyl}amino)-5-(6-quinolinylmethylidene)-1,3-thiazol-4(5H)-one;
2-({3-[(methylsulfonyl)methyl]phenyl}amino)-5-(6-quinolinylmethylidene)-1,3-thiazol-4(5H)-one;
2-{[4-(4-methyl-1-piperazinyl)phenyl]amino}-5-(6-quinolinylmethylidene)-1,3-thiazol-4(5H)-one;
2-{[2-(3-chlorophenyl)ethyl]amino}-5-(6-quinolinylmethylidene)-1,3-thiazol-4(5H)-one;
4-(2-{[4-oxo-5-(6-quinolinylmethylidene)-4,5-dihydro-1,3-thiazol-2-yl]amino}ethyl)benzenesulfonamide;
3-{[4-oxo-5-(6-quinolinylmethylidene)-4,5-dihydro-1,3-thiazol-2-yl]amino}benzamide;
2-[(2,6-Difluoro-phenylamino)-methylene]-5-quinolin-6-ylmethylene-thiazolidin-4-one;
2-[(2,6-Difluoro-phenylamino)-methylene]-5-quinolin-6-ylmethylene-thiazolidin-4-one;
[2,4-Dichloro-5-(4-oxo-5-quinolin-6-ylmethylene-thiazolidin-2-ylideneamino)-phenoxy]-acetic acid;
2-[2,4-Dichloro-5-(2-methoxy-ethoxy)-phenylimino]-5-quinolin-6-ylmethylene-thiazolidin-4-one;
4-Chloro-3-(4-oxo-5-quinolin-6-ylmethylene-thiazolidin-2-ylideneamino)-benzoic acid;
[2,4-Dichloro-5-(4-oxo-5-quinolin-6-ylmethylene-thiazolidin-2-ylideneamino)-phenoxy]-acetic acid;
   and/or a pharmaceutically acceptable salt, hydrate, solvate or pro-drug thereof.

The invention also relates to a pharmaceutical composition including a therapeutically effective amount of a compound of formula I or II, or a salt, solvate, or a physiologically functional derivative thereof and one or more of pharmaceutically acceptable carriers, diluents and excipients.

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

Compounds of Formula (I) are included in the pharmaceutical compositions of the invention.

By the term “aryl” as used herein, unless otherwise defined, is meant a cyclic or polycyclic aromatic ring containing from 1 to 14 carbon atoms and optionally containing from one to five heteroatoms, provided that when the number of carbon atoms is 1 the aromatic ring contains at least four heteroatoms, when the number of carbon atoms is 2 the aromatic ring contains at least three heteroatoms, when the number of carbons is 3 the aromatic ring contains at least two heteroatoms and when the number of carbon atoms is 4 the aromatic ring contains at least one heteroatom.

By the term “C₁-C₁₂aryl” as used herein, unless otherwise defined, is meant phenyl, naphthalene, 3,4-methylenedioxyphenyl, pyridine, biphenyl, quinoline, pyrimidine, quinazoline, thiophene, thiazole, furan, pyrrole, pyrazole, imidazole, indole, indene, pyrazine, 1,3-dihydro-2H-benzimidazol, benzimidazol, benzothiohpene, tetrahydrobenzothiohpene and tetrazole.

The term “substituted” as used herein, unless otherwise defined, is meant that the subject chemical moiety has one or more substituents selected from the group consisting of: aryl,

aryl substituted with one or more subsitituents selected from alkyl, hydroxy, alkoxy, oxo, C₁-C₁₂aryl optionally substituted with one or more substituents selected from hydroxy, alkoxy oxo, cyano, amino, alkylamino, dialkylamino, alkyl and alkoxy, trifluoromethyl, —SO₂NR²¹R²², N-acylamino, —CO₂R²⁰, and halogen, cycloalkyl substituted with one or more subsititents selected from alkyl, hydroxy, alkoxy, trifluoromethyl, —SO₂NR²¹R²², amino, —CO₂R²⁰, N-acylamino and halogen,
cycloalkyl containing from 1 to 4 heteroatoms substituted with one or more substituents selected from alkyl, hydroxy, alkoxy, —SO₂NR²¹R²², amino, —CO₂R²⁰, trifluoromethyl, N-acylamino and halogen,
alkoxy substituted with one or more substituents selected form alkyl, —CO₂H, hydroxyl, C₁-C₁₂aryl, alkoxy, amino and halogen,
cycloalkyl, cycloalkyl containing from 1 to 4 heteroatoms, C₁-C₄alkylcycloalkyl containing from 1 to 3 heteroatomsC₁-C₄alkyl, —C(O)NHS(O)₂R²⁰, —(CH₂)_{g}NR²³S(O)₂R²⁰, hydroxyalkyl, alkoxy, —(CH₂)_{g}NR²¹R²², —C(O)NR²¹R²², —S(O)₂NR²¹R²², —(CH₂)_{g}N(R²⁰)C(O)ₙR²⁰, —(CH₂)_{g}N═C(H)R²⁰, —C(O)R²⁰, acyloxy, —SC₁-C₆alkyl, alkyl, —OCF₃, amino, hydroxy, alkylamino, acetamide, aminoalkyl, aminoalkoxy, alkylaminoalkoxy, dialkylaminoalkoxy, alkoxyalkylamide, alkoxyC₁-C₁₂aryl, C₁-C₁₂aryl, C₁-C₁₂arylalkyl, dialkylamino, N-acylamino, aminoalkylN-acylamino, —(CH₂)_{g}C(O)OR²⁰, —(CH₂)_{g}S(O)ₙR²³, nitro, cyano, oxo, halogen, trifluoromethyloxy and trifluoromethyl;
where g is 0 to 6, n is 0 to 2, R²³ is hydrogen or alkyl, each R²⁰ is independently selected form hydrogen, alkyl, C₁-C₆alkyloxyC₁-C₆alkyl, C₁-C₄alkylC(O)OC₁-C₄alkyl, amino, alkylamino, dialkylamino, aminoC₁-C₆alkyl, alkylaminoc₁-C₆alkyl, dialkylaminoC₁-C₆alkyl, —C(O)OH, alkoxy, aryloxy, arylamino, diarylamino, arylalkylamino, aryl, aryl substituted with one or more substituents selected from oxo, hydroxyl and alkyl, arylC₁-C₄alkyl optionally substituted with one or more substituents selected from oxo, hydroxy, halogen, alkoxy and alkyl, —CH₂C(O)cycloalkyl containing from 1 to 4 heteroatoms, cycloalkylC₁-C₄alkyl, C₁-C₄alkyl substituted with cycloalkyl containing from 1 to 4 heteroatoms, cycloalkyl, cycloalkyl substituted with one or more substituents selected from oxo, hydroxyl and alkyl, cycloalkyl containing from 1 to 4 heteroatoms, cycloalkyl containing from 1 to 4 heteroatoms substituted with one or more substituents selected from oxo, hydroxyl and alkyl, and trifluoromethyl, and R²¹ and R²² are independently selected form hydrogen, alkyl, C₁-C₆alkyl substituted with one or more substituents selected from hydroxy, amino, ═NH, and ≡N, —S(O)₂aryl, —S(O)₂alkyl, C₁-C₁₂aryl, cycloalkyl containing from 1 to 4 heteroatoms, cycloalkyl containing from 1 to 4 heteroatoms substituted with one or more substituents selected from oxo, hydroxy, and alkyl, cycloalkyl, cycloalkyl substituted with one or more substituents selected from oxo, hydroxy, and alkyl, arylC₁-C₆alkyl optionally substituted with one or more substituents selected from oxo, hydroxy, and alkyl, cycloalkyl containing from 1 to 4 heteroatoms optionally substituted with one or more substituents selected from oxo, hydroxyl and alkyl, C₁-C₆alkoxy, C₁-C₄alkyloxyC₁-C₄alkyl, aryl and trifluoromethyl.

By the term “naphthyridin-6-yl” as used herein, is meant 1,5-naphthyridin-6-yl, 1,7-naphthyridin-6-yl, and 1,8-naphthyridin-6-yl.

By the term “alkoxy” as used herein is meant —Oalkyl where alkyl is as described herein including —OCH₃ and —OC(CH₃)₂CH₃.

The term “cycloalkyl” as used herein unless otherwise defined, is meant a nonaromatic, unsaturated or saturated, cyclic or polycyclic C₃-C₁₂.

Examples of cycloalkyl and substituted cycloalkyl substituents as used herein include: cyclohexyl, aminocyclohexyl, cyclobutyl, aminocyclobutyl, 4-hydroxy-cyclohexyl, 2-ethylcyclohexyl, propyl-4-methoxycyclohexyl, 4-methoxycyclohexyl, 4-carboxycyclohexyl, cyclopropyl, aminocyclopentyl, and cyclopentyl.

The term “cycloalkyl containing from 1 to 4 heteroatoms” and the term “cycloalkyl containing from 1 to 3 heteroatoms” as used herein unless otherwise defined, is meant a nonaromatic, unsaturated or saturated, cyclic or polycyclic ring containing from 1 to 12 carbons and containing from one to four heteroatoms or from one to three heteroatoms (respectively), provided that when the number of carbon atoms is 1 the aromatic ring contains at least four heteroatoms (applicable only where “cycloalkyl containing from 1 to 4 heteroatoms” is indicated), when the number of carbon atoms is 2 the aromatic ring contains at least three heteroatoms, when the number of carbon atoms is 3 the nonaromatic ring contains at least two heteroatoms and when the number of carbon atoms is 4 the nonaromatic ring contains at least one heteroatom.

Examples of cycloalkyl containing from 1 to 4 heteroatoms, cycloalkyl containing from 1 to 3 heteroatoms, substituted cycloalkyl containing from 1 to 4 heteroatoms and substituted cycloalkyl containing from 1 to 3 heteroatoms as used herein include: piperidine, piperazine, pyrrolidine, 3-methylaminopyrrolidine, piperazine, tetrazole, hexahydrodiazepine and morpholine.

By the term “acyloxy” as used herein is meant —OC(O)alkyl where alkyl is as described herein. Examples of acyloxy substituents as used herein include: —OC(O)CH₃, —OC(O)CH(CH₃)₂ and —OC(O)(CH₂)₃CH₃.

By the term “N-acylamino” as used herein is meant —N(H)C(O)alkyl, where alkyl is as described herein. Examples of N-acylamino substituents as used herein include: —N(H)C(O)CH₃, —N(H)C(O)CH(CH₃)₂ and —N(H)C(O)(CH₂)₃CH₃.

By the term “aryloxy” as used herein is meant —Oaryl where aryl is phenyl, naphthyl, 3,4-methylenedioxyphenyl, pyridyl or biphenyl optionally substituted with one or more substituents selected from the group consisting of: alkyl, hydroxyalkyl, alkoxy, trifluoromethyl, acyloxy, amino, N-acylamino, hydroxy, —(CH₂)_{g}C(O)OR²⁵, —S(O)ₙR²⁵, nitro, cyano, halogen and protected —OH, where g is 0-6, R²⁵ is hydrogen or alkyl, and n is 0-2. Examples of aryloxy substituents as used herein include: phenoxy, 4-fluorophenyloxy and biphenyloxy.

By the term “heteroatom” as used herein is meant oxygen, nitrogen or sulfur.

By the term “halogen” as used herein is meant a substituent selected from bromide, iodide, chloride and fluoride.

By the term “alkyl” and derivatives thereof and in all carbon chains as used herein, including alkyl chains defined by the term “—(CH₂)ₙ”, “—(CH₂)ₘ” and the like, is meant a linear or branched, saturated or unsaturated hydrocarbon chain, and unless otherwise defined, the carbon chain will contain from 1 to 12 carbon atoms.

Examples of alkyl and substituted alkyl substituents as used herein include:

—CH₃, —CH₂—CH₃, —CH₂—CH₂—CH₃, —CH(CH₃)₂, —CH₂—CH₂—C(CH₃)₃, —CH₂—CF₃, —C≡C—C(CH₃)₃, —C≡C—CH₂—OH, cyclopropylmethyl, —CH₂—C(CH₃)₂—CH₂—NH₂, C≡C—C₆H₅, —C≡C—C(CH₃)₂—OH, —CH₂—CH(OH)—CH(OH)—CH(OH)—CH(OH)—CH₂—OH, piperidinylmethyl, methoxyphenylethyl, —C(CH₃)₃, —(CH₂)₃—CH₃, —CH₂—CH(CH₃)₂, —CH(CH₃)—CH₂—CH₃, —CH═CH₂, and —C≡C—CH₃.

By the term “treating” and derivatives thereof as used herein, is meant prophylatic and therapeutic therapy.

As used herein, the term “optionally” means that the subsequently described event(s) may or may not occur, and includes both event(s), which occur, and events that do not occur.

As used herein, the crisscrossed double bond indicated by the symbol [symbol] denotes Z and/or E stereochemistry around the double bond. In other words a compound of formula I or II can be either in the Z or E stereochemistry around this double bond, or a compound of formula I or II can also be in a mixture of Z and E stereochemistry around the double bond. However, in formulas I and II, the preferred compounds have Z stereochemistry around the double bond to which radical Q is attached.

The compounds of Formulas I and II naturally may exist in one tautomeric form or in a mixture of tautomeric forms. For example, for sake simplicity, compounds of formula I and II are expressed in one tautomeric form, usually as an exo form, i.e.

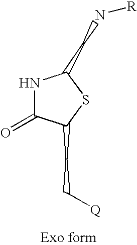

However, a person of ordinary skill can readily appreciate, the compounds of formulas I and II can also exist in endo forms.

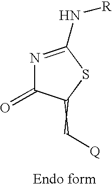

The present invention contemplates all possible tautomeric forms.

Certain compounds described herein may contain one or more chiral atoms, or may otherwise be capable of existing as two enantiomers, or two or more diastereoisomers. Accordingly, the compounds of this invention include mixtures of enantiomers/diastereoisomers as well as purified enantiomers/diastereoisomers or enantiomerically/diastereoisomerically enriched mixtures. Also included within the scope of the invention are the individual isomers of the compounds represented by formula I or II above as well as any wholly or partially equilibrated mixtures thereof. The present invention also covers the individual isomers of the compounds represented by the formulas above as mixtures with isomers thereof in which one or more chiral centers are inverted. Further, an example of a possible tautomer is an oxo substituent in place of a hydroxy substituent. Also, as stated above, it is understood that all tautomers and mixtures of tautomers are included within the scope of the compounds of Formula I or II.

Compounds of Formula (I) are included in the pharmaceutical compositions of the invention. Where a —COOH or —OH group is present, pharmaceutically acceptable esters can be employed, for example methyl, ethyl, pivaloyloxymethyl, and the like for —COOH, and acetate maleate and the like for —OH, and those esters known in the art for modifying solubility or hydrolysis characteristics, for use as sustained release or prodrug formulations.

It has now been found that compounds of the present invention are inhibitors of the Phosphatoinositides 3-kinases (PI3Ks). When the phosphatoinositides 3-kinase (PI3K) enzyme is inhibited by a compound of the present invention, PI3K is unable to exert its enzymatic, biological and/or pharmacological effects. The compounds of the present invention are therefore useful in the treatment of autoimmune disorders, inflammatory diseases, cardiovascular diseases, neurodegenerative diseases, allergy, asthma, pancreatitis, multiorgan failure, kidney diseases, platelet aggregation, cancer, sperm motility, transplantation rejection, graft rejection and lung injuries.

The compounds of Formula (I) are useful as medicaments in particular for the treatment of autoimmune disorders, inflammatory diseases, cardiovascular diseases, neurodegenerative diseases, allergy, asthma, pancreatitis, multiorgan failure, kidney diseases, platelet aggregation, cancer, sperm motility, transplantation rejection, graft rejection and lung injuries. According to one embodiment of the present invention, the compounds of Formula (I) are inhibitors of one or more phosphatoinositides 3-kinases (PI3Ks), suitably, Phosphatoinositides 3-kinase γ (PI3Kγ), Phosphatoinositides 3-kinase γ (PI3Kα), Phosphatoinositides 3-kinase γ (PI3Kβ), and/or Phosphatoinositides 3-kinase γ (PI3Kδ).

Compounds according to Formula (I) are suitable for the modulation, notably the inhibition of the activity of phosphatoinositides 3-kinases (PI3K), suitably phosphatoinositides 3-kinase (PI3Kγ). Therefore the compounds of the present invention are also useful for the treatment of disorders which are mediated by PI3Ks. Said treatment involves the modulation—notably the inhibition or the down regulation—of the phosphatoinositides 3-kinases.

Suitably, the compounds of the present invention are used for the preparation of a medicament for the treatment of a disorder selected from multiple sclerosis, psoriasis, rheumatoid arthritis, systemic lupus erythematosis, inflammatory bowel disease, lung inflammation, thrombosis or brain infection/inflammation, such as meningitis or encephalitis, Alzheimer's disease, Huntington's disease, CNS trauma, stroke or ischemic conditions, cardiovascular diseases such as athero-sclerosis, heart hypertrophy, cardiac myocyte dysfunction, elevated blood pressure or vasoconstriction.

Suitably, the compounds of Formula (I) are useful for the treatment of autoimmune diseases or inflammatory diseases such as multiple sclerosis, psoriasis, rheumatoid arthritis, systemic lupus erythematosis, inflammatory bowel disease, lung inflammation, thrombosis or brain infection/inflammation such as meningitis or encephalitis.

Suitably, the compounds of Formula (I) are useful for the treatment of neurodegenerative diseases including multiple sclerosis, Alzheimer's disease, Huntington's disease, CNS trauma, stroke or ischemic conditions.

Suitably, the compounds of Formula (I) are useful for the treatment of cardiovascular diseases such as atherosclerosis, heart hypertrophy, cardiac myocyte dysfunction, elevated blood pressure or vasoconstriction.

Suitably, the compounds of Formula (I) are useful for the treatment of chronic obstructive pulmonary disease, anaphylactic shock fibrosis, psoriasis, allergic diseases, asthma, stroke, ischemic conditions, ischemia-reperfusion, platelets aggregation/activation, skeletal muscle atrophy/hypertrophy, leukocyte recruitment in cancer tissue, angiogenesis, invasion metastasis, in particular melanoma, Karposi's sarcoma, acute and chronic bacterial and virual infections, sepsis, transplantation rejection, graft rejection, glomerulo sclerosis, glomerulo nephritis, progressive renal fibrosis, endothelial and epithelial injuries in the lung, and lung airway inflammation.

Because the pharmaceutically active compounds of the present invention are active as PI3 kinase inhibitors, particularly the compounds that inhibit PI3Kα, either selectively or in conjunction with one or more of PI3Kδ, PI3Kβ, and/or PI3Kγ, they exhibit therapeutic utility in treating cancer.

Suitably, the present invention relates to a method for treating or lessening the severity of a cancer selected from brain (gliomas), glioblastomas, Bannayan-Zonana syndrome, Cowden disease, Lhermitte-Duclos disease, breast, colon, head and neck, kidney, lung, liver, melanoma, ovarian, pancreatic, prostate, sarcoma and thyroid.

Suitably, the present invention relates to a method for treating or lessening the severity of a cancer selected from ovarian, pancreatic, breast, prostate and leukemia.

When a compound of Formula (I) is administered for the treatment of cancer, the term “co-administering” and derivatives thereof as used herein is meant either simultaneous administration or any manner of separate sequential administration of a PI3 kinase inhibiting compound, as described herein, and a further active ingredient or ingredients, known to be useful in the treatment of cancer, including chemotherapy and radiation treatment. The term further active ingredient or ingredients, as used herein, includes any compound or therapeutic agent known to or that demonstrates advantageous properties when administered to a patient in need of treatment for cancer. Preferably, if the administration is not simultaneous, the compounds are administered in a close time proximity to each other. Furthermore, it does not matter if the compounds are administered in the same dosage form, e.g. one compound may be administered topically and another compound may be administered orally.

Typically, any anti-neoplastic agent that has activity versus a susceptible tumor being treated may be co-administered in the treatment of cancer in the present invention. Examples of such agents can be found in Cancer Principles and Practice f Oncology by V. T. Devita and S. Hellman (editors), 6^{th} edition (Feb. 15, 2001), Lippincott Williams & Wilkins Publishers. A person of ordinary skill in the art would be able to discern which combinations of agents would be useful based on the particular characteristics of the drugs and the cancer involved. Typical anti-neoplastic agents useful in the present invention include, but are not limited to, anti-microtubule agents such as diterpenoids and vinca alkaloids; platinum coordination complexes; alkylating agents such as nitrogen mustards, oxazaphosphorines, alkylsulfonates, nitrosoureas, and triazenes; antibiotic agents such as anthracyclins, actinomycins and bleomycins; topoisomerase II inhibitors such as epipodophyllotoxins; antimetabolites such as purine and pyrimidine analogues and anti-folate compounds; topoisomerase I inhibitors such as camptothecins; hormones and hormonal analogues; signal transduction pathway inhibitors; non-receptor tyrosine kinase angiogenesis inhibitors; immunotherapeutic agents; proapoptotic agents; and cell cycle signaling inhibitors.

Examples of a further active ingredient or ingredients for use in combination or co-administered with the present PI3 kinase inhibiting compounds are chemotherapeutic agents.

Anti-microtubule or anti-mitotic agents are phase specific agents active against the microtubules of tumor cells during M or the mitosis phase of the cell cycle. Examples of anti-microtubule agents include, but are not limited to, diterpenoids and vinca alkaloids.

Diterpenoids, which are derived from natural sources, are phase specific anti-cancer agents that operate at the G₂/M phases of the cell cycle. It is believed that the diterpenoids stabilize the β-tubulin subunit of the microtubules, by binding with this protein. Disassembly of the protein appears then to be inhibited with mitosis being arrested and cell death following. Examples of diterpenoids include, but are not limited to, paclitaxel and its analog docetaxel.

Paclitaxel, 5β,20-epoxy-1,2α,4,7β,10β,13α-hexa-hydroxytax-11-en-9-one 4,10-diacetate 2-benzoate 13-ester with (2R,3S)—N-benzoyl-3-phenylisoserine; is a natural diterpene product isolated from the Pacific yew tree Taxus brevifolia and is commercially available as an injectable solution TAXOL®. It is a member of the taxane family of terpenes. It was first isolated in 1971 by Wani et al. J. Am. Chem., Soc., 93:2325. 1971), who characterized its structure by chemical and X-ray crystallographic methods. One mechanism for its activity relates to paclitaxel's capacity to bind tubulin, thereby inhibiting cancer cell growth. Schiff et al., Proc. Natl, Acad, Sci. USA, 77:1561-1565 (1980); Schiff et al., Nature, 277:665-667 (1979); Kumar, J. Biol, Chem, 256: 10435-10441 (1981). For a review of synthesis and anticancer activity of some paclitaxel derivatives see: D. G. I. Kingston et al., Studies in Organic Chemistry vol. 26, entitled “New trends in Natural Products Chemistry 1986”, Attaur-Rahman, P. W. Le Quesne, Eds. (Elsevier, Amsterdam, 1986) pp 219-235.

Paclitaxel has been approved for clinical use in the treatment of refractory ovarian cancer in the United States (Markman et al., Yale Journal of Biology and Medicine, 64:583, 1991; McGuire et al., Ann. Intem, Med., 111:273, 1989) and for the treatment of breast cancer (Holmes et al., J. Nat. Cancer Inst., 83:1797, 1991.) It is a potential candidate for treatment of neoplasms in the skin (Einzig et. al., Proc. Am. Soc. Clin. Oncol., 20:46) and head and neck carcinomas (Forastire et. al., Sem. Oncol., 20:56, 1990). The compound also shows potential for the treatment of polycystic kidney disease (Woo et. al., Nature, 368:750. 1994), lung cancer and malaria. Treatment of patients with paclitaxel results in bone marrow suppression (multiple cell lineages, Ignoff, R. J. et. al, Cancer Chemotherapy Pocket Guide, 1998) related to the duration of dosing above a threshold concentration (50 nM) (Kearns, C. M. et. al., Seminars in Oncology, 3(6) p. 16-23, 1995).

Docetaxel, (2R,3S)—N-carboxy-3-phenylisoserine,N-tert-butyl ester, 13-ester with 5β-20-epoxy-1,2α,4,7β,10β,13α-hexahydroxytax-11-en-9-one 4-acetate 2-benzoate, trihydrate; is commercially available as an injectable solution as TAXOTERE®. Docetaxel is indicated for the treatment of breast cancer. Docetaxel is a semisynthetic derivative of paclitaxel q.v., prepared using a natural precursor, 10-deacetyl-baccatin III, extracted from the needle of the European Yew tree. The dose limiting toxicity of docetaxel is neutropenia.

Vinca alkaloids are phase specific anti-neoplastic agents derived from the periwinkle plant. Vinca alkaloids act at the M phase (mitosis) of the cell cycle by binding specifically to tubulin. Consequently, the bound tubulin molecule is unable to polymerize into microtubules. Mitosis is believed to be arrested in metaphase with cell death following. Examples of vinca alkaloids include, but are not limited to, vinblastine, vincristine, and vinorelbine.

Vinblastine, vincaleukoblastine sulfate, is commercially available as VELBAN® as an injectable solution. Although, it has possible indication as a second line therapy of various solid tumors, it is primarily indicated in the treatment of testicular cancer and various lymphomas including Hodgkin's Disease; and lymphocytic and histiocytic lymphomas. Myelosuppression is the dose limiting side effect of vinblastine.

Vincristine, vincaleukoblastine, 22-oxo-, sulfate, is commercially available as ONCOVIN® as an injectable solution. Vincristine is indicated for the treatment of acute leukemias and has also found use in treatment regimens for Hodgkin's and non-Hodgkin's malignant lymphomas. Alopecia and neurologic effects are the most common side effect of vincristine and to a lesser extent myelosupression and gastrointestinal mucositis effects occur.

Vinorelbine, 3′,4′-didehydro-4′-deoxy-C′-norvincaleukoblastine [R—(R*,R*)-2,3-dihydroxybutanedioate (1:2)(salt)], commercially available as an injectable solution of vinorelbine tartrate (NAVELBINE®), is a semisynthetic vinca alkaloid. Vinorelbine is indicated as a single agent or in combination with other chemotherapeutic agents, such as cisplatin, in the treatment of various solid tumors, particularly non-small cell lung, advanced breast, and hormone refractory prostate cancers. Myelosuppression is the most common dose limiting side effect of vinorelbine.

Platinum coordination complexes are non-phase specific anti-cancer agents, which are interactive with DNA. The platinum complexes enter tumor cells, undergo, aquation and form intra- and interstrand crosslinks with DNA causing adverse biological effects to the tumor. Examples of platinum coordination complexes include, but are not limited to, cisplatin and carboplatin.

Cisplatin, cis-diamminedichloroplatinum, is commercially available as PLATINOL® as an injectable solution. Cisplatin is primarily indicated in the treatment of metastatic testicular and ovarian cancer and advanced bladder cancer. The primary dose limiting side effects of cisplatin are nephrotoxicity, which may be controlled by hydration and diuresis, and ototoxicity.

Carboplatin, platinum, diammine [1,1-cyclobutane-dicarboxylate(2-)-O,O′], is commercially available as PARAPLATIN® as an injectable solution. Carboplatin is primarily indicated in the first and second line treatment of advanced ovarian carcinoma. Bone marrow suppression is the dose limiting toxicity of carboplatin.

Alkylating agents are non-phase anti-cancer specific agents and strong electrophiles. Typically, alkylating agents form covalent linkages, by alkylation, to DNA through nucleophilic moieties of the DNA molecule such as phosphate, amino, sulfhydryl, hydroxyl, carboxyl, and imidazole groups. Such alkylation disrupts nucleic acid function leading to cell death. Examples of alkylating agents include, but are not limited to, nitrogen mustards such as cyclophosphamide, melphalan, and chlorambucil; alkyl sulfonates such as busulfan; nitrosoureas such as carmustine; and triazenes such as dacarbazine.

Cyclophosphamide, 2-[bis(2-chloroethyl)amino]tetrahydro-2H-1,3,2-oxazaphosphorine 2-oxide monohydrate, is commercially available as an injectable solution or tablets as CYTOXAN®. Cyclophosphamide is indicated as a single agent or in combination with other chemotherapeutic agents, in the treatment of malignant lymphomas, multiple myeloma, and leukemias. Alopecia, nausea, vomiting and leukopenia are the most common dose limiting side effects of cyclophosphamide.

Melphalan, 4-[bis(2-chloroethyl)amino]-L-phenylalanine, is commercially available as an injectable solution or tablets as ALKERAN®. Melphalan is indicated for the palliative treatment of multiple myeloma and non-resectable epithelial carcinoma of the ovary. Bone marrow suppression is the most common dose limiting side effect of melphalan.

Chlorambucil, 4-[bis(2-chloroethyl)amino]benzenebutanoic acid, is commercially available as LEUKERAN® tablets. Chlorambucil is indicated for the palliative treatment of chronic lymphatic leukemia, and malignant lymphomas such as lymphosarcoma, giant follicular lymphoma, and Hodgkin's disease. Bone marrow suppression is the most common dose limiting side effect of chlorambucil.

Busulfan, 1,4-butanediol dimethanesulfonate, is commercially available as MYLERAN® TABLETS. Busulfan is indicated for the palliative treatment of chronic myelogenous leukemia. Bone marrow suppression is the most common dose limiting side effects of busulfan.

Carmustine, 1,3-[bis(2-chloroethyl)-1-nitrosourea, is commercially available as single vials of lyophilized material as BiCNU®. Carmustine is indicated for the palliative treatment as a single agent or in combination with other agents for brain tumors, multiple myeloma, Hodgkin's disease, and non-Hodgkin's lymphomas. Delayed myelosuppression is the most common dose limiting side effects of carmustine.

Dacarbazine, 5-(3,3-dimethyl-1-triazeno)-imidazole-4-carboxamide, is commercially available as single vials of material as DTIC-Dome®. Dacarbazine is indicated for the treatment of metastatic malignant melanoma and in combination with other agents for the second line treatment of Hodgkin's Disease. Nausea, vomiting, and anorexia are the most common dose limiting side effects of dacarbazine.

Antibiotic anti-neoplastics are non-phase specific agents, which bind or intercalate with DNA. Typically, such action results in stable DNA complexes or strand breakage, which disrupts ordinary function of the nucleic acids leading to cell death. Examples of antibiotic anti-neoplastic agents include, but are not limited to, actinomycins such as dactinomycin, anthrocyclins such as daunorubicin and doxorubicin; and bleomycins.

Dactinomycin, also know as Actinomycin D, is commercially available in injectable form as COSMEGEN®. Dactinomycin is indicated for the treatment of Wilm's tumor and rhabdomyosarcoma. Nausea, vomiting, and anorexia are the most common dose limiting side effects of dactinomycin.

Daunorubicin, (8S-cis-)-8-acetyl-10-[(3-amino-2,3,6-trideoxy-α-L-lyxo-hexopyranosyl)oxy]-7,8,9,10-tetrahydro-6,8,11-trihydroxy-1-methoxy-5,12 naphthacenedione hydrochloride, is commercially available as a liposomal injectable form as DAUNOXOME® or as an injectable as CERUBIDINE®. Daunorubicin is indicated for remission induction in the treatment of acute nonlymphocytic leukemia and advanced HIV associated Kaposi's sarcoma. Myelosuppression is the most common dose limiting side effect of daunorubicin.

Doxorubicin, (8S,10S)-10-[(3-amino-2,3,6-trideoxy-α-L-lyxo-hexopyranosyl)oxy]-8-glycoloyl, 7,8,9,10-tetrahydro-6,8,11-trihydroxy-1-methoxy-5,12 naphthacenedione hydrochloride, is commercially available as an injectable form as RUBEX® or ADRIAMYCIN RDF®. Doxorubicin is primarily indicated for the treatment of acute lymphoblastic leukemia and acute myeloblastic leukemia, but is also a useful component in the treatment of some solid tumors and lymphomas. Myelosuppression is the most common dose limiting side effect of doxorubicin.

Bleomycin, a mixture of cytotoxic glycopeptide antibiotics isolated from a strain of Streptomyces verticillus, is commercially available as BLENOXANE®. Bleomycin is indicated as a palliative treatment, as a single agent or in combination with other agents, of squamous cell carcinoma, lymphomas, and testicular carcinomas. Pulmonary and cutaneous toxicities are the most common dose limiting side effects of bleomycin.

Topoisomerase II inhibitors include, but are not limited to, epipodophyllotoxins.

Epipodophyllotoxins are phase specific anti-neoplastic agents derived from the mandrake plant. Epipodophyllotoxins typically affect cells in the S and G₂ phases of the cell cycle by forming a ternary complex with topoisomerase II and DNA causing DNA strand breaks. The strand breaks accumulate and cell death follows. Examples of epipodophyllotoxins include, but are not limited to, etoposide and teniposide.

Etoposide, 4′-demethyl-epipodophyllotoxin 9[4,6-0-(R)-ethylidene-β-D-glucopyranoside], is commercially available as an injectable solution or capsules as VePESID® and is commonly known as VP-16. Etoposide is indicated as a single agent or in combination with other chemotherapy agents in the treatment of testicular and non-small cell lung cancers. Myelosuppression is the most common side effect of etoposide. The incidence of leucopenia tends to be more severe than thrombocytopenia.

Teniposide, 4′-demethyl-epipodophyllotoxin 9[4,6-0-(R)-thenylidene-β-D-glucopyranoside], is commercially available as an injectable solution as VUMON® and is commonly known as VM-26. Teniposide is indicated as a single agent or in combination with other chemotherapy agents in the treatment of acute leukemia in children. Myelosuppression is the most common dose limiting side effect of teniposide. Teniposide can induce both leucopenia and thrombocytopenia.

Antimetabolite neoplastic agents are phase specific anti-neoplastic agents that act at S phase (DNA synthesis) of the cell cycle by inhibiting DNA synthesis or by inhibiting purine or pyrimidine base synthesis and thereby limiting DNA synthesis. Consequently, S phase does not proceed and cell death follows. Examples of antimetabolite anti-neoplastic agents include, but are not limited to, fluorouracil, methotrexate, cytarabine, mercaptopurine, thioguanine, and gemcitabine.

5-fluorouracil, 5-fluoro-2,4-(1H,3H) pyrimidinedione, is commercially available as fluorouracil. Administration of 5-fluorouracil leads to inhibition of thymidylate synthesis and is also incorporated into both RNA and DNA. The result typically is cell death. 5-fluorouracil is indicated as a single agent or in combination with other chemotherapy agents in the treatment of carcinomas of the breast, colon, rectum, stomach and pancreas. Myelosuppression and mucositis are dose limiting side effects of 5-fluorouracil. Other fluoropyrimidine analogs include 5-fluoro deoxyuridine (floxuridine) and 5-fluorodeoxyuridine monophosphate.

Cytarabine, 4-amino-1-β-D-arabinofuranosyl-2(1H)-pyrimidinone, is commercially available as CYTOSAR-U® and is commonly known as Ara-C. It is believed that cytarabine exhibits cell phase specificity at S-phase by inhibiting DNA chain elongation by terminal incorporation of cytarabine into the growing DNA chain. Cytarabine is indicated as a single agent or in combination with other chemotherapy agents in the treatment of acute leukemia. Other cytidine analogs include 5-azacytidine and 2′,2′-difluorodeoxycytidine (gemcitabine). Cytarabine induces leucopenia, thrombocytopenia, and mucositis.

Mercaptopurine, 1,7-dihydro-6H-purine-6-thione monohydrate, is commercially available as PURINETHOL®. Mercaptopurine exhibits cell phase specificity at S-phase by inhibiting DNA synthesis by an as of yet unspecified mechanism. Mercaptopurine is indicated as a single agent or in combination with other chemotherapy agents in the treatment of acute leukemia. Myelosuppression and gastrointestinal mucositis are expected side effects of mercaptopurine at high doses. A useful mercaptopurine analog is azathioprine.

Thioguanine, 2-amino-1,7-dihydro-6H-purine-6-thione, is commercially available as TABLOID®. Thioguanine exhibits cell phase specificity at S-phase by inhibiting DNA synthesis by an as of yet unspecified mechanism. Thioguanine is indicated as a single agent or in combination with other chemotherapy agents in the treatment of acute leukemia. Myelosuppression, including leucopenia, thrombocytopenia, and anemia, is the most common dose limiting side effect of thioguanine administration. However, gastrointestinal side effects occur and can be dose limiting. Other purine analogs include pentostatin, erythrohydroxynonyladenine, fludarabine phosphate, and cladribine.

Gemcitabine, 2′-deoxy-2′,2′-difluorocytidine monohydrochloride (β-isomer), is commercially available as GEMZAR®. Gemcitabine exhibits cell phase specificity at S-phase and by blocking progression of cells through the G1/S boundary. Gemcitabine is indicated in combination with cisplatin in the treatment of locally advanced non-small cell lung cancer and alone in the treatment of locally advanced pancreatic cancer. Myelosuppression, including leucopenia, thrombocytopenia, and anemia, is the most common dose limiting side effect of gemcitabine administration.

Methotrexate, N-[4-[[(2,4-diamino-6-pteridinyl)methyl]methylamino]benzoyl]-L-glutamic acid, is commercially available as methotrexate sodium. Methotrexate exhibits cell phase effects specifically at S-phase by inhibiting DNA synthesis, repair and/or replication through the inhibition of dyhydrofolic acid reductase which is required for synthesis of purine nucleotides and thymidylate. Methotrexate is indicated as a single agent or in combination with other chemotherapy agents in the treatment of choriocarcinoma, meningeal leukemia, non-Hodgkin's lymphoma, and carcinomas of the breast, head, neck, ovary and bladder. Myelosuppression (leucopenia, thrombocytopenia, and anemia) and mucositis are expected side effect of methotrexate administration.

Camptothecins, including, camptothecin and camptothecin derivatives are available or under development as Topoisomerase I inhibitors. Camptothecins cytotoxic activity is believed to be related to its Topoisomerase I inhibitory activity. Examples of camptothecins include, but are not limited to irinotecan, topotecan, and the various optical forms of 7-(4-methylpiperazino-methylene)-10,11-ethylenedioxy-20-camptothecin described below.

Irinotecan HCl, (4S)-4,11-diethyl-4-hydroxy-9-[(4-piperidinopiperidino) carbonyloxy]-1H-pyrano[3′,4′,6,7]indolizino[1,2-b]quinoline-3,14(4H, 12H)-dione hydrochloride, is commercially available as the injectable solution CAMPTOSAR®.

Irinotecan is a derivative of camptothecin which binds, along with its active metabolite SN-38, to the topoisomerase I—DNA complex. It is believed that cytotoxicity occurs as a result of irreparable double strand breaks caused by interaction of the topoisomerase I: DNA: irintecan or SN-38 ternary complex with replication enzymes. Irinotecan is indicated for treatment of metastatic cancer of the colon or rectum. The dose limiting side effects of irinotecan HCl are myelosuppression, including neutropenia, and GI effects, including diarrhea.

Topotecan HCl, (S)-10-[(dimethylamino)methyl]-4-ethyl-4,9-dihydroxy-1H-pyrano[3′,4′,6,7]indolizino[1,2-b]quinoline-3,14-(4H, 12H)-dione monohydrochloride, is commercially available as the injectable solution HYCAMTIN®. Topotecan is a derivative of camptothecin which binds to the topoisomerase I—DNA complex and prevents religation of singles strand breaks caused by Topoisomerase I in response to torsional strain of the DNA molecule. Topotecan is indicated for second line treatment of metastatic carcinoma of the ovary and small cell lung cancer. The dose limiting side effect of topotecan HCl is myelosuppression, primarily neutropenia.

Also of interest, is the camptothecin derivative of formula A following, currently under development, including the racemic mixture (R,S) form as well as the R and S enantiomers:

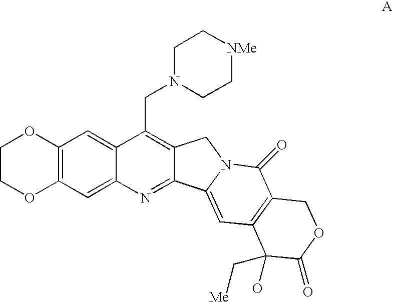

known by the chemical name “7-(4-methylpiperazino-methylene)-10,11-ethylenedioxy-20(R,S)-camptothecin (racemic mixture) or “7-(4-methylpiperazino-methylene)-10,11-ethylenedioxy-20(R)-camptothecin (R enantiomer) or “7-(4-methylpiperazino-methylene)-10,11-ethylenedioxy-20(S)-camptothecin (S enantiomer). Such compound as well as related compounds are described, including methods of making, in U.S. Pat. Nos. 6,063,923; 5,342,947; 5,559,235; 5,491,237 and pending U.S. patent application Ser. No. 08/977,217 filed Nov. 24, 1997.

Hormones and hormonal analogues are useful compounds for treating cancers in which there is a relationship between the hormone(s) and growth and/or lack of growth of the cancer. Examples of hormones and hormonal analogues useful in cancer treatment include, but are not limited to, adrenocorticosteroids such as prednisone and prednisolone which are useful in the treatment of malignant lymphoma and acute leukemia in children; aminoglutethimide and other aromatase inhibitors such as anastrozole, letrazole, vorazole, and exemestane useful in the treatment of adrenocortical carcinoma and hormone dependent breast carcinoma containing estrogen receptors; progestrins such as megestrol acetate useful in the treatment of hormone dependent breast cancer and endometrial carcinoma; estrogens, androgens, and anti-androgens such as flutamide, nilutamide, bicalutamide, cyproterone acetate and 5α-reductases such as finasteride and dutasteride, useful in the treatment of prostatic carcinoma and benign prostatic hypertrophy; anti-estrogens such as tamoxifen, toremifene, raloxifene, droloxifene, iodoxyfene, as well as selective estrogen receptor modulators (SERMS) such those described in U.S. Pat. Nos. 5,681,835, 5,877,219, and 6,207,716, useful in the treatment of hormone dependent breast carcinoma and other susceptible cancers; and gonadotropin-releasing hormone (GnRH) and analogues thereof which stimulate the release of leutinizing hormone (LH) and/or follicle stimulating hormone (FSH) for the treatment prostatic carcinoma, for instance, LHRH agonists and antagagonists such as goserelin acetate and luprolide.

Signal transduction pathway inhibitors are those inhibitors, which block or inhibit a chemical process which evokes an intracellular change. As used herein this change is cell proliferation or differentiation. Signal transduction inhibitors useful in the present invention include inhibitors of receptor tyrosine kinases, non-receptor tyrosine kinases, SH2/SH3domain blockers, serine/threonine kinases, phosphotidyl inositol-3 kinases, myo-inositol signaling, and Ras oncogenes.

Several protein tyrosine kinases catalyse the phosphorylation of specific tyrosyl residues in various proteins involved in the regulation of cell growth. Such protein tyrosine kinases can be broadly classified as receptor or non-receptor kinases.

Receptor tyrosine kinases are transmembrane proteins having an extracellular ligand binding domain, a transmembrane domain, and a tyrosine kinase domain. Receptor tyrosine kinases are involved in the regulation of cell growth and are generally termed growth factor receptors. Inappropriate or uncontrolled activation of many of these kinases, i.e. aberrant kinase growth factor receptor activity, for example by over-expression or mutation, has been shown to result in uncontrolled cell growth. Accordingly, the aberrant activity of such kinases has been linked to malignant tissue growth. Consequently, inhibitors of such kinases could provide cancer treatment methods. Growth factor receptors include, for example, epidermal growth factor receptor (EGFr), platelet derived growth factor receptor (PDGFr), erbB2, erbB4, vascular endothelial growth factor receptor (VEGFr), tyrosine kinase with immunoglobulin-like and epidermal growth factor homology domains (TIE-2), insulin growth factor-I (IGFI) receptor, macrophage colony stimulating factor (cfms), BTK, ckit, cmet, fibroblast growth factor (FGF) receptors, Trk receptors (TrkA, TrkB, and TrkC), ephrin (eph) receptors, and the RET protooncogene. Several inhibitors of growth receptors are under development and include ligand antagonists, antibodies, tyrosine kinase inhibitors and anti-sense oligonucleotides. Growth factor receptors and agents that inhibit growth factor receptor function are described, for instance, in Kath, John C., Exp. Opin. Ther. Patents (2000) 10(6):803-818; Shawver et al DDT Vol 2, No. 2 Feb. 1997; and Lofts, F. J. et al, “Growth factor receptors as targets”, New Molecular Targets for Cancer Chemotherapy, ed. Workman, Paul and Kerr, David, CRC press 1994, London.

Tyrosine kinases, which are not growth factor receptor kinases are termed non-receptor tyrosine kinases. Non-receptor tyrosine kinases useful in the present invention, which are targets or potential targets of anti-cancer drugs, include cSrc, Lck, Fyn, Yes, Jak, cAbl, FAK (Focal adhesion kinase), Brutons tyrosine kinase, and Bcr-Abl. Such non-receptor kinases and agents which inhibit non-receptor tyrosine kinase function are described in Sinh, S, and Corey, S. J., (1999) Journal of Hematotherapy and Stem Cell Research 8 (5): 465-80; and Bolen, J. B., Brugge, J. S., (1997) Annual review of Immunology. 15: 371-404.

SH2/SH3 domain blockers are agents that disrupt SH2 or SH3 domain binding in a variety of enzymes or adaptor proteins including, PI3-K p85 subunit, Src family kinases, adaptor molecules (Shc, Crk, Nck, Grb2) and Ras-GAP. SH2/SH3 domains as targets for anti-cancer drugs are discussed in Smithgall, T. E. (1995), Journal of Pharmacological and Toxicological Methods. 34(3) 125-32.

Inhibitors of Serine/Threonine Kinases including MAP kinase cascade blockers which include blockers of Raf kinases (rafk), Mitogen or Extracellular Regulated Kinase (MEKs), and Extracellular Regulated Kinases (ERKs); and Protein kinase C family member blockers including blockers of PKCs (alpha, beta, gamma, epsilon, mu, lambda, iota, zeta). IkB kinase family (IKKa, IKKb), PKB family kinases, AKT kinase family members, and TGF beta receptor kinases. Such Serine/Threonine kinases and inhibitors thereof are described in Yamamoto, T., Taya, S., Kaibuchi, K., (1999), Journal of Biochemistry. 126 (5) 799-803; Brodt, P, Samani, A., and Navab, R. (2000), Biochemical Pharmacology, 60.1101-1107; Massague, J., Weis-Garcia, F. (1996) Cancer Surveys. 27:41-64; Philip, P. A., and Harris, A. L. (1995), Cancer Treatment and Research. 78: 3-27, Lackey, K. et al Bioorganic and Medicinal Chemistry Letters, (10), 2000, 223-226; U.S. Pat. No. 6,268,391; and Martinez-lacaci, L., et al, Int. J. Cancer (2000), 88(1), 44-52.

Inhibitors of Phosphotidyl inositol-3 Kinase family members including blockers of PI3-kinase, ATM, DNA-PK, and Ku are also useful in the present invention. Such kinases are discussed in Abraham, R. T. (1996), Current Opinion in Immunology. 8 (3) 412-8; Canman, C. E., Lim, D. S. (1998), Oncogene 17 (25) 3301-3308; Jackson, S. P. (1997), International Journal of Biochemistry and Cell Biology. 29 (7):935-8; and Zhong, H. et al, Cancer res, (2000) 60(6), 1541-1545.

Also useful in the present invention are Myo-inositol signaling inhibitors such as phospholipase C blockers and Myoinositol analogues. Such signal inhibitors are described in Powis, G., and Kozikowski A., (1994) New Molecular Targets for Cancer Chemotherapy ed., Paul Workman and David Kerr, CRC press 1994, London.

Another group of signal transduction pathway inhibitors are inhibitors of Ras Oncogene. Such inhibitors include inhibitors of farnesyltransferase, geranyl-geranyl transferase, and CAAX proteases as well as anti-sense oligonucleotides, ribozymes and immunotherapy. Such inhibitors have been shown to block ras activation in cells containing wild type mutant ras, thereby acting as antiproliferation agents. Ras oncogene inhibition is discussed in Scharovsky, O. G., Rozados, V. R., Gervasoni, S. I. Matar, P. (2000), Journal of Biomedical Science. 7(4) 292-8; Ashby, M. N. (1998), Current Opinion in Lipidology. 9 (2) 99-102; and BioChim. Biophys. Acta, (19899) 1423(3):19-30.

As mentioned above, antibody antagonists to receptor kinase ligand binding may also serve as signal transduction inhibitors. This group of signal transduction pathway inhibitors includes the use of humanized antibodies to the extracellular ligand binding domain of receptor tyrosine kinases. For example Imclone C225 EGFR specific antibody (see Green, M. C. et al, Monoclonal Antibody Therapy for Solid Tumors, Cancer Treat. Rev., (2000), 26(4), 269-286); Herceptin® erbB2 antibody (see Tyrosine Kinase Signalling in Breast cancer:erbB Family Receptor Tyrosine Kinases, Breast cancer Res., 2000, 2(3), 176-183); and 2CB VEGFR2 specific antibody (see Brekken, R. A. et al, Selective Inhibition of VEGFR2Activity by a monoclonal Anti-VEGF antibody blocks tumor growth in mice, Cancer Res. (2000) 60, 5117-5124).

Non-receptor kinase angiogenesis inhibitors may also find use in the present invention. Inhibitors of angiogenesis related VEGFR and TIE2 are discussed above in regard to signal transduction inhibitors (both receptors are receptor tyrosine kinases). Angiogenesis in general is linked to erbB2/EGFR signaling since inhibitors of erbB2 and EGFR have been shown to inhibit angiogenesis, primarily VEGF expression. Thus, the combination of an erbB2/EGFR inhibitor with an inhibitor of angiogenesis makes sense. Accordingly, non-receptor tyrosine kinase inhibitors may be used in combination with the EGFR/erbB2 inhibitors of the present invention. For example, anti-VEGF antibodies, which do not recognize VEGFR (the receptor tyrosine kinase), but bind to the ligand; small molecule inhibitors of integrin (alphaᵥ beta₃) that will inhibit angiogenesis; endostatin and angiostatin (non-RTK) may also prove useful in combination with the disclosed erb family inhibitors. (See Bruns C J et al (2000), Cancer Res., 60: 2926-2935; Schreiber A B, Winkler M E, and Derynck R. (1986), Science, 232: 1250-1253; Yen L et al. (2000), Oncogene 19: 3460-3469).

Agents used in immunotherapeutic regimens may also be useful in combination with the compounds of formula (I). There are a number of immunologic strategies to generate an immune response against erbB2 or EGFR. These strategies are generally in the realm of tumor vaccinations. The efficacy of immunologic approaches may be greatly enhanced through combined inhibition of erbB2/EGFR signaling pathways using a small molecule inhibitor. Discussion of the immunologic/tumor vaccine approach against erbB2/EGFR are found in Reilly R T et al. (2000), Cancer Res. 60: 3569-3576; and Chen Y, Hu D, Eling D J, Robbins J, and Kipps T J. (1998), Cancer Res. 58: 1965-1971.

Agents used in proapoptotic regimens (e.g., bcl-2 antisense oligonucleotides) may also be used in the combination of the present invention. Members of the Bcl-2 family of proteins block apoptosis. Upregulation of bcl-2 has therefore been linked to chemoresistance. Studies have shown that the epidermal growth factor (EGF) stimulates anti-apoptotic members of the bcl-2 family (i.e., mcl-1). Therefore, strategies designed to downregulate the expression of bcl-2 in tumors have demonstrated clinical benefit and are now in Phase II/III trials, namely Genta's G3139 bcl-2 antisense oligonucleotide. Such proapoptotic strategies using the antisense oligonucleotide strategy for bcl-2 are discussed in Water J S et al. (2000), J. Clin. Oncol. 18: 1812-1823; and Kitada S et al. (1994), Antisense Res. Dev. 4: 71-79.

Cell cycle signalling inhibitors inhibit molecules involved in the control of the cell cycle. A family of protein kinases called cyclin dependent kinases (CDKs) and their interaction with a family of proteins termed cyclins controls progression through the eukaryotic cell cycle. The coordinate activation and inactivation of different cyclin/CDK complexes is necessary for normal progression through the cell cycle. Several inhibitors of cell cycle signalling are under development. For instance, examples of cyclin dependent kinases, including CDK2, CDK4, and CDK6 and inhibitors for the same are described in, for instance, Rosania et al, Exp. Opin. Ther. Patents (2000) 10(2):215-230.

In one embodiment, the cancer treatment method of the claimed invention includes the co-administration a compound of formula I and/or a pharmaceutically acceptable salt, hydrate, solvate or pro-drug thereof and at least one anti-neoplastic agent, such as one selected from the group consisting of anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine kinase angiogenesis inhibitors, immunotherapeutic agents, proapoptotic agents, and cell cycle signaling inhibitors.

Because the pharmaceutically active compounds of the present invention are active as PI3 kinase inhibitors, particularly the compounds that modulate/inhibit PI3Kγ, either selectively or in conjunction with one or more of PI3Kδ, PI3Kβ, and/or PI3Kα, they exhibit therapeutic utility in treating a disease state selected from: autoimmune disorders, inflammatory diseases, cardiovascular diseases, neurodegenerative diseases, allergy, asthma, pancreatitis, multiorgan failure, kidney diseases, platelet aggregation, sperm motility, transplantation rejection, graft rejection and lung injuries.

When a compound of Formula (I) is administered for the treatment of a disease state selected from: autoimmune disorders, inflammatory diseases, cardiovascular diseases, neurodegenerative diseases, allergy, asthma, pancreatitis, multiorgan failure, kidney diseases, platelet aggregation, sperm motility, transplantation rejection, graft rejection or lung injuries, the term “co-administering” and derivatives thereof as used herein is meant either simultaneous administration or any manner of separate sequential administration of a PI3 kinase inhibiting compound, as described herein, and a further active ingredient or ingredients, known to be useful in the treatment of autoimmune disorders, inflammatory diseases, cardiovascular diseases, neurodegenerative diseases, allergy, asthma, pancreatitis, multiorgan failure, kidney diseases, platelet aggregation, sperm motility, transplantation rejection, graft rejection and/or lung injuries.

The pharmaceutically active compounds within the scope of this invention are useful as PI3 Kinase inhibitors in mammals, particularly humans, in need thereof.

The present invention therefore provides a method of treating diseases associated with PI3 kinase inhibition, particularly: autoimmune disorders, inflammatory diseases, cardiovascular diseases, neurodegenerative diseases, allergy, asthma, pancreatitis, multiorgan failure, kidney diseases, platelet aggregation, cancer, sperm motility, transplantation rejection, graft rejection and lung injuries and other conditions requiring PI3 kinase modulation/inhibition, which comprises administering an effective compound of Formula (I) or a pharmaceutically acceptable salt, hydrate, solvate or pro-drug thereof. The compounds of Formula (I) also provide for a method of treating the above indicated disease states because of their ability to act as PI3 inhibitors. The drug may be administered to a patient in need thereof by any conventional route of administration, including, but not limited to, intravenous, intramuscular, oral, subcutaneous, intradermal, and parenteral.

The pharmaceutically active compounds of the present invention are incorporated into convenient dosage forms such as capsules, tablets, or injectable preparations. Solid or liquid pharmaceutical carriers are employed. Solid carriers include, starch, lactose, calcium sulfate dihydrate, terra alba, sucrose, talc, gelatin, agar, pectin, acacia, magnesium stearate, and stearic acid. Liquid carriers include syrup, peanut oil, olive oil, saline, and water. Similarly, the carrier or diluent may include any prolonged release material, such as glyceryl monostearate or glyceryl distearate, alone or with a wax. The amount of solid carrier varies widely but, preferably, will be from about 25 mg to about 1 g per dosage unit. When a liquid carrier is used, the preparation will be in the form of a syrup, elixir, emulsion, soft gelatin capsule, sterile injectable liquid such as an ampoule, or an aqueous or nonaqueous liquid suspension.

The pharmaceutical preparations are made following conventional techniques of a pharmaceutical chemist involving mixing, granulating, and compressing, when necessary, for tablet forms, or mixing, filling and dissolving the ingredients, as appropriate, to give the desired oral or parenteral products.

Doses of the presently invented pharmaceutically active compounds in a pharmaceutical dosage unit as described above will be an efficacious, nontoxic quantity preferably selected from the range of 0.001-100 mg/kg of active compound, preferably 0.001-50 mg/kg. When treating a human patient in need of a PI3K inhibitor, the selected dose is administered preferably from 1-6 times daily, orally or parenterally. Preferred forms of parenteral administration include topically, rectally, transdermally, by injection and continuously by infusion. Oral dosage units for human administration preferably contain from 0.05 to 3500 mg of active compound. Oral administration, which uses lower dosages is preferred. Parenteral administration, at high dosages, however, also can be used when safe and convenient for the patient. The above dosages relate to suitable amount of compound expressed as the free acid.

Optimal dosages to be administered may be readily determined by those skilled in the art, and will vary with the particular PI3 kinase inhibitor in use, the strength of the preparation, the mode of administration, and the advancement of the disease condition. Additional factors depending on the particular patient being treated will result in a need to adjust dosages, including patient age, weight, diet, and time of administration.

The method of this invention of inducing PI3 kinase inhibitory activity in mammals, including humans, comprises administering to a subject in need of such activity an effective PI3 kinase modulating/inhibiting amount of a pharmaceutically active compound of the present invention.

The invention also provides for the use of a compound of Formula (I) in the manufacture of a medicament for use as a PI3 kinase inhibitor.

The invention also provides for the use of a compound of Formula (I) in the manufacture of a medicament for use in therapy.

The invention also provides for the use of a compound of Formula (I) in the manufacture of a medicament for use in treating autoimmune disorders, inflammatory diseases, cardiovascular diseases, neurodegenerative diseases, allergy, asthma, pancreatitis, multiorgan failure, kidney diseases, platelet aggregation, cancer, sperm motility, transplantation rejection, graft rejection and lung injuries.

The invention also provides for a pharmaceutical composition for use as a PI3 inhibitor which comprises a compound of Formula (I) and a pharmaceutically acceptable carrier.

The invention also provides for a pharmaceutical composition for use in the treatment of autoimmune disorders, inflammatory diseases, cardiovascular diseases, neurodegenerative diseases, allergy, asthma, pancreatitis, multiorgan failure, kidney diseases, platelet aggregation, cancer, sperm motility, transplantation rejection, graft rejection and lung injuries, which comprises a compound of Formula (I) and a pharmaceutically acceptable carrier.

No unacceptable toxicological effects are expected when compounds of the invention are administered in accordance with the present invention.

In addition, the pharmaceutically active compounds of the present invention can be co-administered with further active ingredients, including compounds known to have utility when used in combination with a PI3 kinase inhibitor.

Without further elaboration, it is believed that one skilled in the art can, using the preceding description, utilize the present invention to its fullest extent. The following examples are, therefore, to be construed as merely illustrative and not a limitation of the scope of the present invention in any way.

For ease of illustration, the regiochemistry around the double bonds in the chemical formulas in the Examples are drawn as fixed for ease of representation; however, a skilled in the art will readily appreciate that the compounds will naturally assume more thermodynamically stable structure around the C═N (the imine) double bond if it exits as exo form. Further compounds can also exit in endo form. As stated before, the invention contemplates both endo and exo forms as well as both regioisomers around the exo imine bond. Further it is intended that both E and Z isomers are encompassed around the C═C double bond.

Compounds of general formula I may be prepared by methods known in the art of organic synthesis as set forth in part by the following synthesis schemes. In all of the schemes described below, it is well understood that protecting groups for sensitive or reactive groups are employed where necessary in accordance with general principles of chemistry. Protecting groups are manipulated according to standard methods of organic synthesis (T. W. Green and P. G. M. Wuts (1991) Protecting Groups in Organic Synthesis, John Wiley & Sons). These groups are removed at a convenient stage of the compound synthesis using methods that are readily apparent to those skilled in the art. The selection of processes as well as the reaction conditions and order of their execution shall be consistent with the preparation of compounds of formula I. Those skilled in the art will recognize if a stereocenter exists in compounds of formula I. Accordingly, the present invention includes both possible stereoisomers and includes not only racemic compounds but the individual enantiomers as well. When a compound is desired as a single enantiomer, it may be obtained by stereospecific synthesis or by resolution of the final product or any convenient intermediate. Resolution of the final product, an intermediate, or a starting material may be effected by any suitable method known in the art. See, for example, Stereochemistry of Organic Compounds by E. L. Eliel, S. H. Wilen, and L. N. Mander (Wiley-Interscience, 1994).

More particularly, the compounds of the formula I can be made by the process of either Scheme A or B or a variant thereof. Any person skilled in the art can readily adapt the process of either A or B, such the stoichemistry of the reagents, temperature, solvents, etc. to optimize the yield of the products desired.

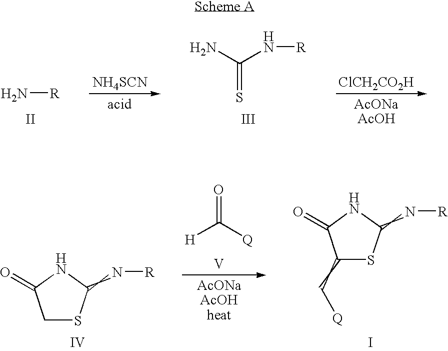

Briefly in Scheme A, a mixture of aniline derivative of formula II (1 equivalent) and NH4SCN (about 1.3 equivalent) in an acid (typically 4N—HCl) is heated to reflux at about 110 C° for 6 hours. After cooling, the mixture is treated with H₂O, which process usually forms a solid, followed by desiccation in vacuo to give a compound of formula III.

A mixture of formula III compound, ClCH₂CO₂H (1 equivalent), and AcONa (1 equivalent) in AcOH is heated to reflux at around 110 C° for about 4 h. The mixture is poured onto water thereby a solid is typically formed, which is isolated by filtration. The solid is washed with a solvent such as MeOH to afford a compound of formula IV.

A mixture of formula IV compound, an aldehyde of formula V (1 equivalent), AcONa (3 equivalent) in AcOH is heated to reflux at about 110 C° for about 10 to 48 hours. After cooling, a small portion of water was added until the solid forms. The solid is filtered and washed with a solvent such as MeOH, followed by desiccation in vacuo to afford a target product of formula I.

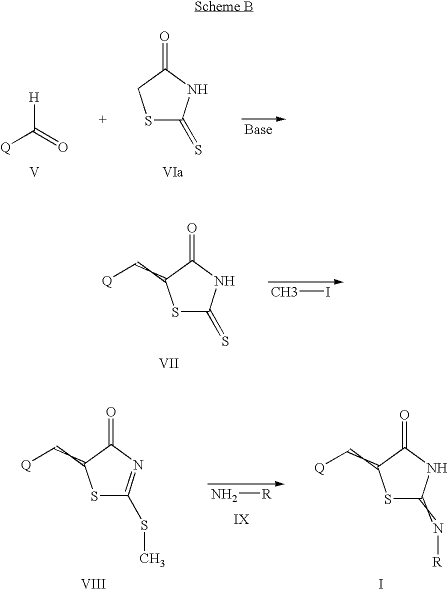

Briefly in Scheme B, a mixture of an aldehyde of formula V (1 equivalent), Rhodanine

(1 equivalent), sodium acetate (about 3 equivalents), and acetic acid was heated at around 110 C° for about 48 h. The reaction mixture is cooled to room temperature to afford a product of formula VII.

Then, to a room temperature suspension of VII (1 equivalent) in a suitable solvent such as ethanol was added Hunig's base (about 2 equivalents) followed by iodomethane (about 5 equivalents). Stirring the resultant suspension at room temperature for 3.5 h will yield a compound of VIII.

To a mixture of VIII (1 equivalent) and MS4A powder was added an amine of formula IX (1˜2 equivalent) and ethanol (dehydrated). The mixture was heated by microwave (SmithSynthesizer-Personal Chemistry) at about 110 C° for about 1200 seconds. Usually, the desired product of formula I can be obtained in about 20˜90% yield after purification.

In Schemes A and B, the meaning of R and Q are as defined in formula I.

All the starting materials are either known, commercially available or can be readily made by a routine method. For example, an aldehyde of formula V in which the radical Q is of the formula

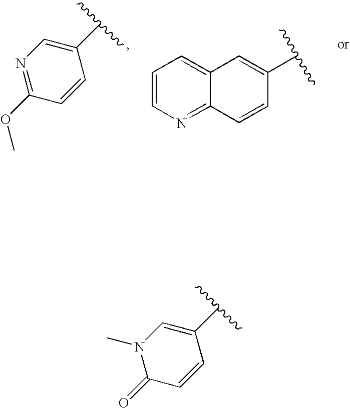

can be readily made by the following standard reaction steps.

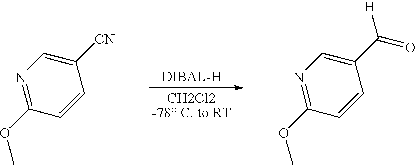

See Eur. J. Org. Chem., 1999, 2609˜2621.

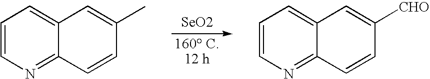

See J. Med. Chem., 2000, 43, 3878˜3894.

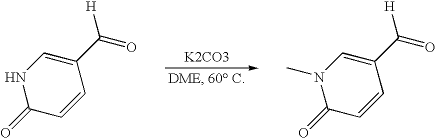

See J. Am. Chem. Soc., 1999, 121-6722˜4723.

In other embodiments, additional compounds of the invention can also be synthesized whereby a compound of formula I are first made by a process of Scheme A or B (or a variant thereof), and Q and R radicals in compounds of formula I thus made are further converted by routine organic reaction techniques into different Q and R groups. For such alterantives, see Schemes C, D and E.

### Biological Assays

The compounds of the present invention are tested to determine their inhibitory activity at PI3Kα, PI3Kδ, PI3Kβ and PI3Kγ according to the following.

For all PI3K isoforms:
1. Cloning, expression, purification, and characterization of the human Class Ia phosphoinositide 3-kinase isoforms: Meier, T. I.; Cook, J. A.; Thomas, J. E.; Radding, J. A.; Horn, C.; Lingaraj, T.; Smith, M. C. Protein Expr. Purif., 2004, 35(2), 218.
2. Competitive fluorescence polarization assays for the detection of phosphoinositide kinase and phosphatase activity: Drees, B. E.; Weipert, A.; Hudson, H.; Ferguson, C. G.; Chakravarty, L.; Prestwich, G. D. Comb. Chem. High Throughput. Screen., 2003, 6(4), 321.

### For PI3Kγ: WO 2005/011686 A1

### EXPERIMENTAL DETAILS

As used herein the symbols and conventions used in these processes, schemes and examples are consistent with those used in the contemporary scientific literature, for example, the Journal of the American Chemical Society or the Journal of Biological Chemistry. Standard single-letter or three-letter abbreviations are generally used to designate amino acid residues, which are assumed to be in the L-configuration unless otherwise noted. Unless otherwise noted, all starting materials were obtained from commercial suppliers and used without further purification. Specifically, the following abbreviations may be used in the examples and throughout the specification:
g (grams); mg (milligrams);
L (liters); mL (milliliters);
μL (microliters); psi (pounds per square inch);
M (molar); mM (millimolar);
i. v. (intravenous); Hz (Hertz);
MHz (megahertz); mol (moles);
mmol (millimoles); rt (room temperature);
min (minutes); h (hours);
mp (melting point); TLC (thin layer chromatography);
Tr (retention time); RP (reverse phase);
MeOH (methanol); i-PrOH (isopropanol);
TEA (triethylamine); TFA (trifluoroacetic acid);
TFAA (trifluoroacetic anhydride); THF (tetrahydrofuran);
DMSO (dimethylsulfoxide); AcOEt (ethyl acetate);
DME (1,2-dimethoxyethane); DCM (dichloromethane);
DCE (dichloroethane); DMF (N,N-dimethylformamide);
DMPU (N,N′-dimethylpropyleneurea); (CDI (1,1-carbonyldiimidazole);
IBCF (isobutyl chloroformate); HOAc (acetic acid);
HOSu (N-hydroxysuccinimide); HOBT (1-hydroxybenzotriazole);
mCPBA (meta-chloroperbenzoic acid; EDC (ethylcarbodiimide hydrochloride); BOC (tert-butyloxycarbonyl); FMOC (9-fluorenylmethoxycarbonyl); DCC (dicyclohexylcarbodiimide); CBZ (benzyloxycarbonyl);
Ac (acetyl); atm (atmosphere);
TMSE (2-(trimethylsilyl)ethyl); TMS (trimethylsilyl);
TIPS (triisopropylsilyl); TBS (t-butyldimethylsilyl);
DMAP (4-dimethylaminopyridine); BSA (bovine serum albumin)
ATP (adenosine triphosphate); HRP (horseradish peroxidase);
DMEM (Dulbecco's modified Eagle medium);
HPLC (high pressure liquid chromatography);
BOP (bis(2-oxo-3-oxazolidinyl)phosphinic chloride);
TBAF (tetra-n-butylammonium fluoride);
HBTU (O-Benzotriazole-1-yl-N,N,N′,N′-tetramethyluronium hexafluorophosphate).
HEPES (4-(2-hydroxyethyl)-1-piperazine ethane sulfonic acid);
DPPA (diphenylphosphoryl azide);
fHNO3 (fumed HNO3); and
EDTA (ethylenediaminetetraacetic acid).

All references to ether are to diethyl ether; brine refers to a saturated aqueous solution of NaCl. Unless otherwise indicated, all temperatures are expressed in ° C. (degrees Centigrade). All reactions are conducted under an inert atmosphere at room temperature unless otherwise noted.

¹H NMR spectra were recorded on a Varian VXR-300, a Varian Unity-300, a Varian Unity-400 instrument, a BruckerAVANCE-400, or a General Electric QE-300. Chemical shifts are expressed in parts per million (ppm, δ units). Coupling constants are in units of hertz (Hz). Splitting patterns describe apparent multiplicities and are designated as

s (singlet), d (doublet), t (triplet), q (quartet), quint (quintet), m (multiplet), br (broad).

Low-resolution mass spectra (MS) were recorded on a JOEL JMS-AX505HA, JOEL SX-102, or a SCIEX-APIiii spectrometer; LC-MS were recorded on a micromass 2MD and Waters 2690; high resolution MS were obtained using a JOEL SX-102A spectrometer. All mass spectra were taken under electrospray ionization (ESI), chemical ionization (CI), electron impact (EI) or by fast atom bombardment (FAB) methods. Infrared (IR) spectra were obtained on a Nicolet 510 FT-IR spectrometer using a 1-mm NaCl cell. Most of the reactions were monitored by thin-layer chromatography on 0.25 mm E. Merck silica gel plates (60F-254), visualized with UV light, 5% ethanolic phosphomolybdic acid or p-anisaldehyde solution. Flash column chromatography was performed on silica gel (230-400 mesh, Merck).

### Example 1

### 2-(2-Chloro-5-fluoro-phenylimino)-5-(2,3-dihydro-benzofuran-5-ylmethylene)-thiazolidin-4-one

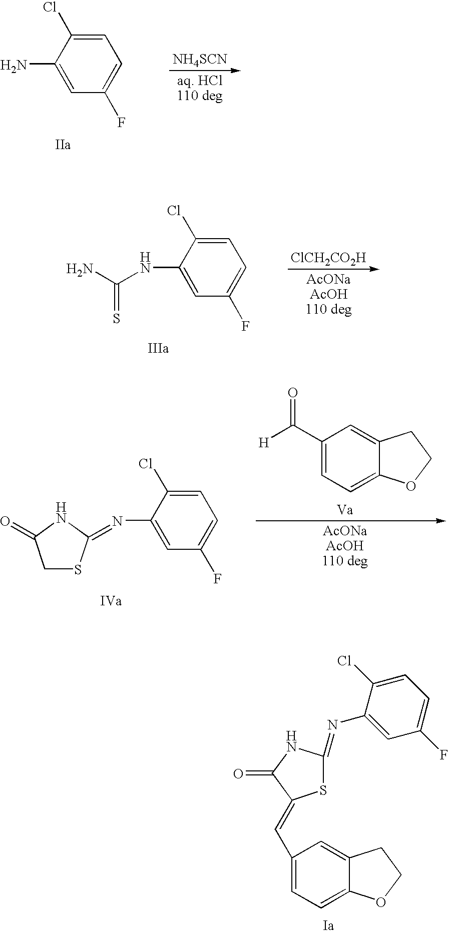

A mixture of 2-chloro-5-fluoroaniline IIa (2.0 g, 13.7 mmol) and 1.7 g of NH₄SCN in 4N—HCl (20 mL) was heated to reflux at 110 C° for 6 hours. After cooling, it was treated with H₂O to form a solid, followed by desiccation in vacuo to give thiourea IIIa (870 mg, 4.3 mmol). A mixture of IIIa (870 mg, 4.3 mmol), ClCH₂CO₂H (400 mg), and AcONa (350 mg) in AcOH (5 mL) was heated to reflux at 110 C° for 4 h. The mixture was poured onto water and the formed solid was isolated by filtration. It was washed with MeOH to give imino thiazolidinone IVa (456 mg, 1.9 mmol). A mixture of IVa (98 mg, 0.4 mmol), aldehyde Va (60 mg, 0.4 mmol), AcONa (100 mg) in AcOH (2 mL) was heated to reflux at 120 degree for 48 hours. After cooling, a small portion of water was added until the solid forms. It was filtered and washed with MeOH, followed by desiccation in vacuo to afford a target product Ia (61 mg, 0.16 mmol).

¹HNMR: (DMSO-d₆) δ 3.21 (t, 2H), 4.58 (t, 2H), 6.87 (d, 1H), 7.06 (sbr, 2H), 7.30 (d, 1H), 7.39 (s, 1H), 7.58 (sbr, 2H), 12.60 (sbr, 1H): LC/MS: m/z 375 (M+1), 377 (M+3)

Compounds in Examples 2-61, 73-94, and 96 were made by the process described in Scheme A, analogous to the method described in Example 1.

### Example 2

### 2-(2-Chloro-phenylimino)-5-(2-oxo-2H-chromen-6-ylmethylene)-thiazolidin-4-one

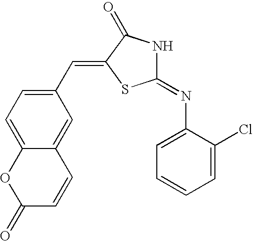

¹H NMR (DMSO-d₆) δ 6.52 (d, 1H), 7.15 (d, 1H), 7.21 (t, 1H), 7.38 (t, 1H), 7.49 (d, 1H), 7.54 (d, 1H), 7.72 (s, 1H), 7.71-7.74 (m, 1H), 7.85 (s, 1H), 8.13 (d, 1H), 12.73 (s br, 1H): LC/MS: m/z 383 (M+1), 385 (M+3)

### Example 3

### 2-(2-Chloro-phenylimino)-5-(2-oxo-2H-chromen-6-ylmethylene)-thiazolidin-4-one

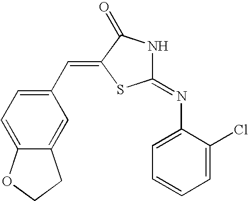

¹H NMR (DMSO-d₆) δ 3.19 (t, 2H), 4.58 (t, 2H), 6.87 (d, 1H), 71-6 (d, 1H), 7.20 (t, 1H), 7.28 (d, 1H), 7.37 (m, 2H), 7.54 (d, 1H), 7.61 (s, 1H), 12.54 (brs, 1H): LC/MS: m/z 357 (M+1), 359(M+3)

### Example 4

### 2-(2-Chloro-phenylimino)-5-(2-oxo-2H-chromen-6-ylmethylene)-thiazolidin-4-one

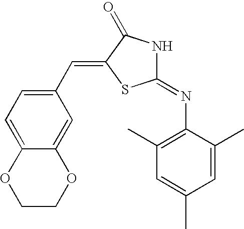

¹H NMR (DMSO-d₆) δ 2.06 (s, 6H), 2.25 (s, 3H), 4.24 (dd, 4H), 6.94 (m, 4H), 6.96 (s, 1H), 7.52 (s, 1H), 12.5 (brs, 1H): LC/MS: m/z 381 (M+1)

### Example 5

### 5-(2,3-Dihydro-benzofuran-5-ylmethylene)-2-(2,4,6-trimethyl-phenylimino)-thiazolidin-4-one

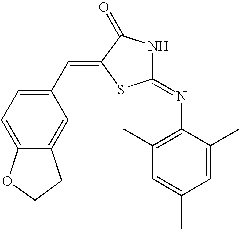

¹H NMR (DMSO-d₆) δ 2.05 (s, 6H), 2.24 (s, 3H), 3.19 (t, 2H), 4.56 (t, 2H), 6.84 (d, 1H), 6.91 (m, 2H), 7.22 (d, 1H), 7.31 (s, 1H), 7.51 (s, 1H), 12.5 (brs, 1H): LC/MS: m/z 365 (M+1)

### Example 6

### 2-Cyclohexylimino-5-(2,3-dihydro-benzo[1-6]dioxin-6-ylmethylene)-thiazolidin-4-one

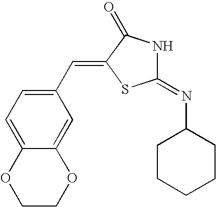

¹H NMR (DMSO-d₆) δ1.18 (sbr, 1H), 1.31 (mbr, 2H), 1.59 (dbr, 1H), 1.72 (sbr, 2H), 1.93 (sbr, 2H), 3.89 (brs, 1H), 6.99 (d, 1H), 7.05 (m, 2H), 7.48 (s, 1H), 9.50 (dbr, 1H): LC/MS: m/z 345 (M+1)

### Example 7

### 2-Cyclohexylimino-5-(2,3-dihydro-benzofuran-5-ylmethylene)-thiazolidin-4-one

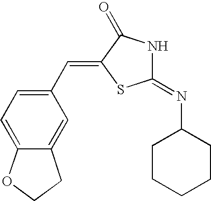

¹H NMR (DMSO-d₆) δ1.19 (mbr, 1H), 1.29 (mbr, 2H), 1.57 (dbr, 1H), 1.72 (sbr, 2H), 1.91 (mbr, 2H), 3.24 (t, 2H), 3.89 (sbr, 1H), 4.60 (t, 2H), 6.91 (d, 1H), 7.33 (d, 1H), 7.43 (s, 1H), 7.53 (s, 1H), 9.45 (d. 1H): LC/MS: m/z 329 (M+1)

### Example 8

### 5-Benzo[1,3]dioxol-5-ylmethylene-2-(2-chloro-phenylimino)-thiazolidin-4-one

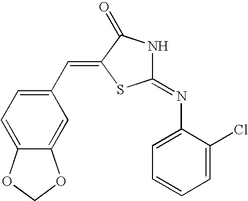

¹H NMR (DMSO-d₆) δ 6.08 (d, 2H), 7.03 (m, 2H), 7.07 (s, 1H), 7.13 (d, 1H), 7.19 (t, 1H), 7.36 (t, 1H), 7.53 (d, 1H), 7.58 (s, 1H), 12.54 (sbr, 1H): LC/MS: m/z 359 (M+1), 361 (M+3)

### Example 9

### 5-(2,3-Dihydro-benzofuran-5-ylmethylene)-2-o-tolylimino-thiazolidin-4-one

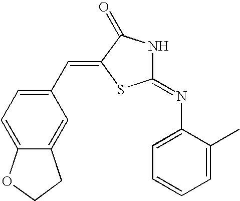

¹H NMR (DMSO-d₆) δ 2.14 (s, 3H), 3.19 (t, 2H), 4.57 (t, 2H), 6.86 (d, 1H), 6.93 (d, 1H), 7.10 (t, 1H), 7.22 (t, 1H), 7.27 (m, 2H), 7.35 (s, 1H), 7.57 (s, 1H), 12.24 (sbr, 1H): LC/MS: m/z 337 (M+1)

### Example 10

### 5-(2,3-Dihydro-benzo[1-6]dioxin-6-ylmethylene)-2-o-tolylimino-thiazolidin-4-one

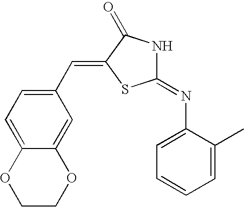

¹H NMR (DMSO-d₆) δ 2.14 (s, 3H), 4.23 (d, 2H), 4.26 (d, 2H), 6.96 (m, 2H), 7.00 (s, 1H), 7.11 (t, 1H), 7.22 (t, 1H), 7.29 (d, 1H), 7.53 (s, 1H), 12.29 (sbr, 1H): LC/MS: m/z 353 (M+1)

### Example 11

### 5-[2-(2-Chloro-phenylimino)-4-oxo-thiazolidin-5-ylidenemethyl]-3H-benzooxazol-2-one

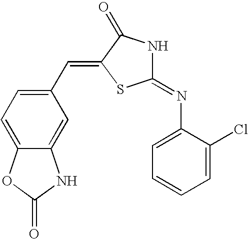

¹H NMR (DMSO-d₆) δ 7.14 (d, 1H), 7.18 (s, 1H), 7.20 (t, 1H), 7.28 (d, 1H), 7.38 (m, 2H), 7.54 (d, 1H), 7.69 (s, 1H), 12.10 (sbr, 1H): LC/MS: m/z 372 (M+1), 374 (M+3)

### Example 12

### 2-(2-Bromo-phenylimino)-5-(2,3-dihydro-benzofuran-5-ylmethylene)-thiazolidin-4-one

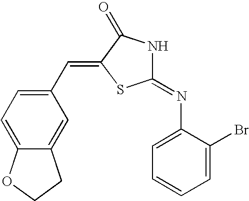

¹H NMR (DMSO-d₆) δ 3.19 (t, 2H), 4.57 (t, 2H), 6.87 (d, 1H), 7.11 (m, 2H), 7.28 (d, 1H), 7.36 (s, 1H), 7.40 (t, 1H), 7.60 (s. 1H), 7.69 (d, 1H), 12.51 (sbr, 1H): LC/MS: m/z 401 (M), 403 (M+2)

### Example 13

### 2-(2-Trifluoromethyl-phenylimino)-5-(2,3-dihydro-benzofuran-5-ylmethylene)-thiazolidin-4-one

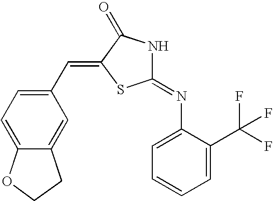

¹H NMR (DMSO-d₆) δ 3.19 (t, 2H), 4.58 (t, 2H), 6.87 (d, 1H), 7.22 (d, 1H), 7.29 (d, 1H), 7.36 (m, 2H), 7.62 (s, 1H), 7.69 (t, 1H), 7.75 (d, 1H), 12.58 (sbr, 1H): LC/MS: m/z 391 (M+1)

### Example 14

### 2-(2,6-Dichloro-phenylimino)-5-(2,3-dihydro-benzofuran-5-ylmethylene)-thiazolidin-4-one

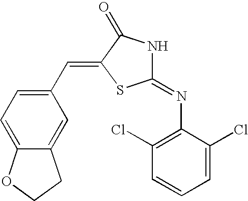

¹H NMR (DMSO-d₆) δ 3.20 (t, 2H), 4.58 (t, 2H), 6.87 (d, 1H), 7.20 (t, 1H), 7.28 (d, 1H), 7.36 (s, 1H), 7.55 (d, 1H), 7.64 (s, 1H), 12.77 (sbr, 1H): LC/MS: m/z 391 (M+1), 393 (M+3)

### Example 15

### 5-(2,3-Dihydro-benzofuran-5-ylmethylene)-2-(2-methylsulfanyl-phenylimino)-thiazolidin-4-one

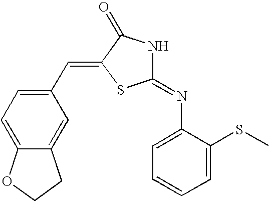

¹H NMR (DMSO-d₆) δ 2.38 (s, 3H), 3.19 (t, 2H), 4.57 (t, 2H), 6.85 (d, 1H), 6.93 (d, 1H), 7.17 (m, 2H), 7.25 (m, 2H), 7.35 (s, 1H), 7.52 (s, 1H), 12.32 (sbr, 1H): LC/MS: m/z 369 (M+1)

### Example 16

### 5-(2,3-Dihydro-benzofuran-5-ylmethylene)-2-(2-fluoro-phenyl i mino)-thiazolidin-4-one

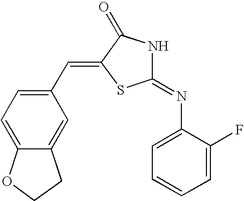

¹H NMR (DMSO-d₆) δ 3.20 (t, 2H), 4.58 (t, 2H), 6.88 (d, 1H), 7.15 (m, 1H), 7.21 (m, 2H), 7.29 (m, 2H), 7.38 (s, 1H), 7.61 (s, 1H): LC/MS: m/z 341 (M+1)

### Example 17

### 2-(2-Methylsulfanyl-phenylimino)-5-(quinolin-6-ylmethylene)-thiazolidin-4-one

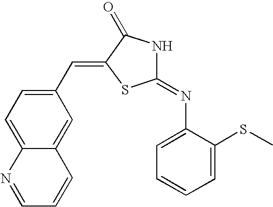

¹H NMR (DMSO-d₆) δ 2.40 (s, 3H), 6.99 (d, 1H), 7.17-7.30 (m, 3H), 7.56 (dd, 1H), 7.83 (m, 2H), 8.08 (d, 1H), 8.13 (s, 1H), 8.46 (d, 1H), 8.92 (m, 1H), 12.65 (sbr, 1H): LC/MS: m/z 378 (M+1)

### Example 18

### 2-(2-Bromo-phenylimino)-5-(quinolin-6-ylmethylene)-thiazolidin-4-one

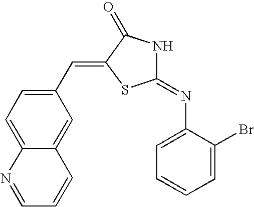

¹H NMR (DMSO-d₆) δ 7.15 (t, 2H), 7.43 (t, 1H), 7.56 (dd, 1H), 7.71 (d, 1H), 7.83 (s, 1H), 7.86 (s, 1H), 8.08 (d, 1H), 81-6 (s, 1H), 8.44 (d, 1H), 8.93 (m, 1H), 12.77 (brs, 1H): LC/MS: m/z 410 (M), 412 (M+2)

### Example 19

### 2-(2,3-Dimethyl-phenylimino)-5-(quinolin-6-ylmethylene)-thiazolidin-4-one

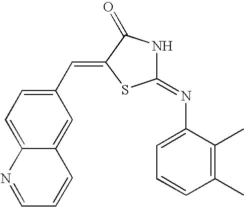

¹H NMR (DMSO-d₆) δ 2.07 (s, 3H), 2.27 (s, 3H), 6.81 (d, 1H), 7.03 (d, 1H), 7.12 (t, 1H), 7.55 (dd, 1H), 7.78 (s, 1H), 7.83 (dd, 1H), 8.06 (d, 1H), 8.11 (s, 1H), 8.42 (d, 1H), 8.92 (m, 1H): LC/MS: m/z 360 (M+1)

### Example 20

### 2-(Naphthalen-1-ylimino)-5-(quinolin-6-ylmethylene)-thiazolidin-4-one

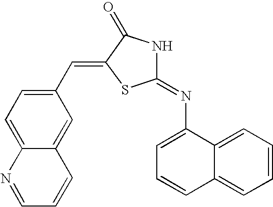

¹H NMR (DMSO-d₆) δ 7.17 (d, 1H), 7.54 (m, 4H), 7.80 (m, 2H), 7.82 (s, 1H), 7.97 (t, 2H), 8.03 (d, 1H), 8.09 (s, 1H), 8.38 (d, 1H), 8.90 (m, 1H): LC/MS: m/z 382 (M+1)

### Example 21

### 5-(Quinolin-6-ylmethylene)-2-(2-trifluoromethyl-phenylimino)-thiazolidin-4-one

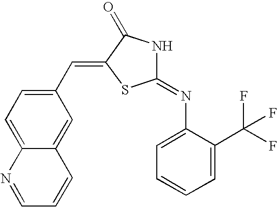

¹H NMR (DMSO-d₆) δ 7.23 (d, 1H), 7.36 (t, 1H), 7.55 (dd, 1H), 7.69 (t, 1H), 7.75 (d, 1H), 7.81 (s, 1H), 7.85 (d, 1H), 8.06 (d, 1H), 8.12 (s, 1H), 8.44 (d, 1H), 8.92 (d, 1H), 12.80 (sbr, 1H): LC/MS: m/z 400 (M+1)

### Example 22

### 2-(2-Chloro-5-trifluoromethyl-phenylimino)-5-quinolin-6-ylmethylene-thiazolidin-4-one

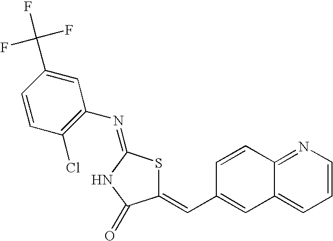

¹H NMR (DMSO-d₆) δ 7.50-7.60 (mbr, 2H), 7.56 (dd, 1H), 7.70-7.95 (mbr, 3H), 8.07 (d, 1H), 81-6 (s, 1H), 8.44 (d, 1H), 8.92 (m, 1H), 12.89 (sbr, 1H): LC/MS: m/z 434 (M+1), 436 (M+3)

### Example 23

### 2-(2,6-Dichloro-phenylimino)-5-8quinolin-6-ylmethylene)-thiazolidin-4-one

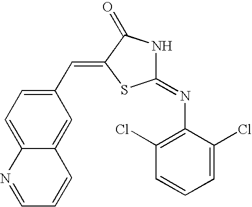

¹H NMR (DMSO-d₆) δ 7.23 (t, 1H), 7.55 (m, 3H), 7.84 (d, 1H), 7.87 (s, 1H), 8.08 (d, 1H), 81-6 (s, 1H), 8.46 (d, 1H), 8.93 (m, 1H), 13.01 (sbr, 1H): LC/MS: m/z 400 (M+1), 402 (M+3)

### Example 24

### 2-(2-Bromo-phenylimino)-5-(2,3-dihydro-benzo[1-6]dioxin-6-ylmethylene)-thiazolidin-4-one

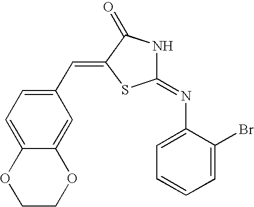

¹H NMR (DMSO-d₆) δ 4.25 (m, 4H), 6.97 (m, 3H), 7.13 (t, 2H), 7.42 (t, 1H), 7.57 (s, 1H), 7.70 (d, 1H), 12.60 (sbr, 1H): LC/MS: m/z 417 (M), 419 (M+2)

### Example 25

### 5-(Benzo[1,3]dioxol-5-ylmethylene)-2-(2-bromo-phenylimino)-thiazolidin-4-one

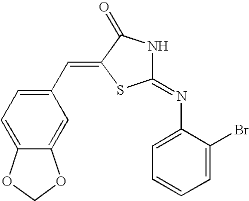

¹H NMR (DMSO-d₆) δ 6.09 (s, 2H), 7.03 (m, 3H), 7.13 (m, 2H), 7.41 (t, 1H), 7.60 (s, 1H), 7.69 (d, 1H), 12.60 (sbr, 1H) 403

### Example 26

### 2-(2-Chloro-phenylimino)-5-(quinoxalin-6-ylmethylene)-thiazolidin-4-one

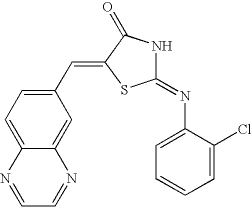

¹H NMR (DMSO-d₆) δ 7.19 (d, 1H), 7.23 (t, 1H), 7.39 (t, 1H), 7.56 (d, 1H), 7.92 (s, 1H), 7.98 (dd, 1H), 8.17 (m, 2H), 8.97 (s, 2H), 12.84 (sbr, 1H): LC/MS: m/z 367 (M+1), 369 (M+3)

### Example 27

### 2-(2,6-Dichloro-phenylimino)-5-(2,3-dihydro-benzo[1-6]dioxin-6-ylmethylene)-thiazolidin-4-one

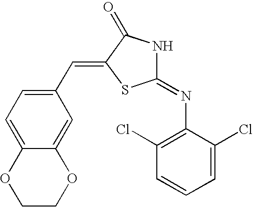

¹H NMR (DMSO-d₆) δ 4.25 (m, 4H), 6.97 (s, 2H), 7.02 (s, 1H), 7.22 (t, 1H), 7.55 (d, 2H), 7.60 (s, 1H), 12.84 (sbr, 1H): LC/MS: m/z 407 (M+1), 409 (M+3)

### Example 28

### 5-(2,3-Dihydro-benzo[1-6]dioxin-6-ylmethylene)-2-(2-nitro-phenylimino)-thiazolidin-4-one

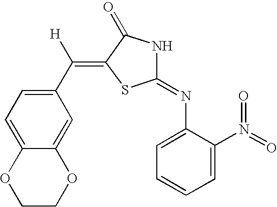

¹H NMR (DMSO-d₆) δ 4.26 (m, 4H), 6.96 (d, 1H), 7.03 (m, 2H), 7.31 (d, 1H), 7.38 (t, 1H), 7.58 (s, 1H), 7.72 8t, 1H), 8.01 (d, 1H), 12.66 (sbr, 1H): LC/MS: m/z 384 (M+1)

### Example 29

### 5-(2,3-Dihydro-benzofuran-5-ylmethylene)-2-(2-nitro-phenylimino)-thiazolidin-4-one

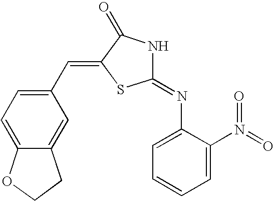

¹H NMR (DMSO-d₆) δ 3.20 (t, 2H), 4.58 (t, 2H), 6.88 (d, 1H), 7.30 (d, 2H), 7.39 (m, 2H), 7.64 (s, 1H), 7.73 (t, 1H), 8.03 (d, 1H), 12.63 (sbr, 1H): LC/MS: m/z 368 (M+1)

### Example 30

### 2-(2-Chloro-4-fluoro-5-methyl-phenylimino)-5-(2,3-dihydro-benzofuran-5-ylmethylene)-thiazolidin-4-one

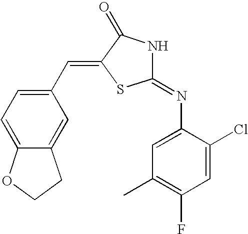

¹H NMR (DMSO-d₆) δ 2.22 (s, 3H), 3.20 (t, 2H), 4.58 (t, 2H), 6.87 (d, 1H), 7.05 (d, 1H), 7.28 (d, 1H), 7.38 (s, 1H), 7.44 (d, 1H), 7.58 (s, 1H), 12.43 (sbr, 1H): LC/MS: m/z 389 (M+1), 391 (M+3)

### Example 31

### 3-Chloro-4-[5-(2,3-dihydro-benzofuran-5-ylmethylene)-4-oxo-thiazolidin-2-ylideneamino]-benzoic acid methyl ester

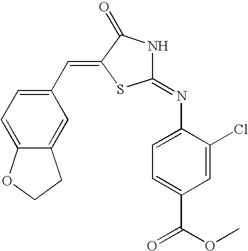

¹H NMR (DMSO-d₆) δ 3.20 (t, 2H), 3.87 (s, 3H), 4.57 (t, 2H), 6.85 (d, 1H), 7.29 (d, 1H), 7.38 (mbr, 2H), 7.52 (s, 1H), 7.88 (d, 1H), 7.99 (s, 1H), 12.4 (sbr, 1H): LC/MS: m/z 415 (M+1), 417 (M+3)

### Example 32

### 2-(2-Chloro-phenylimino)-5-(2,3-dihydro-benzo[1-6]dioxin-6-ylmethylene)-thiazolidin-4-one

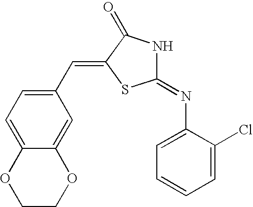

¹H NMR (DMSO-d₆) δ 4.25 (dd, 4H), 6.94-7.01 (m, 3H), 71-6 (d, 1H), 7.20 (t, 1H), 7.37 (t, 1H), 7.54 (d, 1H), 7.57 (s, 1H), 12.6 (s br, 1H): LC/MS: m/z 373 (M+1), 375 (M+3)

### Example 33

### 2-(2-Chloro-4-trifluoromethyl-phenylimino)-5-(2,3-dihydro-benzofuran-5-ylmethylene)-thiazolidin-4-one

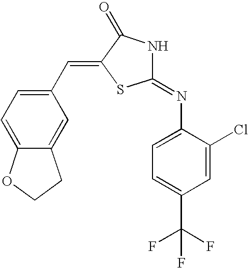

¹H NMR (DMSO-d₆) δ 3.20 (t, 2H), 4.58 (t, 2H), 6.87 (d, 1H), 7.30 (d, 1H), 7.37 (m, br), 7.40 (s, 1H), 7.62 (s, 1H), 7.73 (d, 1H), 7.95 (s, 1H), 12.68 (sbr, 1H): LC/MS: m/z 425 (M+1), 427 (M+3)

### Example 34

### 2-(4-Bromo-2-chloro-phenylimino)-5-(2,3-dihydro-benzofuran-5-ylmethylene)-thiazolidin-4-one

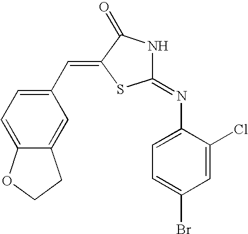

¹H NMR (DMSO-d₆) δ 3.20 (t, 2H), 4.57 (t, 2H), 6.85 (d, 1H), 7.07 (sbr, 1H), 7.28 (d, 1H), 7.37 (s, 1H), 7.51 (mbr, 2H), 7.76 (mbr, 1H), 12.07 (sbr, 1H): LC/MS: m/z 436 (M+1)

### Example 35

### 5-(2,3-Dihydro-benzofuran-5-ylmethylene)-2-(2-methanesulfinyl-phenylimino)-thiazolidin-4-one

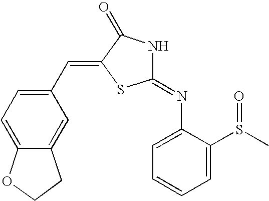

¹H NMR (DMSO-d₆) δ 2.68 (s, 3H), 3.20 (t, 2H), 4.58 (t, 2H), 6.87 (d, 1H), 7.18 (d, 1H), 7.31 (d, 1H), 7.39 (s, 1H), 7.46 (t, 1H), 7.57 (t, 1H), 7.63 (s, 1H), 7.80 (d, 1H): LC/MS: m/z 385 (M+1)

### Example 36

### 3-Chloro-4-[5-(2,3-dihydro-benzofuran-5-ylmethylene)-4-oxo-thiazolidin-2-ylideneamino]-benzoic acid

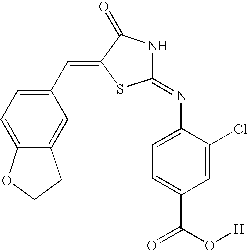

¹H NMR (DMSO-d₆) δ 3.20 (t, 2H), 4.55 (t, 2H), 6.82 (d, 1H), 7.25 (d, 1H), 7.28 (mbr, 2H), 7.36 (s, 1H), 7.73 (d, 1H), 7.86 (s, 1H): LC/MS: m/z 401 (M+1), 403 (M+3)

### Example 37

### 5-[2-(2-Chloro-phenylimino)-4-oxo-thiazolidin-5-ylidenemethyl]-1H-pyridin-2-one

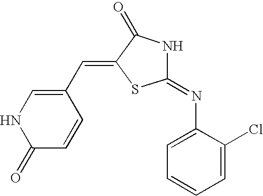

¹H NMR (DMSO-d₆) δ 6.40 (m, 1H), 7.07 (d, 1H), 7.13 (t, 1H), 7.32 (t, 1H), 7.38 (s, 1H), 7.50 (t, 2H), 7.77 (s, 1H), 12.07 (sbr, 1H): LC/MS: m/z 332 (M+1), 334 (M+3)

### Example 38

### 2-(2-Methylsulfanyl-phenylimino)-5-(quinolin-6-ylmethylene)-thiazolidin-4-one

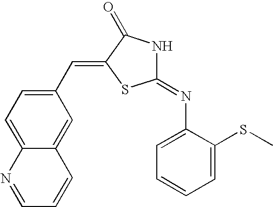

¹H NMR (DMSO-d₆) δ2.40 (s, 3H), 7.17-7.28 (m, 3H), 7.55 (dd, 1H), 7.80 (s, 1H), 7.84 (d, 1H), 8.07 (d, 1H), 8.12 (s, 1H), 8.42 (d, 1H), 8.92 (m, 1H), 12.56 (sbr, 1H): LC/MS: m/z 378 (M+1)

### Example 39

### 2-(2-Chloro-4-fluoro-5-methyl-phenylimino)-5-(quinolin-6-ylmethylene)-thiazolidin-4-one

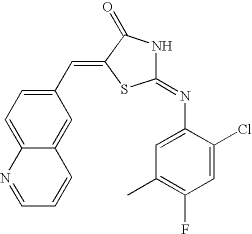

¹H NMR (DMSO-d₆) δ 2.23 (s, 3H), 7.10 (d, 1H), 7.48 (d, 1H), 7.57 (dd, 1H), 7.83 (s, 1H), 7.86 (dd, 1H), 8.08 (d, 1H), 81-6 (s, 1H), 8.46 (d, 1H), 8.93 (m, 1H), 12.69 (sbr, 1H): LC/MS: m/z 398 (M+1), 400 (M+3)

### Example 40

### 2-(2-Chloro-5-fluoro-phenylimino)-5-(quinolin-6-ylmethylene)-thiazolidin-4-one

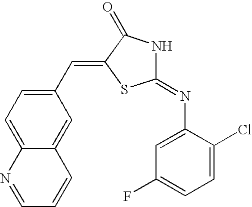

¹H NMR (DMSO-d₆) δ7.10 (sbr, 2H), 7.56 (dd, 1H), 7.58 (mbr, 1H), 7.82 (s, 1H), 7.88 (m, 1H), 8.07 (d, 1H), 81-6 (s, 1H), 8.46 (d, 1H), 8.93 (d, 1H), 12.81 (sbr, 1H): LC/MS: m/z 384 (M+1), 386 (M+3)

### Example 41

### 2-(2-Chloro-5-fluoro-phenylimino)-5-(2,3-dihydro-benzo[1-6]dioxin-6-ylmethylene)-thiazolidin-4-one

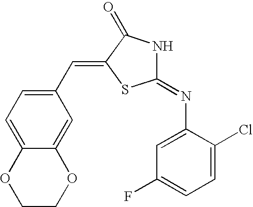

¹H NMR (DMSO-d₆) δ 4.26 (m, 4H), 6.95 (d, 1H), 7.02 (d, 1H), 7.05 (mbr, 3H), 7.55 (mbr, 2H), 12.65 (sbr, 1H): LC/MS: m/z 391 (M+1), 393 (M+3)

### Example 42

### 2-(2-Chloro-4-trifluoromethyl-phenylimino)-5-quinolin-6-ylmethylene-thiazolidin-4-one

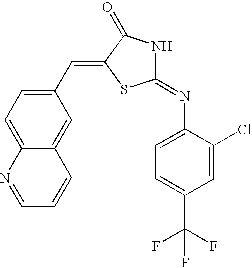

¹H NMR (DMSO-d₆) δ 7.41 (d, 1H), 7.57 (dd, 1H), 7.76 (d, 1H), 7.87 (m, 2H), 7.99 (s, 1H), 8.08 (d, 1H), 8.17 (s, 1H), 8.47 (d, 1H), 8.94 (dd, 1H), 12.90 (sbr, 1H): LC/MS: m/z 435 (M+1), 437 (M+3)

### Example 43

### 5-(Benzothiazol-6-ylmethylene)-2-(2-chloro-phenylimino)-thiazolidin-4-one

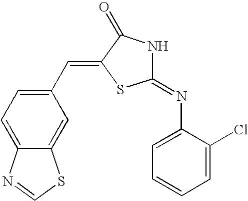

¹H NMR (DMSO-d₆) δ 7.14 (d, 1H), 7.20 (t, 1H), 7.37 (t, 1H), 7.53 (d, 1H), 7.65 (d, 1H), 7.77 (s, 1H), 81-6 (d, 1H), 8.36 (s, 1H), 9.47 (s, 1H), 12.61 (sbr, 1H): LC/MS: m/z 372 (M+1), 374 (M+3)

### Example 44

### 5-(Benzo[1,2,5]thiadiazol-5-ylmethylene)-2-(2-bromo-phenylimino)-thiazolidin-4-one

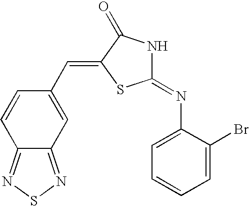

¹H NMR (DMSO-d₆) δ 7.15 (m, 2H), 7.43 (t, 1H), 7.71 (d, 1H), 7.83 (dd, 1H), 7.89 (s, 1H), 8.16 (d, 1H), 8.22 (s, 1H), 12.83 (sbr, 1H): LC/MS: m/z 417 (M), 419 (M+2)

### Example 45

### 5-(Benzo[1,2,5]thiadiazol-5-ylmethylene)-2-(2-chloro-5-fluoro-phenylimino)-thiazolidin-4-one

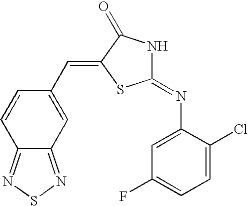

¹H NMR (DMSO-d₆) δ7.11 (m, 2H), 7.60 (t, 1H), 7.85 (d, 1H), 7.89 (s, 1H), 8.16 (d, 1H), 8.25 (s, 1H), 12.89 (sbr, 1H): LC/MS: m/z 391 (M+1), 393 (M+3)

### Example 46

### 5-(Benzothiazol-6-ylmethylene)-2-(2,6-dichloro-phenylimino)-thiazolidin-4-one

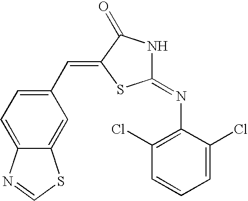

¹H NMR (DMSO-d₆) δ 7.23 (t, 1H), 7.57 (d, 2H), 7.66 (d, 1H), 7.86 (s, 1H), 8.15 (d, 1H), 8.39 (s, 1H), 9.49 (s, 1H), 12.98 (sbr, 1H): LC/MS: m/z 406 (M+1), 408 (M+3)

### Example 47

### 2-(2-Chloro-phenylimino)-5-(4-hydroxy-3-nitro-benzylidene)-thiazolidin-4-one

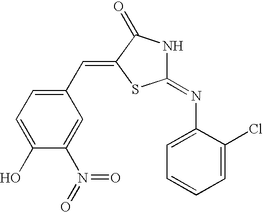

¹H NMR (DMSO-d₆) δ 7.14 (d, 1H), 7.22 (m, 2H), 7.38 (t, 1H), 7.54 (d, 1H), 7.62 (d, 1H), 7.67 (s, 1H), 8.08 (s, 1H), 11.75 (sbr, 1H), 12.69 (sbr, 1H): LC/MS: m/z 376 (M+1), 378 (M+3)

### Example 48

### 2-(2-Chloro-phenylimino)-5-(4-hydroxy-3-methoxy-benzylidene)-thiazolidin-4-one

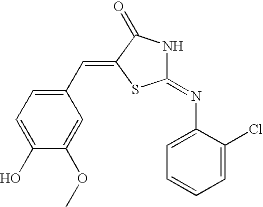

¹H NMR (DMSO-d₆) δ 3.75 (s, 3H), 6.88 (m, 2H), 7.15 (t, 1H), 7.19 (t, 1H), 7.36 (t, 1H), 7.53 (d, 1H), 7.58 (s, 1H), 9.80 (sbr, 1H), 12.30 (sbr, 1H): LC/MS: m/z 361 (M+1), 363 (M+3)

### Example 49

### 2-(2-Chloro-phenylimino)-5-(4-hydroxy-2-methoxy-benzylidene)-thiazolidin-4-one

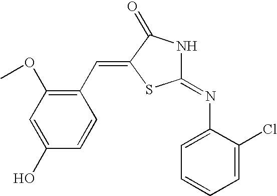

¹H NMR (DMSO-d₆) δ 3.81 (s, 3H), 6.47 (m, 2H), 7.10 (m, 2H), 7.19 (t, 1H), 7.35 (t, 1H), 7.53 (d, 1H), 7.83 (s, 1H), 10.30 (sbr, 1H), 12.21 (sbr, 1H) 360

### Example 50

### 2-(2-Chloro-phenylimino)-5-(4-hydroxy-benzylidene)-thiazolidin-4-one

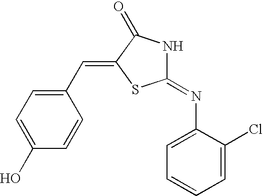

¹H NMR (DMSO-d₆) δ 6.86 (d, 2H), 7.13 (d, 1H), 7.20 (t, 1H), 7.34 (d, 2H), 7.36 (m, 1H), 7.53 (d, 1H), 7.58 (s, 1H), 10.20 (sbr, 1H), 12.48 (sbr, 1H): LC/MS: m/z 331 (M+1), 333 (M+3)

### Example 51

### 2-(2-Chloro-phenylimino)-5-(4-methoxy-benzylidene)-thiazolidin-4-one

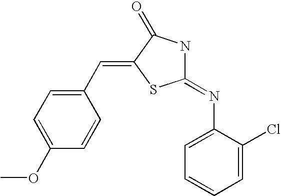

¹H NMR (DMSO-d₆) δ 3.78 (s, 3H), 7.05 (d, 2H), 71-6 (m, 1H), 7.21 (t, 1H), 7.37 (t, 1H), 7.46 (d, 2H), 7.54 (d, 1H), 7.63 (s, 1H), 12.54 (sbr, 1H): LC/MS: m/z 345 (M+1), 347 (M+3)

### Example 52

### 5-(3-Chloro-4-hydroxy-benzylidene)-2-(2-chloro-phenylimino)-thiazolidin-4-one

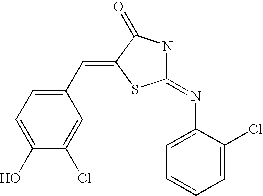

¹H NMR (DMSO-d₆) δ 7.06 (d, 1H), 71-6 (d, 1H), 7.21 (t, 1H), 7.28 (d, 1H), 7.37 (t, 1H), 7.55 (m, 3H), 11.02 (sbr, 1H), 12.0 (sbr, 1H): LC/MS: m/z 365 (M+1), 367 (M+3)

### Example 53

### 2-(2-Chloro-phenylimino)-5-(3-fluoro-4-methoxy-benzylidene)-thiazolidin-4-one

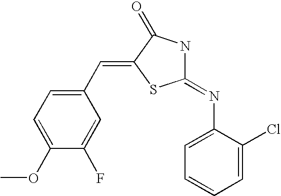

¹H NMR (DMSO-d₆) δ 7.13 (d, 1H), 7.19 (t, 1H), 7.28 (m, 2H), 7.36 (t, 1H), 7.40 (d, 1H), 7.53 (d, 1H), 7.58 (s, 1H), 12.59 (sbr, 1H) 362

### Example 54

### 2-(2,6-Dichloro-phenylimino)-5-(3-fluoro-4-hydroxy-benzylidene)-thiazolidin-4-one

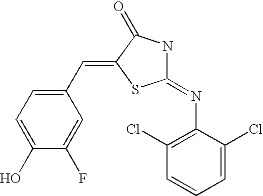

¹H NMR (DMSO-d₆) δ 7.03 (t, 1H), 7.12 (mbr, 2H), 7.30 (d, 1H), 7.50 (mbr, 3H), 12.08 (sbr, 1H): LC/MS: m/z 383 (M+1), 385 (M+3)

### Example 55

### 2-(2-Chloro-phenylimino)-5-(3-fluoro-4-hydroxy-benzylidene)-thiazolidin-4-one

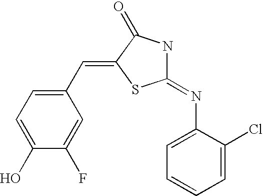

¹H NMR (DMSO-d₆) δ 7.05 (t, 1H), 71-6 (d, 1H), 7.21 (t, 1H), 7.37 (m, 2H), 7.54 (d, 1H), 7.58 (s, 1H), 10.67 (sbr, 1H), 12.11 (sbr, 1H): LC/MS: m/z 349 (M+1), 351 (M+3)

### Example 56

### 2-(2-Chloro-5-fluoro-phenylimino)-5-(3-fluoro-4-hydroxy-benzylidene)-thiazolidin-4-one

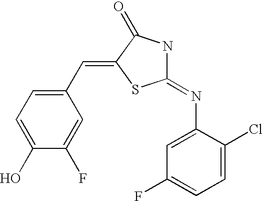

¹H NMR (DMSO-d₆) δ 7.04-7.13 (m, 3H), 7.17 (d, 1H), 7.39 (d, 1H), 7.60 (m, 2H), 10.69 (sbr, 1H), 12.00 (sbr, 1H): LC/MS: m/z 367 (M+1), 369 (M+3)

### Example 57

### 5-(3-Fluoro-4-hydroxy-benzylidene)-2-o-tolylimino-thiazolidin-4-one

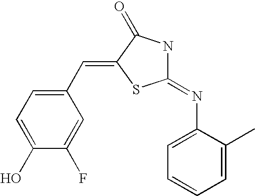

¹H NMR (DMSO-d₆) δ 21-6 (s, 3H), 6.94 (d, 1H), 7.04 (t, 1H), 7.12 (m, 2H), 7.23 (t, 1H), 7.28 (d, 1H), 7.33 (d, 1H), 7.54 (s, 1H), 10.66 (sbr, 1H), 12.12 (sbr, 1H): LC/MS: m/z 329 (M+1)

### Example 58

### 2-(2-Chloro-phenylimino)-5-quinolin-6-ylmethylene-thiazolidin-4-one

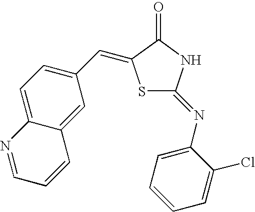

1H NMR (400 MHz, DMSO-d₆) ppm 7.17-7.25 (m, 2H), 7.39 (m, 1H), 7.57 (m, 2H), 7.84 (m, 1H), 7.86 (s, 1H), 8.08 (d, 1H, J=8.8 Hz), 81-6 (s, 1H), 8.45 (d, 1H, J=7.8 Hz), 8.93 (m, 1H). LC/MS: m/z 366 (M+1)+, 364 (M−1)−.

### Example 59

### 5-Quinolin-6-ylmethylene-2-(2,4,6-trimethyl-phenylimino)-thiazolidin-4-one

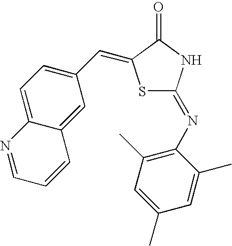

1H NMR (400 MHz, DMSO-d6) ppm 2.15 (s, 6H), 2.27 (s, 3H), 6.95 (s, 2H), 7.56 (m, 1H), 7.81 (m, 2H), 8.07 (d, 1H, J=8.8 Hz), 8.11 (s, 1H), 8.42 (d, 1H, J=8.4 Hz), 8.92 (m, 1H). LC/MS: m/z 374 (M+1)+, 372 (M−1)−.

### Example 60

### 5-Quinolin-6-ylmethylene-2-o-tolylimino-thiazolidin-4-one

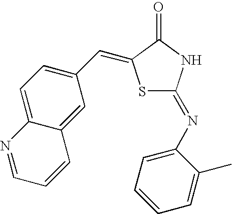

1H NMR (400 MHz, DMSO-d6) ppm 2.17 (s, 3H), 6.98 (m, 1H), 71-6 (m, 1H), 7.22-7.31 (m, 2H), 7.56 (m, 1H), 7.81 (s, 1H), 7.83 (m, 1H), 8.07 (d, 1H, J=8.8 Hz), 8.12 (s, 1H), 8.42 (d, 1H, J=7.6 Hz), 8.92 (m, 1H), 12.47 (m, 1H). LC/MS: m/z 346 (M+1)+, 344 (M−1)−.

### Example 61

### 2-(2-Methoxy-phenylimino)-5-quinolin-6-ylmethylene-thiazolidin-4-one

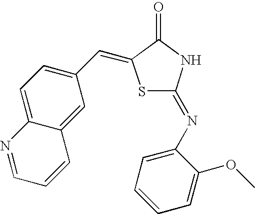

A mixture of E, Z-isomers (ratio=3.0/1.0)

1H NMR (400 MHz, DMSO-d₆) ppm 3.78 (s, 2.25H), 3.90 (s, 0.75H), 6.97-7.28 (m, 3H), 7.56 (m, 0.75H), 7.62 (m, 0.25H), 7.81-7.86 (m, 2H), 7.94-8.24 (m, 3H), 8.42-8.51 (m, 1H), 8.92 (m, 0.75H), 8.96 (m, 0.25H), 12.44 (m, 1H). LC/MS: m/z 362 (M+1)+, 360 (M−1)−.

### Example 62

### 5-(2,3-Dihydro-benzofuran-5-ylmethylene)-2-(2-dimethylamino-ethylamino)-thiazol-4-one

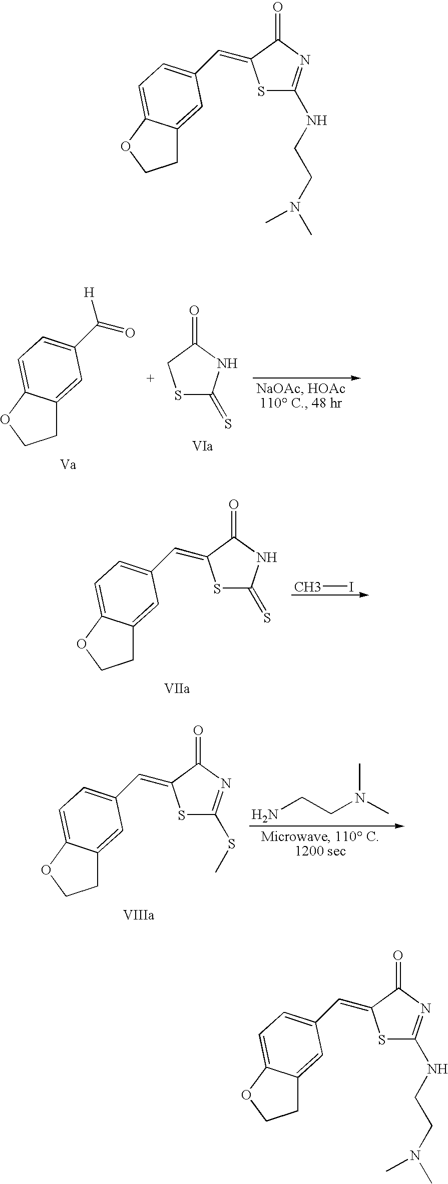

A mixture of aldehyde of formula Va (10 mmol), Rhodanine VIa (10 mmol), sodium acetate (30 mmol), and 10 mL of acetic acid was heated at 110 C° for 48 h. The reaction mixture was cooled to room temperature and filtered to collect the precipitate formed. The precipitate was washed with acetic acid (1 mL), methanol (1 mL) and dried in vaccuo to give compound VIIa 3.9 g (14.81 mmol).

To room a temperature suspension of VIIa (14.81 mmol) in 100 mL ethanol was added Hunig's base (5.2 mL, 29.85 mmol) followed by iodomethane (4.6 mL, 73.9 mmol).

After stirring the resultant suspension at room temperature for 3.5 h, the precipitate was filtered and washed with water to afford compound VIIIa 3.12 g (11.25 mmol) as a first crop. After evaporating the filtrate, to the residue was added methanol (10 mL) and water (10 mL), and the resultant mixture was subjected to sonication for 1 min. The process yielded the second crop which was filtered. 0.8 g (2.89 mmol).

To a mixture of VIIIa (0.3 mmol) and MS4A (molecular sieve 4 Angstrom powder) (250 mg) was added dimethylaminoethylamine (0.45 mmol) and ethanol (1 mL, dehydrated). The mixture was heated by microwave (SmithSynthesizer-Personal Chemistry) at 110 C° for 1200 seconds. The corresponding product was obtained in 65% yield after purification on SCX column.

¹H NMR (400 MHz, DMSO-d₆) ppm 2.18 (s, 6H), 2.44 (t, 2H, J=6.6 Hz), 3.24 (t, 2H, J=8.6 Hz), 3.58 (t, 2H, J=6.6 Hz), 4.60 (t, 2H, J=8.6 Hz), 6.90 (d, 1H, J=8.3 Hz), 7.30-7.48 (m, 3H). LC/MS: m/z 318 (M+1)+, 316 (M−1)−.

Example 63-72 compounds were made according to the process B, analogous to the method described in Example 62.

### Example 63

### Benzoic acid N′-(4-oxo-5-quinolin-6-ylmethylene-4,5-dihydro-thiazol-2-yl)-hydrazide

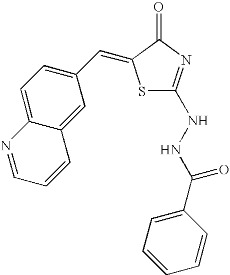

1H NMR (400 MHz, DMSO-d₆) ppm 7.49-7.63 (m, 4H), 7.84 (s, 1H), 7.91-7.97 (m, 3H), 8.12 (d, 1H, J=8.8 Hz), 8.23 (d, 1H, J=2.0 Hz), 8.48 (d, 1H, J=7.8 Hz), 8.95 (m, 1H), 11.17 (s, 1H), 12.63 (br, 1H). LC/MS: m/z 375 (M+1)+, 373 (M−1)−.

### Example 64

### 2-(2-Dimethylamino-ethylimino)-5-quinolin-6-ylmethylene-thiazolidin-4-one

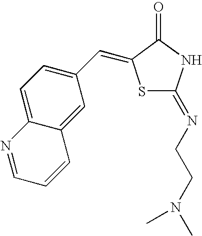

1H NMR (400 MHz, CD₃OD) ppm 2.80 (s, 6H), 3.24 (t, 2H, J=6.0 Hz), 3.94 (t, 2H, J=6.0 Hz), 7.57 (m, 1H), 7.88-7.91 (m, 2H), 8.04-8.08 (m, 2H), 8.37-8.45 (m, 2H), 8.86 (dd, 1H, J=1.8, 4.6 Hz). LC/MS: m/z 327 (M+1)+, 325 (M−1)−.

### Example 65

### 5-(2,3-Dihydro-benzofuran-5-ylmethylene)-2-(piperidin-1-ylamino)-thiazol-4-one

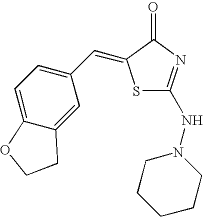

1H NMR (400 MHz, DMSO-d₆) ppm 1-60 (br, 2H), 1.63 (m, 4H), 2.27 (m, 4H), 3.26 (t, 2H, J=8.6 Hz), 4.61 (t, 2H, J=8.6 Hz), 6.93 (d, 1H, J=8.4 Hz), 7.37 (dd, 1H, J=1.8, 8.4 Hz), 7.47 (s, 1H), 7.51 (s, 1H), 11.68 (br, 1H). LC/MS: m/z 330 (M+1)+, 328 (M−1)−.

### Example 66

### 2-Benzylamino-5-(2,3-dihydro-benzofuran-5-ylmethylene)-thiazol-4-one

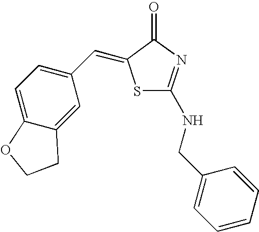

1H NMR (400 MHz, DMSO-d₆) ppm 3.25 (t, 2H, J=8.6 Hz), 4.60 (t, 2H, J=8.6 Hz), 4.73 (s, 2H), 6.92 (d, 1H, J=8.4 Hz), 7.29-7.57 (m, 8H), 9.97 (br, 1H). LC/MS: m/z 337 (M+1)+, 335 (M−1)−.

### Example 67

### 2-(4-tert-Butyl-thiazol-2-ylamino)-5-(2,3-dihydro-benzofuran-5-ylmethylene)-thiazol-4-one

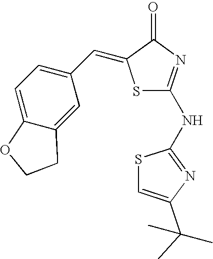

1H NMR (400 MHz, DMSO-d₆) ppm 1.35 (s, 9H), 3.24 (t, 2H, J=8.6 Hz), 4.64 (t, 2H, J=8.6 Hz), 6.93 (d, 1H, J=8.3 Hz), 7.02 (s, 1H), 7.46 (dd, 1H, J=1.8, 8.3 Hz), 7.57 (br, 1H), 7.65 (s, 1H), 12.53 (s, 1H). LC/MS: m/z 386 (M+1)+, 384 (M−1)−.

### Example 68

### 4-{[5-(2,3-Dihydro-benzofuran-5-ylmethylene)-4-oxo-4,5-dihydro-thiazol-2-ylamino]-methyl}-benzenesulfonamide

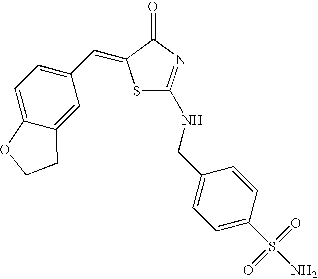

### Example 69

### 5-(2,3-Dihydro-benzofuran-5-ylmethylene)-2-(3-dimethylamino-propylamino)-thiazol-4-one

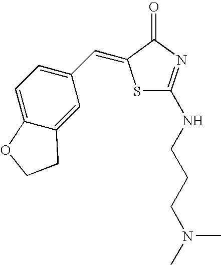

1H NMR (400 MHz, DMSO-d₆) ppm 1.74 (m, 2H), 2.13 (s, 6H), 2.25 (t, 2H, J=6.8 Hz), 3.24 (t, 2H, J=8.6 Hz), 3.51 (t, 2H, J=6.8 Hz), 4.61 (t, 2H, J=8.6 Hz), 6.91 (d, 1H, J=8.3 Hz), 7.57-7.52 (m, 3H). LC/MS: m/z 332 (M+1)+, 330 (M−1)−.

### Example 70

### 5-(2,3-Dihydro-benzofuran-5-ylmethylene)-2-(3-imidazol-1-yl-propylamino)-thiazol-4-one

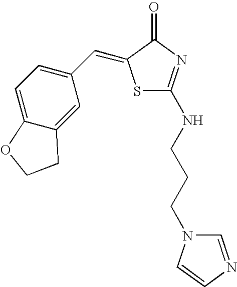

1H NMR (400 MHz, DMSO-d₆) ppm 2.04 (m, 2H), 3.25 (t, 2H, J=8.8 Hz), 3.45 (t, 2H, J=7.0 Hz), 4.04 (t, 2H, J=7.0 Hz), 4.61 (t, 2H, J=8.8 Hz), 6.91 (s, 1H), 6.92 (d, 1H, J=8.6 Hz), 7.22 (t, 1H, J=1.3 Hz), 7.34 (dd, 1H, J=1.5, 8.3 Hz), 7.43 (s, 1H), 7.55 (s, 1H), 7.66 (m, 1H), 9.57 (br, 1H). LC/MS: m/z 355 (M+1)+, 353 (M−1)−.

### Example 71

### Phenyl-Carbamic Acid

### N′-[5-(2,3-dihydro-benzofuran-5-ylmethylene)-4-oxo-4,5-dihydro-thiazol-2-yl]-hydrazide

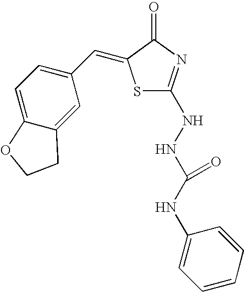

1H NMR (400 MHz, DMSO-d₆) ppm 3.26 (t, 2H, J=8.8 Hz), 4.62 (t, 2H, J=8.8 Hz), 6.93-7.01 (m, 2H), 7.24-7.62 (m, 6H), 9.17 (s, 1H). LC/MS: m/z 381 (M+1)+, 379 (M−1)−.

### Example 72

### Benzoic acid N′-[5-(2,3-dihydro-benzofuran-5-ylmethylene)-4-oxo-4,5-dihydro-thiazol-2-yl]-hydrazide

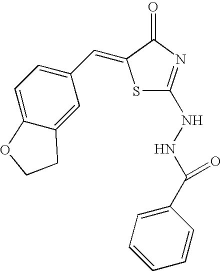

1H NMR (400 MHz, DMSO-d₆) ppm 3.23 (t, 2H, J=8.6 Hz), 4.60 (t, 2H, J=8.6 Hz), 6.91 (d, 1H, J=8.3 Hz), 7.37 (dd, 1H, J=1.5, 8.3 Hz), 7.47-7.61 (m, 5H), 7.90 (d, 2H, J=7.3 Hz), 11.08 (s, 1H), 12.49 (br, 1H). LC/MS: m/z 355 (M+1)+, 353 (M−1)−.

### Example 73

### 5-Benzo[1,2,5]thiadiazol-5-ylmethylene-2-(2,3,4-trifluoro-phenylamino)-thiazol-4-one

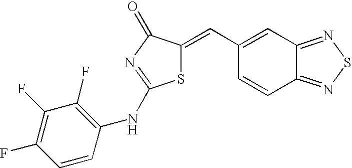

¹H NMR (DMSO-d₆) δ 7.07 (m, 1H), 7.37 (q, 1H), 7.86 (dd, 1H), 7.90 (s, 1H), 8.17 (d, 1H), 8.25 (d, 1H), 12.84 (s, 2H): LC/MS: m/z 393 (M+1).

### Example 74

### 5-Benzo[1,2,5]oxadiazol-5-ylmethylene-2-(2-nitro-phenylamino)-thiazol-4-one

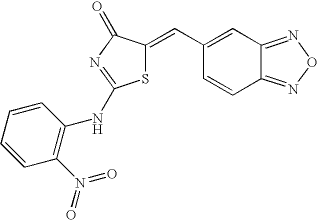

¹H NMR (DMSO-d₆) δ 7.33 (d, 1H), 7.40 (t, 1H), 7.73 (m, 2H), 7.81 (s, 1H), 8.04 (d, 1H), 8.12 (d, 1H), 8.18 (s, 1H), 12.97 (sbr, 1H): LC/MS: m/z 368 (M+1).

### Example 75

### 2-(2,6-Dichloro-phenylamino)-5-(4-[1,2,4]triazol-1-yl-benzylidene)-thiazol-4-one

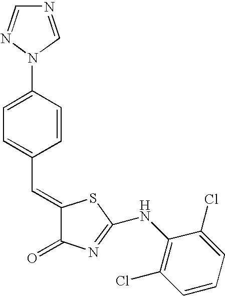

¹H NMR (DMSO-d₆) δ 7.23 (t, 1H), 7.57 (d, 1H), 7.69 (d, 1H), 7.78 (s, 1H), 7.97 (d, 1H), 8.27 (s, 1H), 9.34 (s, 1H), 12.99 (sbr, 1H): LC/MS: m/z 416 (M+1).

### Example 76

### 2-(2,6-Dichloro-phenylamino)-5-(1H-pyrrolo[2,3-b]pyridin-2-ylmethylene)-thiazol-4-one

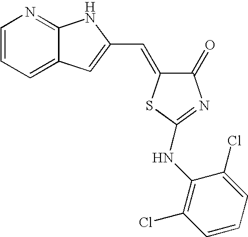

¹H NMR (DMSO-d₆) δ 7.20-7.24 (m, 2H), 7.56 (d, 2H), 7.68 (s, 1H), 7.97 (s, 1H), 8.34 (m, 2H), 12.53 (s, 1H), 12.65 (sbr, 1H): LC/MS: m/z 389 (M+1), 391 (M+3)

### Example 77

### 5-Benzo[1,2,5]thiadiazol-5-ylmethylene-2-(2,6-dichloro-phenylamino)-thiazol-4-one

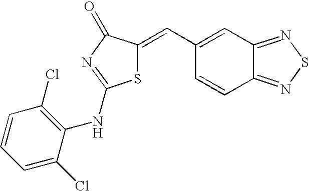

¹H NMR (DMSO-d₆) δ 7.24 (t, 1H), 7.57 (d, 2H), 7.81 (d, 1H), 7.95 (s, 1H), 8.16 (d, 1H), 8.25 (s, 1H), 13.10 (sbr, 1H): LC/MS: m/z 407 (M+1), 409 (M+3).

### Example 78

### 5-[2-(2-Methoxy-6-methyl-phenylamino)-4-oxo-4H-thiazol-5-ylidenemethyl]-1H-pyridin-2-one

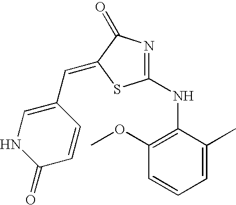

¹H NMR (DMSO-d₆) δ 2.09 (s, 1H), 3.72 (s, 3H), 6.40 (d, 1H), 6.86 (d, 1H), 6.92 (d, 1H), 7.08 (t, 1H), 7.42 (s, 1H), 7.45 (dd, 1H), 7.78 (s, 1H), 12.04 (sbr, 1H): LC/MS: m/z 342 (M+1)

### Example 79

### 5-Benzo[1,2,5]thiadiazol-5-ylmethylene-2-(2-nitro-phenylamino)-thiazol-4-one

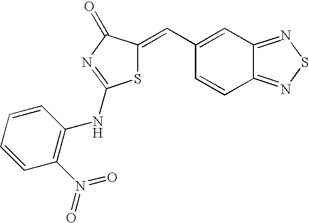

¹H NMR (DMSO-d₆) δ 7.34 (d, 1H), 7.40 (mbr, 1H), 7.73 (t, 1H), 7.86 (d, 1H), 7.90 (sbr, 1H), 8.03 (d, 1H), 8.16 (d, 1H), 8.24 (d, 1H), 11.98 (sbr, 1H): LC/MS: m/z 384 (M+1).

### Example 80

### 2-(2-Bromo-6-fluoro-phenylamino)-5-quinolin-6-ylmethylene-thiazol-4-one

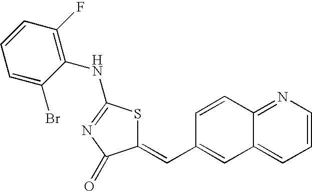

¹H NMR (DMSO-d₆) δ 7.18 (q, 1H), 7.39 (t, 1H), 7.55 (t, 1H), 7.58 (s, 1H), 7.85 (d, 1H), 7.86 (s, 1H), 8.07 (d, 1H), 81-6 (s, 1H), 8.46 (d, 1H), 8.93 (d, 1H), 12.98 (sbr, 1H): LC/MS: m/z 428 (M), 430 (M+2).

### Example 81

### 2-(2-Methoxy-6-methyl-phenylamino)-5-quinolin-6-ylmethylene-thiazol-4-one

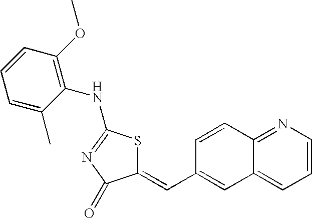

¹H NMR (DMSO-d₆) δ 2.10 (s, 3H), 3.72 (s, 3H), 6.87 (d, 1H), 6.92 (d, 1H), 7.07 (t, 1H), 7.54 (dd, 1H), 7.69 (s, 1H), 7.81 (d, 1H), 8.04 (d, 1H), 8.06 (s, 1H), 8.40 (d, 1H), 8.90 (d, 1H), 12.02 (sbr, 1H): LC/MS: m/z 376 (M+1).

### Example 82

### 5-Quinolin-6-ylmethylene-2-(2,3,4-trifluoro-phenylamino)-thiazol-4-one

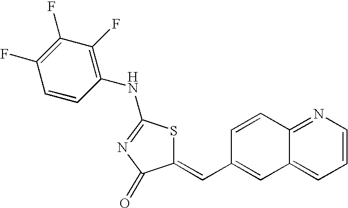

¹H NMR (DMSO-d₆) δ 7.06 (m, 1H), 7.37 (q, 1H), 7.58 (q, 1H), 7.84 (s, 1H), 7.88 (d, 1H), 8.08 (d, 1H), 8.15 (s, 1H), 8.46 (d, 1H), 8.93 (m, 1H), 11.99 (sbr, 1H): LC/MS: m/z 386 (M+1).

### Example 83

### 2-(2,6-Dichloro-phenylamino)-5-(2-oxo-2H-chromen-6-ylmethylene)-thiazol-4-one

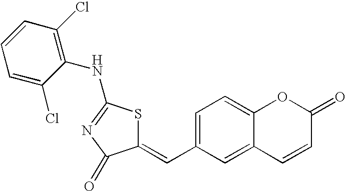

¹H NMR (DMSO-d₆) δ 6.52 (d, 1H), 7.23 (t, 1H), 7.49 (d, 1H), 7.56 (d, 2H), 7.73 (d, 1H), 7.77 (s, 1H), 7.85 (s, 1H), 8.15 (d, 1H), 12.99 (sbr, 1H): LC/MS: m/z 417 (M+1), 419 (M+3).

### Example 84

### 2-(2-Bromo-phenylamino)-5-(5-pyridin-2-yl-thiophen-2-ylmethylene)-thiazol-4-one

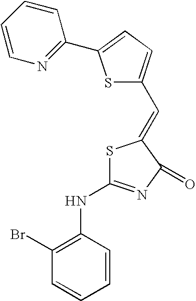

¹H NMR (DMSO-d₆) δ 7.17 (t, 2H), 7.32 (dd, 1H), 7.46 (t, 1H), 7.63 (d, 1H), 7.72 (d, 1H), 7.86 (t, 1H), 7.91 (m, 2H), 7.99 (d, 1H), 8.54 (d, 1H), 12.65 (sbr, 1H): LC/MS: m/z 442 (M), 444 (M+2).

### Example 85

### 2-(2-Bromo-phenylamino)-5-(1-oxy-pyridin-4-ylmethylene)-thiazol-4-one

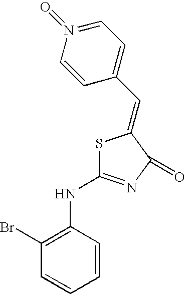

¹H NMR (DMSO-d₆) δ 7.14 (t, 2H), 7.42 (t, 1H), 7.49 (d, 2H), 7.63 (s, 1H), 7.70 (d, 1H), 8.22 (d, 2H), 12.82 (sbr, 1H): LC/MS: m/z 376 (M), 378 (M+2).

### Example 86

### 2-(2-Bromo-phenylamino)-5-(3-p-tolyl-benzo[c] isoxazol-5-ylmethylene)-thiazol-4-one

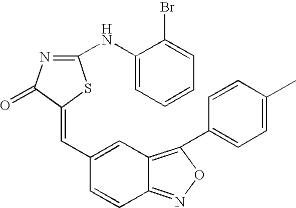

¹H NMR (DMSO-d₆) δ 2.44 (t, 3H), 7.15 (m, 2H), 7.42 (m, 3H), 7.51 (d, 1H), 7.72 (t, 2H), 7.81 (s, 1H), 8.02 (d, 2H), 8.45 (s, 1H), 12.73 (sbr, 1H): LC/MS: m/z 490 (M), 492 (M+2).

### Example 87

### 2-(2-Bromo-phenylamino)-5-(3,4-dihydro-2H-benzo[b][1-6]dioxepin-7-ylmethylene)-thiazol-4-one

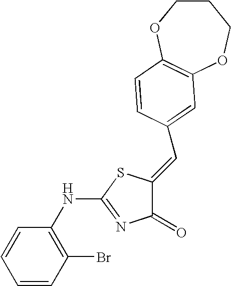

¹H NMR (DMSO-d₆) δ 2.10 (m, 2H), 4.16 (quint, 4H), 7.03 (m, 1H), 7.08-7.15 (m, 4H), 7.42 (t, 1H), 7.57 (s, 1H), 7.70 (d, 1H), 12.59 (sbr, 1H): LC/MS: m/z 431 (M), 433 (M+2).

### Example 88

### 5-Benzo[1,2,5]oxadiazol-5-ylmethylene-2-(2-bromo-phenylamino)-thiazol-4-one

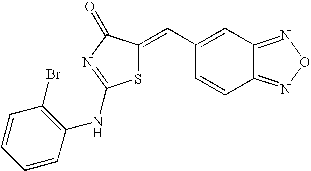

¹H NMR (DMSO-d₆) δ 7.13 (m, 2H), 7.41 (t, 1H), 7.71 (t, 2H), 7.76 (s, 1H), 8.11 (d, 1H), 8.13 (s, 1H), 12.92 (sbr, 1H): LC/MS: m/z 401 (M), 403 (M+2).

### Example 89

### 2-(2,6-Dichloro-phenylamino)-5-(2-methoxy-pyridin-3-ylmethylene)-thiazol-4-one

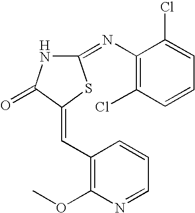

¹H NMR (DMSO-d₆) δ 3.88 (s, 3H), 6.91 (d, 1H), 7.20 (t, 1H), 7.54 (d, 2H), 7.66 (s, 1H), 7.73 (d, 1H), 8.42 (s, 1H), 12.89 (sbr, 1H): LC/MS: m/z 380 (M+1), 382 (M+3).

### Example 90

### 2-(2-Chloro-phenylamino)-5-(6-methoxy-pyridin-3-ylmethylene)-thiazol-4-one

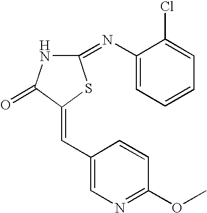

¹H NMR (DMSO-d₆) δ 3.88 (s, 3H), 6.92 (d, 1H), 71-6 (d, 1H), 7.21 (t, 1H), 7.37 (t, 1H), 7.54 (d, 1H), 7.67 (s, 1H), 7.75 (dd, 1H), 8.43 (dd, 1H), 12.66 (sbr, 1H): LC/MS: m/z 346 (M+1), 348 (M+3).

### Example 91

### 2-(2-Chloro-5-trifluoromethyl-phenylamino)-5-quinolin-6-ylmethylene-thiazol-4-one

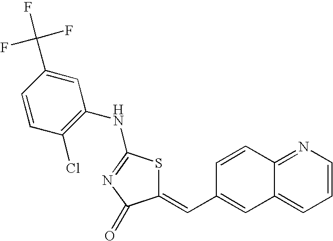

¹H NMR (DMSO-d₆) δ 7.56 (m, 3H), 7.87 (mbr, 3H), 8.06 (d, 1H), 81-6 (s, 1H), 8.44 (d, 1H), 8.92 (m, 1H), 12.89 (sbr, 1H): LC/MS m/z 434 (M+1), 436 (M+3).

### Example 92

### 2-(2-Bromo-phenylamino)-5-(4-hydroxy-3-methoxy-benzylidene)-thiazol-4-one

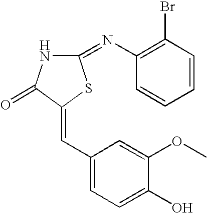

¹H NMR (DMSO-d₆) δ 3.75 (s, 3H), 6.87 (m, 2H), 7.10 (m, 2H), 7.13 (s, 1H), 7.38 (m, 1H), 7.53 (s, 1H), 7.67 (d, 1H), 9.77 (sbr, 1H): LC/MS m/z 405 (M), 407 (M+2).

### Example 93

### 5-(2,3-Dihydro-benzofuran-5-ylmethylene)-2-(2-methoxy-phenylamino)-thiazol-4-one

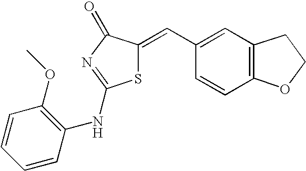

¹H NMR (DMSO-d₆) δ 3.19 (t, 2H), 3.76 (s, 3H), 4.57 (t, 2H), 6.87 (t, 1H), 6.98 (mbr, 2H), 7.09 (d, 1H), 7.19 (m, 1H), 7.26 (d, 1H), 7.35 (s, 1H), 7.56 (s, 1H), 11.0 (sbr, 1H) LC/MS m/z 353 (M+1).

### Example 94

### 2-(2-Nitro-phenylamino)-5-quinolin-6-ylmethylene-thiazol-4-one

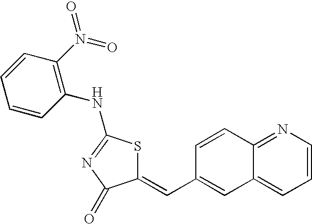

¹H NMR (DMSO-d₆) δ 7.24 (sbr, 1H), 7.40 (sbr, 1H), 7.55 (dd, 1H), 7.63 (mbr, 2H), 7.89 (m, 2H), 8.06 (d, 1H), 8.11 (d, 1H), 8.43 (d, 1H), 8.91 (dd, 1H): LC/MS m/z 377 (M+1).

### Example 95

### 2-(2-Bromo-phenylamino)-5-(3,4-diamino-benzylidene)-thiazol-4-one

### Scheme C

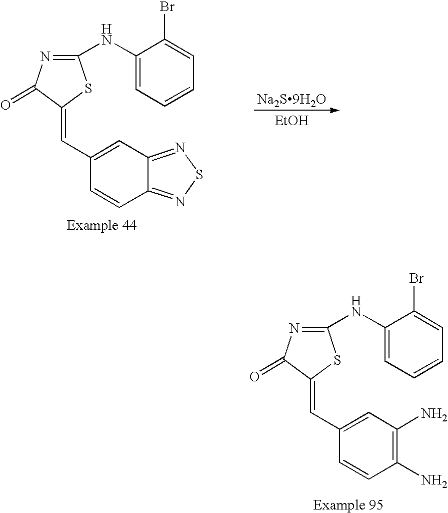

A mixture of the product of compound of Example 44 (380 mg) and Na₂S-9H₂O (600 mg) in ethanol was irradiated by a microwave reactor at 120 C° for 5 hours. The mixture was poured onto aq.NH₄Cl and the formed orange precipitate was filtrated. Washing with H₂O and subsequent desiccation gave compound the title compound.

¹H NMR (DMSO-d₆) δ 4.68 (sbr, 2H), 5.30 (s, 2H), 6.44-6.55 (m, 3H), 7.04 (m, 2H), 7.29 (s, 1H), 7.33 (t, 1H), 7.61 (d, 1H): LC/MS: m/z 389 (M), 391 (M+2).

### Example 96

### 5-[2-(2-Chloro-phenylimino)-4-oxo-thiazolidin-5-yl idenemethyl]-1-methyl-1H-pyridin-2-one

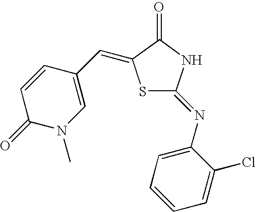

¹H NMR (400 MHz, DMSO-d₆) ppm 3.45 (s, 3H), 6.48 (d, 1H, J=9.6 Hz), 7.13 (d, 1H, J=7.8 Hz), 7.19 (m, 1H), 7.36 (m, 1H), 7.43 (s, 1H), 7.47 (dd, 1H, J=2.6, 9.6 Hz), 7.53 (d, 1H, J=8.1 Hz), 8.23 (d, 1H, J=2.8 Hz), 12.15 (br, 1H). LC/MS: m/z 346 (M+1)+, 344 (M−1)−.

### Synthesis of Compounds of Example 97-99

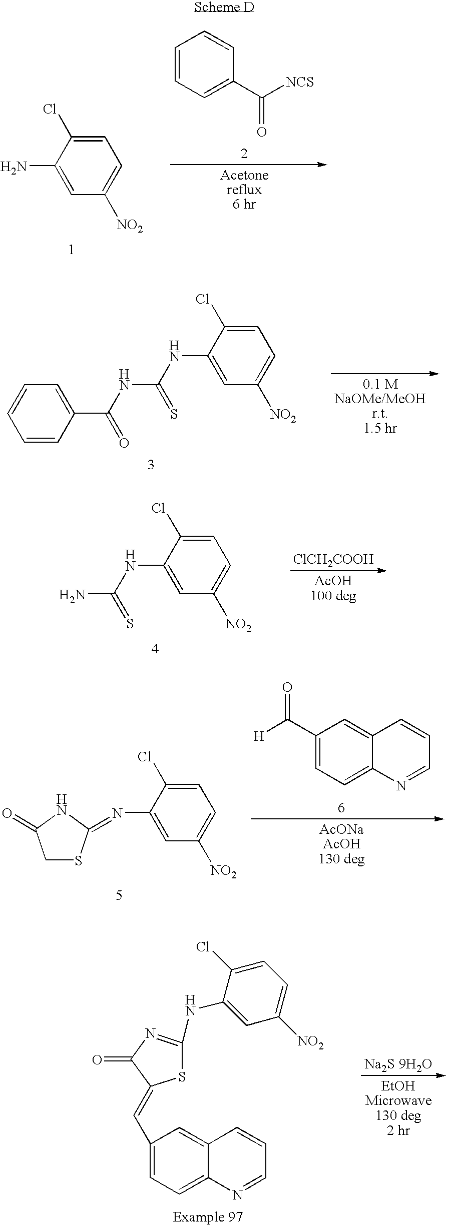

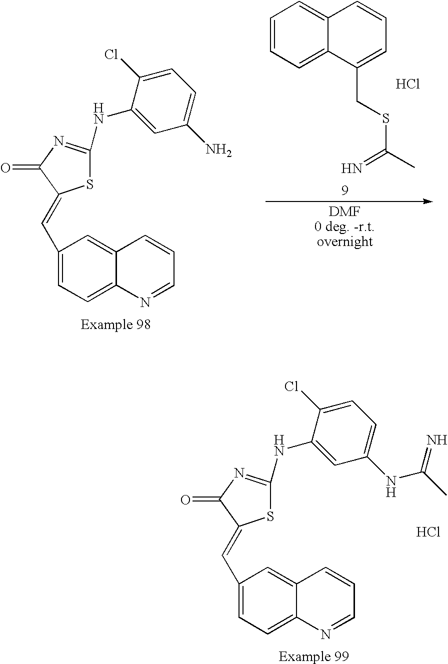

### Example 97

### 2-(2-Chloro-5-nitro-phenylamino)-5-quinolin-6-ylmethylene-thiazol-4-one

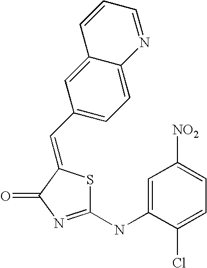

A mixture of 2-chloro-5-nitroaniline 1 (5.2 g, 30 mmol) and benzoyl isothiacyanate 2 (5.4 g, 33 mmol) in 40 ml of acetone was refluxed for 6 hours and then cooled and left to stand at room temperature. The separated crystalline solid was collected by filtration and washed with acetone and dried in vacuo to give benzoyl thiourea 3 (9.4 g, 28 mmol). 9.0 g (26.8 mmol) of Benzoyl thiourea 3 was treated with 600 ml of 0.1 M sodium methoxide solution. The yellow-orange solution formed was left to stand at room temperature overnight, then neutralized with methanolic hydrogen chloride to pH 7. The resulting solution was treated with 100 ml of water and the mixture was concentrated to 200 ml by vacuum distillation. The separated yellow crystalline solid was collected by filtration. After recrystallization from water-acetone(2:1) 2.77 g (11.9 mmol) of thiourea 4 was obtained. A mixture of 4 (2.3 g, 10 mmol) and ClCH₂CO₂H (1.1 g) in AcOH (20 mL) was heated at 100 C° for overnight. The mixture was poured onto water and the formed solid was isolated by filtration. It was washed with water to give thiazolidinone 5 (1.65 g, 6.1 mmol). A mixture of 5 (272 mg, 1.0 mmol), aldehyde 6 (157 mg, 1.0 mmol) and AcONa (246 mg, 3.0 mmol) in AcOH (10 mL) was heated to reflux at 130 C.° for 10 days. Generated solid was collected by filtration and washed with AcOH and water, followed by desiccation in vacuo to afford the title product (328 mg, 0.80 mmol). 1H NMR (DMSO-d₆) [symbol] 12.95 (s, 1H), 8.94 (m, 1H), 8.45 (d, 1H), 8.16 (s, 1H), 8.09-8.04 (m, 3H), 7.90 (s, 1H), 7.90-7.85 (m, 2H), 7.57 (dd, 1H): LC/MS: m/z 411 (M+1)

### Example 98

### 2-(5-Amino-2-chloro-phenylamino)-5-quinolin-6-ylmethylene-thiazol-4-one

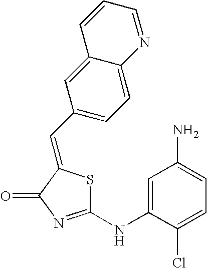

A mixture of 2-(2-chloro-5-nitro-phenylamino)-5-quinolin-6-ylmethylene-thiazol-4-one (100 mg, 0.42 mmol) and sodium sulfide nonahydrate (350 mg, 1-66 mmol) in 4 ml of EtOH was heated by microwave (SmithSynthesizer-Personal Chemistry) at 130□ for 2 hours. The reaction mixture was cooled to room temperature and concentrated by vacuum distillation, then water was added and neutralized with aqueous ammonium chloride. Generated solid was collected by filtration and washed with water, followed by desiccation in vacuo to afford the title product (33 mg, 0.086 mmol). 1H NMR (DMSO-d6) [symbol] 12.63 (s, 1H) 8.94 (m, 1H) 8.47 (d, 1H) 4.16 (s, 1H) 8.10 (d, 1H) 7.87 (dd, 1H) 7.84 (s, 1H) 7.56 (dd, 1H) 7.12 (d, 1H) 6.40 (d, 1H) 6.38 (s, 1H) 5.37 (s, 2H).

: LC/MS: m/z 381 (M+1)

### Example 99

### N-[4-Chloro-3-(4-oxo-5-quinolin-6-ylmethylene-4,5-dihydro-thiazol-2-ylamino)-phenyl]-acetamidine hydrochloride

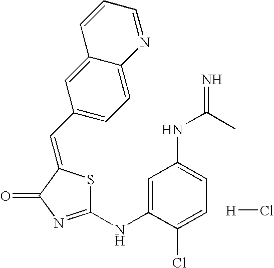

To a stirred, cooled(0 deg.) solution of 2-(5-amino-2-chloro-phenylamino)-5-quinolin-6-ylmethylene-thiazol-4-one (39.7 mg, 0.1 mmol) in DMF (1 ml) was added thioacetimidate hydrochloride 9 (28 mg, 0.11 mmol). The mixture was warmed to room temperature and stirred for over night. DMF was removed by nitrogen gas blowing and resulting oil was dissolved with methanol. Insoluble solid was collected by filtration and washed with methanol, followed by desiccation in vacuo to afford the title (14 mg, 0.031 mmol).

¹H NMR (DMSO-d₆) [symbol] 12.87 (s, 1H) 11.29 (s, 1H) 9.55 (s, 1H) 8.95 (m, 1H) 8.67 (s, 1H) 8.44 (d, 1H) 8.18 (s, 1H) 8.09 (s, 1H) 7.89 (s, 1H) 7.87 (dd, 1H) 7.74 (d, 1H) 7.60 (dd, 1H) 7.22-7.15 (m, 2H) 2.31 (s, 3H). LC/MS: m/z 422 (M+1)

Note: Thioacetimidate hydrochloride 9 was made according to a procedure in Tetrahedron Letters, Vol. 38, No2, pp. 179-182, 1997.

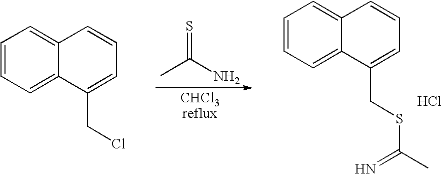

Compounds of Examples 100 to 109 were made analogous to a process describe in Scheme B and Example 62.

### Example 100

### 4-{[4-oxo-5-(6-quinolinylmethylidene)-4,5-dihydro-1,3-thiazol-2-yl]amino}benzamide

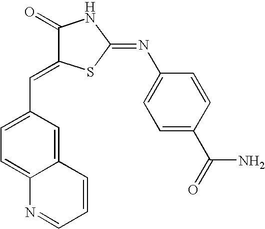

1H NMR (400 MHz, DMSO-d₆) ppm 7.11 (d, 1H), 7.48 (d, 1H), 7.55 (m, 1H), 7.82-8.04 (m, 4H), 8.10 (d, 1H), 8.15 (s, 1H), 8.45 (d, 1H), 8.83 (d, 1H), 11.86 (s, 1H). LC/MS: m/z 375 (M+1)+.

### Example 101

### 3-{[4-oxo-5-(6-quinolinylmethylidene)-4,5-dihydro-1,3-thiazol-2-yl]amino}benzenesulfonamide

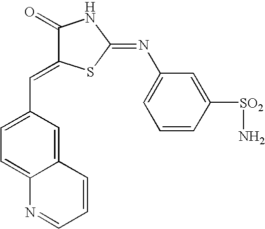

1H NMR (400 MHz, DMSO-d₆) ppm 7.40-8.70 (m, 5H), 7.82 (s, 1H), 7.98 (d, 1H), 8.07 (d, 1H), 8.17 (s, 1H), 8.45 (d, 1H), 8.95 (d, 1H). LC/MS: m/z 411 (M+1)⁺.

### Example 102

### 4-{[4-oxo-5-(6-quinolinylmethylidene)-4,5-dihydro-1,3-thiazol-2-yl]amino}-N-2-pyridinylbenzenesulfonamide

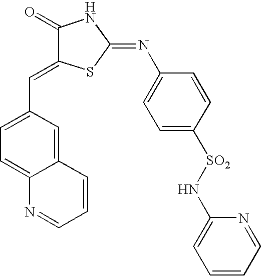

1H NMR (400 MHz, DMSO-d₆) ppm 5.95 (s, 1H), 6.54 (d, 2H), 6.89 (m, 1H), 7.05 (d, 1H), 7.20 (m, 1H), 7.50 (d, 2H), 7.63 (m, 1H), 7.7-8.2 (m, 4H), 8.45 (m, 1H)ₘ, 8.95 (m, 1H). LC/MS: m/z 488 (M+1)+.

### Example 103

### 2-({4-[(4-methyl-1-piperazinyl)methyl]phenyl}amino)-5-(6-quinolinylmethylidene)-1,3-thiazol-4 (5H)-one

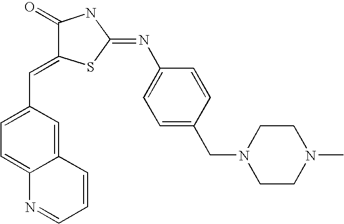

1H NMR (400 MHz, DMSO-d₆) ppm 2.38 (s, 3H), 2.65 (m, 4H), 2.86 (m, 4H), 3.68 (s, 2H), 7.06 (d, 1H), 7.38 (d, 2H), 7.60 (m, 1H), 7.76 (d, 1H), 7.80 (s, 1H), 7.91 (s, 1H), 8.00 (d, 2H) 8.25 (m, 1H), 8.45 (m, 1H), 8.95 (m, 1H). LC/MS: m/z 444 (M+1)+.

### Example 104

### 2-({4-[(methylsulfonyl)methyl]phenyl}amino)-5-(6-quinolinylmethylidene)-1,3-thiazol-4 (5H)-one

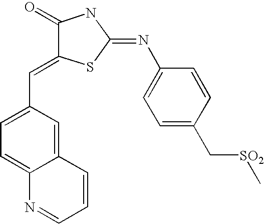

1H NMR (400 MHz, DMSO-d₆) ppm 2.92 (s, 3H), 4.52 (s, 2H), 6.53 (d, 1H), 7.01 (d, 1H), 7.10 (m, 1H), 7.48 (d, 2H), 7.62 (m, 1H), 7.83 (d, 2H), 7.95 (s, 1H), 8.47 (d, 1H), 8.95 (m, 1H), 11.80 (s, 1H). LC/MS: m/z 424 (M+1)+.

### Example 105

### 2-({3-[(methylsulfonyl)methyl]phenyl}amino)-5-(6-quinolinylmethylidene)-1,3-thiazol-4(5H)-one

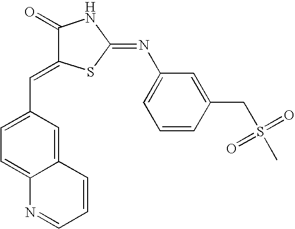

1H NMR (400 MHz, DMSO-d₆) ppm 2.96 (s, 3H), 4.58 (s, 2H), 6.53 (d, 1H), 7.12 (m, 1H), 7.35 (d, 1H), 7.50 (m, 1H), 7.61 (m, 1H), 7.85 (s, 1H), 7.95 (d, 1H), 8.05 (s, 1H), 8.11 (s, 1H), 8.47 (d, 1H), 8.95 (m, 1H), 11.80 (s, 1H). LC/MS: m/z 424 (M+1)+.

### Example 106

### 2-{[4-(4-methyl-1-piperazinyl)phenyl]amino}-5-(6-quinolinylmethylidene)-1,3-thiazol-4(5H)-one

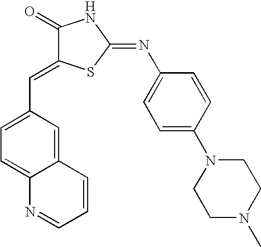

1H NMR (400 MHz, DMSO-d₆) ppm 2.25 (d, 4H), 3.28 (d, 4H), 3.35 (s, 3H), 6.98 (d, 2H), 7.00 (d, 1H), 7.53 (m, 1H), 7.62 (d, 2H), 7.85 (s, 1H), 7.95 (d, 1H), 8.21 (s, 1H), 8.47 (d, 1H), 8.95 (m, 1H), 11.80 (s, 1H). LC/MS: m/z 430 (M+1)+.

HPLC retention times in the following Examples were taken by the method: Agilent Eclipse ODS 4.6×250 mm, 1.5 mL/min, 5-95% Water/ACN in 10 min.

### Example 107

### 2-{[2-(3-chlorophenyl)ethyl]amino}-5-(6-quinolinylmethylidene)-1,3-thiazol-4(5H)-one

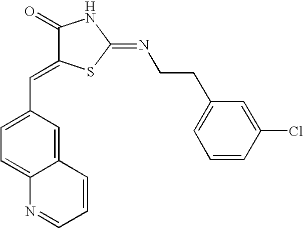

HPLC Rt=4.74 min. LC/MS: m/z 394 (M−1)+, 396 (M+1)+.

### Example 108

### 4-(2-{[4-oxo-5-(6-quinolinylmethylidene)-4,5-dihydro-1,3-thiazol-2-yl]amino}ethyl)benzenesulfonamide

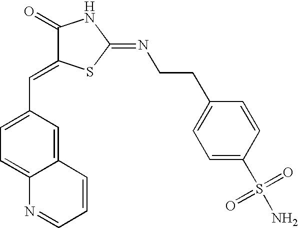

HPLC Rt=3.49 min. LC/MS: m/z 439 (M+1)+.

### Example 109

### 3-{[4-oxo-5-(6-quinolinylmethylidene)-4,5-dihydro-1,3-thiazol-2-yl]amino}benzamide

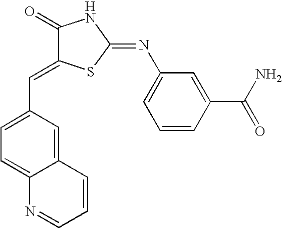

HPLC Rt=3.57 min. LC/MS: m/z 375 (M+1)+.

Compounds in Examples 110-115 were made according to Scheme A with modification that a thiourea of formula III was made according to a method described by Walczynski K. et al. in II Farmaco 55 (2000) 569-574 (Scheme E), or by Rasmussen, F. J. et. al. in Synthesis 1988, 456-459.

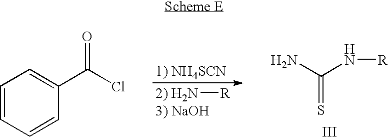

### Example 110

### 2-[(2,6-Difluoro-phenylamino)-methylene]-5-quinolin-6-ylmethylene-thiazolidin-4-one

Benzoyl chloride (5.44 g, 38 mmol) was added dropwise to a solution of ammonium thiocyanate (2.55 g, 42.6 mmol) in acetone. Solution was refluxed for 10 minutes at which time a solution of 2,6-difluoro-aniline (5.0 g, 38.8 mmol) in acetone was added dropwise and the solution refluxed for approximately 5 minutes. The solution was then poured into 500 mL of water and a resulting solid precipitated out. The separated crystalline solid was collected by filtration and then heated in a NaOH solution (3 g in 50 mL H₂O). The solution was acidified with conc. HCl, then made slightly basic using conc. NH₄OH. Crystalline solid was seen and collected to obtain (2,6-difluoro-phenyl)-thiourea. A mixture of the thiourea (5.7 g, 30.3 mmol), AcONa (2.43 g) and ClCH₂CO₂H (2.86 g) in AcOH (20 mL) was heated to reflux at 130 C° for four hours. The mixture was poured onto water and the formed solid was isolated by filtration. It was washed with water to give the desired thiazolidinone (a compound of formula IV in which R is 2,6-difluorophenyl) (6.75 g, 29.6 mmol). A mixture of the thiazolidinone (200 mg, 0.8 mmol), 6-formyl quinoline (137 mg, 0.8 mmol) and AcONa (211 mg, 2.4 mmol) in AcOH (10 mL) was heated to reflux at 130 C° for 2 days. Water was added to the solution and generated a solid that was collected by filtration and washed with water, followed by desiccation in vacuo to afford the title compound as a yellow solid. ES (+/−) MS m/e=368.0 (M+H). HPLC (rt) −4.53m

### Example 111

### 2-[(2,6-Difluoro-phenylamino)-methylene]-5-quinolin-6-ylmethylene-thiazolidin-4-one

Prepared according to the procedure as in Example 110 above, except using 4-chloro-2-methyl-phenyl)-thiourea (2.00 g, 9.98 mmol; commercially available) as the appropriate thiourea. Title compound was a yellow solid. ES (+/−) MS m/e=379.8 (M+H). HPLC (rt) −5.19 m

### Example 112

### [2,4-Dichloro-5-(4-oxo-5-quinolin-6-ylmethylene-thiazolidin-2-ylideneamino)-phenoxy]-acetic acid

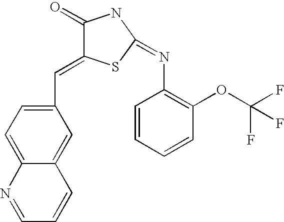

Prepared according to Example 110, except using commercially available benzoyl isothiocyanate instead of generating in situ. A solid precipitate formed and was collected to afford the title compound as a brownish yellow solid. ES (+/−) MS m/e=416.0 (M+H). HPLC (rt) −5.24m.

### Example 113

### 2-[2,4-Dichloro-5-(2-methoxy-ethoxy)-phenylimino]-5-quinolin-6-ylmethylene-thiazolidin-4-one

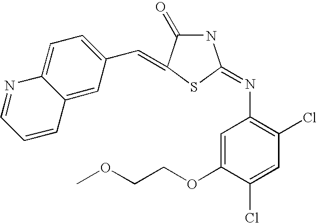

Prepared according to Example 110 above, except using commercially available benzoyl isothiocyanate instead of generating it in situ. A solid precipitate formed and was collected to afford the title compound as a yellow solid. ES (+/−) MS m/e=474.2 (M+H). HPLC (rt) −5.54m

### Example 114

### 4-Chloro-3-(4-oxo-5-quinolin-6-ylmethylene-thiazolidin-2-ylideneamino)-benzoic acid

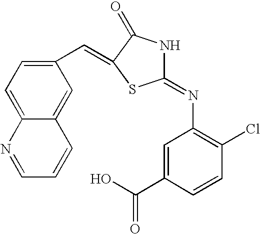

Prepared according to Example 110, except using commercially available benzoyl isothiocyanate instead of generating it in situ. The thiourea did not crystallize so the solution was removed in vacuo and used in thiazolidinone step. The sodium salts were filtered away during this step while the HOAc solution was still hot. The final step yielded a solid precipitate which was collected to afford the title compound as a white solid. ES (+/−) MS m/e=410.2 (M+H). HPLC (rt) −4.12m

### Example 115

### [2,4-Dichloro-5-(4-oxo-5-quinolin-6-ylmethylene-thiazolidin-2-ylideneamino)-phenoxy]-acetic acid

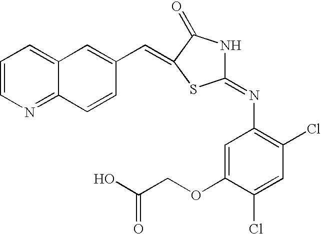

Prepared according to Example 110, except using commercially available benzoyl isothiocyanate instead of generating it in situ. The initial aniline, (5-Amino-2,4-dichloro-phenoxy)-acetic acid ethyl ester, was hydrolyzed to an acid by stirring with NaOH. The thiourea did not crystallize so the solution was removed in vacuo and used in thiazolidinone step. The sodium salts were filtered away during this step while the HOAc solution was still hot. The final step yielded a solid precipitate which was collected to afford the title compound as a white solid. ES (+/−) MS m/e=474.0 (M+H). HPLC (rt) −4.73m

While the suitable embodiments of the invention are illustrated by the above, it is to be understood that the invention is not limited to the precise instructions herein disclosed and that the right to all modifications coming within the scope of the following claims is reserved.

What is claimed is:

1-13. (canceled)

14. A method of inhibiting one or more phosphatoinositides 3-kinases (PI3Ks) in a mammal; comprising administering to the mammal a therapeutically effective amount of a compound of Formula (I): 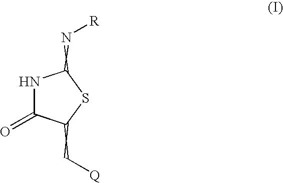
in which
R is C₃₋₆ cycloalkyl or naphtyl; or
R is 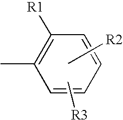
in which R1 is hydrogen, halogen, —C₁₋₆alkyl, —SC₁₋₆alkyl, —OC₁₋₆alkyl, —NO₂, —S(═O)—C₁₋₆alkyl, —OH, —CF₃, —CN, —CO₂H, —OCF₃, or —CO₂C₁₋₆alkyl;
and R2 and R3 are independently hydrogen, halogen, —C₁₋₆ alkyl, —SC₁₋₆alkyl, —OC₁₋₆alkyl, —NO₂, —S(═O)—C₁₋₆alkyl, —OH, —CF₃, —CN, —CO₂H, —CO₂C₁₋₆alkyl, —CONH₂, —NH₂, —OCH₂(C═O)OH, —OCH₂CH₂OCH₃, —SO₂NH₂, —CH₂SO₂CH₃, —NH(C═NH)CH₃; or R2 and R3 can independently be a radical of the formula 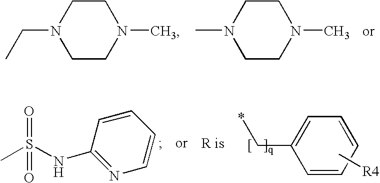
in which q is one or two; R4 is hydrogen, halogen, or —SO₂NH₂; or
R is —(CH₂)ₙ—NR^{k}R^{l} in which n is 2 or 3, and R^{k} and R^{l} are independently —C₁₋₆alkyl; or —NR^{k}R^{l} together form 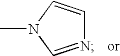
R is 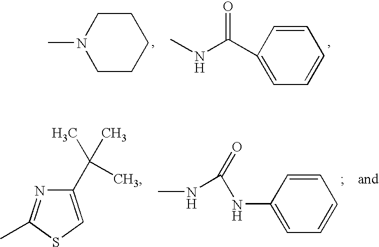
Q is 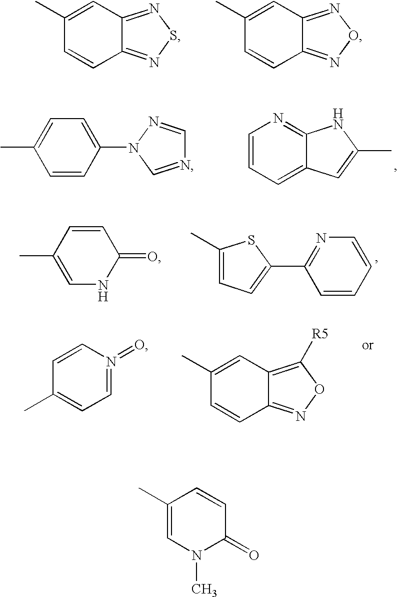
in which R5 is hydrogen, phenyl optionally substituted with up to three C₁₋₆ alkyl or halogen, or C₁₋₆ alkyl or
Q is 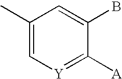
in which Y is CH; and A and B together are a part of 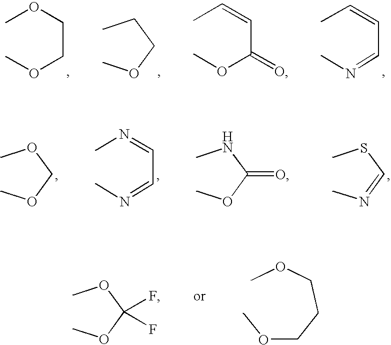
provided that ortho position to Y is N or O; or
Q is 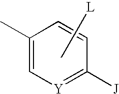
in which Y is N or CH; J is hydrogen, NH₂, OH or —OC₁₋₆alkyl; and L is hydrogen, NH₂, halogen, —NO₂, or —OC₁₋₆alkyl,
and/or a pharmaceutically acceptable salt, hydrate, solvate or pro-drug thereof.

15. A method of treating one or more disease state selected from the group consisting of: autoimmune disorders, inflammatory diseases, cardiovascular diseases, neurodegenerative diseases, allergy, asthma, pancreatitis, multiorgan failure, kidney diseases, platelet aggregation, cancer, sperm motility, transplantation rejection, graft rejection and lung injuries, in a mammal, which method comprises administering to such mammal, a therapeutically effective amount of a compound according to claim 14.

16. A method of treating cancer comprises co-administration a compound of formula I and/or a pharmaceutically acceptable salt, hydrate, solvate or pro-drug thereof and at least one anti-neoplastic agent, such as one selected from the group consisting of anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine kinase angiogenesis inhibitors, immunotherapeutic agents, proapoptotic agents, and cell cycle signaling inhibitors.

17. The method of claim 15, wherein the disease state is selected from the group consisting of: multiple sclerosis, psoriasis, rheumatoid arthritis, systemic lupus erythematosis, inflammatory bowel disease, lung inflammation, thrombosis, brain infection/inflammation, meningitis and encephalitis.

18. The method of claim 15, wherein the disease state is selected from the group consisting of: Alzheimer's disease, Huntington's disease, CNS trauma, stroke and ischemic conditions.

19. The method of claim 15, wherein the disease state is selected from the group consisting of: atherosclerosis, heart hypertrophy, cardiac myocyte dysfunction, elevated blood pressure and vasoconstriction.

20. The method of claim 15, wherein the disease state is selected from the group consisting of: chronic obstructive pulmonary disease, anaphylactic shock fibrosis, psoriasis, allergic diseases, asthma, stroke, ischemia-reperfusion, platelets aggregation/activation, skeletal muscle atrophy/hypertrophy, leukocyte recruitment in cancer tissue, antiogenesis, invasion metastasis, melanoma, Karposi's sarcoma, acute and chronic bacterial and virual infections, sepsis, transplantation rejection, graft rejection, glomerulo sclerosis, glomerulo nephritis, progressive renal fibrosis, endothelial and epithelial injuries in the lung, and lung airways inflammation.

21. The method of claim 15 wherein the disease is cancer.

22. The method of claim 15 wherein the disease is selected from a group consisting of: ovarian cancer, pancreatic cancer, breast cancer, prostate cancer and leukemia.

23. The method of claim 15 wherein the mammal is human.

24. The method of claim 14, wherein said PI3 kinase is a PI3α.

25. The method of claim 14, wherein said PI3 kinase is a PI3γ.

26. The method of claim 14, wherein said compound is selected from:
2-(2-Chloro-5-fluoro-phenylimino)-5-(2,3-dihydro-benzo[1-6]dioxin-6-ylmethylene)-thiazolidin-4-one;
2-(2-Chloro-phenylimino)-5-(2-oxo-2H-chromen-6-ylmethylene)-thiazolidin-4-one;
2-(2-Chloro-phenylimino)-5-(2-oxo-2H-chromen-6-ylmethylene)-thiazolidin-4-one;
2-(2-Chloro-phenylimino)-5-(2-oxo-2H-chromen-6-ylmethylene)-thiazolidin-4-one;
5-(2,3-Dihydro-benzofuran-5-ylmethylene)-2-(2,4,6-trimethyl-phenylimino)-thiazolidin-4-one;
2-Cyclohexylimino-5-(2,3-dihydro-benzo[1-6]dioxin-6-ylmethylene)-thiazolidin-4-one;
2-Cyclohexylimino-5-(2,3-dihydro-benzofuran-5-ylmethylene)-thiazolidin-4-one;
5-(2,3-Dihydro-benzofuran-5-ylmethylene)-2-o-tolylimino-thiazolidin-4-one;
5-(2,3-Dihydro-benzo[1-6]dioxin-6-ylmethylene)-2-o-tolylimino-thiazolidin-4-one;
5-[2-(2-Chloro-phenylimino)-4-oxo-thiazolidin-5-ylidenemethyl]-3H-benzooxazol-2-one;
2-(2-Trifluoromethyl-phenylimino)-5-(2,3-dihydro-benzofuran-5-ylmethylene)-thiazolidin-4-one;
2-(2-Bromo-phenylimino)-5-(2,3-dihydro-benzofuran-5-ylmethylene)-thiazolidin-4-one;
2-(2,6-Dichloro-phenylimino)-5-(2,3-dihydro-benzofuran-5-ylmethylene)-thiazolidin-4-one;
5-(2,3-Dihydro-benzofuran-5-ylmethylene)-2-(2-methylsulfanyl-phenylimino)-thiazolidin-4-one;
5-(2,3-Dihydro-benzofuran-5-ylmethylene)-2-(2-fluoro-phenylimino)-thiazolidin-4-one;
2-(2-Methylsulfanyl-phenylimino)-5-(quinolin-6-ylmethylene)-thiazolidin-4-one;
2-(2-Bromo-phenylimino)-5-(quinolin-6-ylmethylene)-thiazolidin-4-one;
2-(2,3-Dimethyl-phenylimino)-5-(quinolin-6-ylmethylene)-thiazolidin-4-one;
2-(Naphthalen-1-ylimino)-5-(quinolin-6-ylmethylene)-thiazolidin-4-one;
5-(Quinolin-6-ylmethylene)-2-(2-trifluoromethyl-phenylimino)-thiazolidin-4-one;
2-(2-Chloro-5-trifluoromethyl-phenylimino)-5-(quinolin-6-ylmethylene)-thiazolidin-4-one;
2-(2,6-Dichloro-phenylimino)-5-8quinolin-6-ylmethylene)-thiazolidin-4-one;
2-(2-Bromo-phenylimino)-5-(2,3-dihydro-benzo[1-6]dioxin-6-ylmethylene)-thiazolidin-4-one;
2-(2-Chloro-phenylimino)-5-(quinoxalin-6-ylmethylene)-thiazolidin-4-one;
2-(2,6-Dichloro-phenylimino)-5-(2,3-dihydro-benzo[1-6]dioxin-6-ylmethylene)-thiazolidin-4-one;
5-(2,3-Dihydro-benzo[1-6]dioxin-6-ylmethylene)-2-(2-nitro-phenylimino)-thiazolidin-4-one;
5-(2,3-Dihydro-benzofuran-5-ylmethylene)-2-(2-nitro-phenylimino)-thiazolidin-4-one;
2-(2-Chloro-4-fluoro-5-methyl-phenylimino)-5-(2,3-dihydro-benzofuran-5-ylmethylene)-thiazolidin-4-one;
3-Chloro-4-[5-(2,3-dihydro-benzofuran-5-ylmethylene)-4-oxo-thiazolidin-2-ylideneamino]-benzoic acid methyl ester;
2-(2-Chloro-5-fluoro-phenylimino)-5-(2,3-dihydro-benzofuran-5-ylmethylene)-thiazolidin-4-one;
2-(2-Chloro-4-trifluoromethyl-phenylimino)-5-(2,3-dihydro-benzofuran-5-ylmethylene)-thiazolidin-4-one;
2-(4-Bromo-2-chloro-phenylimino)-5-(2,3-dihydro-benzofuran-5-ylmethylene)-thiazolidin-4-one;
5-(2,3-Dihydro-benzofuran-5-ylmethylene)-2-(2-methanesulfinyl-phenylimino)-thiazolidin-4-one;
3-Chloro-4-[5-(2,3-dihydro-benzofuran-5-ylmethylene)-4-oxo-thiazolidin-2-ylideneamino]-benzoic acid;
5-[2-(2-Chloro-phenylimino)-4-oxo-thiazolidin-5-ylidenemethyl]-1H-pyridin-2-one;
2-(2-Methylsulfanyl-phenylimino)-5-(quinolin-6-ylmethylene)-thiazolidin-4-one;
2-(2-Chloro-4-fluoro-5-methyl-phenylimino)-5-(quinolin-6-ylmethylene)-thiazolidin-4-one;
2-(2-Chloro-5-fluoro-phenylimino)-5-(quinolin-6-ylmethylene)-thiazolidin-4-one;
2-(2-Chloro-5-fluoro-phenylimino)-5-(2,3-dihydro-benzo[1-6]dioxin-6-ylmethylene)-thiazolidin-4-one;
2-(2-Chloro-4-trifluoromethyl-phenylimino)-5-quinolin-6-ylmethylene-thiazolidin-4-one;
5-(Benzothiazol-6-ylmethylene)-2-(2-chloro-phenylimino)-thiazolidin-4-one;
5-(Benzo[1,2,5]thiadiazol-5-ylmethylene)-2-(2-bromo-phenylimino)-thiazolidin-4-one;
5-(Benzol[1,2,5]thiadiazol-5-ylmethylene)-2-(2-chloro-5-fluoro-phenylimino)-thiazolidin-4-one;
5-(Benzothiazol-6-ylmethylene)-2-(2,6-dichloro-phenylimino)-thiazolidin-4-one;
2-(2-Chloro-phenylimino)-5-(4-hydroxy-3-nitro-benzylidene)-thiazolidin-4-one;
2-(2-Chloro-phenylimino)-5-(4-hydroxy-3-methoxy-benzylidene)-thiazolidin-4-one;
2-(2-Chloro-phenylimino)-5-(4-hydroxy-benzylidene)-thiazolidin-4-one;
2-(2-Chloro-phenylimino)-5-(4-methoxy-benzylidene)-thiazolidin-4-one;
5-(3-Chloro-4-hydroxy-benzylidene)-2-(2-chloro-phenylimino)-thiazolidin-4-one;
2-(2-Chloro-phenylimino)-5-(3-fluoro-4-methoxy-benzylidene)-thiazolidin-4-one;
2-(2,6-Dichloro-phenylimino)-5-(3-fluoro-4-hydroxy-benzylidene)-thiazolidin-4-one;
2-(2-Chloro-phenylimino)-5-(3-fluoro-4-hydroxy-benzylidene)-thiazolidin-4-one;
2-(2-Chloro-5-fluoro-phenylimino)-5-(3-fluoro-4-hydroxy-benzylidene)-thiazolidin-4-one;
5-(3-Fluoro-4-hydroxy-benzylidene)-2-o-tolylimino-thiazolidin-4-one;
2-(2-Chloro-phenylimino)-5-quinolin-6-ylmethylene-thiazolidin-4-one;
5-Quinolin-6-ylmethylene-2-(2,4,6-trimethyl-phenylimino)-thiazolidin-4-one;
5-Quinolin-6-ylmethylene-2-o-tolylimino-thiazolidin-4-one;
2-(2-Methoxy-phenylimino)-5-quinolin-6-ylmethylene-thiazolidin-4-one;
5-(2,3-Dihydro-benzofuran-5-ylmethylene)-2-(2-dimethylamino-ethylamino)-thiazol-4-one;
Benzoic acid N′-(4-oxo-5-quinolin-6-ylmethylene-4,5-dihydro-thiazol-2-yl)-hydrazide;
2-(2-Dimethylamino-ethylimino)-5-quinolin-6-ylmethylene-thiazolidin-4-one;
5-(2,3-Dihydro-benzofuran-5-ylmethylene)-2-(piperidin-1-ylamino)-thiazol-4-one;
2-Benzylamino-5-(2,3-dihydro-benzofuran-5-ylmethylene)-thiazol-4-one;
2-(4-tert-Butyl-thiazol-2-ylamino)-5-(2,3-dihydro-benzofuran-5-ylmethylene)-thiazol-4-one;
4-{[5-(2,3-Dihydro-benzofuran-5-ylmethylene)-4-oxo-4,5-dihydro-thiazol-2-ylamino]-methyl}-benzenesulfonamide;
5-(2,3-Dihydro-benzofuran-5-ylmethylene)-2-(3-dimethylamino-propylamino)-thiazol-4-one;
5-(2,3-Dihydro-benzofuran-5-ylmethylene)-2-(3-imidazol-1-yl-propylamino)-thiazol-4-one;
Phenyl-carbamic acid N′-[5-(2,3-dihydro-benzofuran-5-ylmethylene)-4-oxo-4,5-dihydro-thiazol-2-yl]-hydrazide;
Benzoic acid N′-[5-(2,3-dihydro-benzofuran-5-ylmethylene)-4-oxo-4,5-dihydro-thiazol-2-yl]-hydrazide;
5-Benzo[1,2,5]thiadiazol-5-ylmethylene-2-(2,3,4-trifluoro-phenylamino)-thiazol-4-one;
5-Benzo[1, 2, 5]oxadiazol-5-ylmethylene-2-(2-nitro-phenylamino)-thiazol-4-one
2-(2,6-Dichloro-phenylamino)-5-(4-[1,2,4]triazol-1-yl-benzylidene)-thiazol-4-one;
2-(2,6-Dichloro-phenylamino)-5-(1H-pyrrolo[2,3-b]pyridin-2-ylmethylene)-thiazol-4-one;
5-Benzo[1,2,5]thiadiazol-5-ylmethylene-2-(2,6-dichloro-phenylamino)-thiazol-4-one;
5-[2-(2-Methoxy-6-methyl-phenylamino)-4-oxo-4H-thiazol-5-ylidenemethyl]-1H-pyridin-2-one;
5-Benzo[1,2,5]thiadiazol-5-ylmethylene-2-(2-nitro-phenylamino)-thiazol-4-one;
2-(2-Bromo-6-fluoro-phenylamino)-5-quinolin-6-ylmethylene-thiazol-4-one;
2-(2-Methoxy-6-methyl-phenylamino)-5-quinolin-6-ylmethylene-thiazol-4-one;
5-Quinolin-6-ylmethylene-2-(2,3,4-trifluoro-phenylamino)-thiazol-4-one;
2-(2,6-Dichloro-phenylamino)-5-(2-oxo-2H-chromen-6-ylmethylene)-thiazol-4-one;
2-(2-Bromo-phenylamino)-5-(5-pyridin-2-yl-thiophen-2-ylmethylene)-thiazol-4-one;
2-(2-Bromo-phenylamino)-5-(1-oxy-pyridin-4-ylmethylene)-thiazol-4-one;
2-(2-Bromo-phenylamino)-5-(3-p-tolyl-benzo[c]isoxazol-5-ylmethylene)-thiazol-4-one;
2-(2-Bromo-phenylamino)-5-(3,4-dihydro-2H-benzo[b][1-6]dioxepin-7-ylmethylene)-thiazol-4-one;
5-Benzo[1, 2, 5]oxadiazol-5-ylmethylene-2-(2-bromo-phenylamino)-thiazol-4-one;
2-(2,6-Dichloro-phenylamino)-5-(2-methoxy-pyridin-3-ylmethylene)-thiazol-4-one;
2-(2-Chloro-phenylamino)-5-(6-methoxy-pyridin-3-ylmethylene)-thiazol-4-one;
2-(2-Chloro-5-trifluoromethyl-phenylamino)-5-quinolin-6-ylmethylene-thiazol-4-one;
2-(2-Bromo-phenylamino)-5-(4-hydroxy-3-methoxy-benzylidene)-thiazol-4-one;
5-(2,3-Dihydro-benzofuran-5-ylmethylene)-2-(2-methoxy-phenylamino)-thiazol-4-one;
2-(2-Nitro-phenylamino)-5-quinolin-6-ylmethylene-thiazol-4-one;
2-(2-Bromo-phenylamino)-5-(3,4-diamino-benzylidene)-thiazol-4-one;
5-[2-(2-Chloro-phenylimino)-4-oxo-thiazolidin-5-ylidenemethyl]-1-methyl-1H-pyridin-2-one;
2-(2-Chloro-5-nitro-phenylamino)-5-quinolin-6-ylmethylene-thiazol-4-one;
2-(5-Amino-2-chloro-phenylamino)-5-quinolin-6-ylmethylene-thiazol-4-one;
N-[4-Chloro-3-(4-oxo-5-quinolin-6-ylmethylene-4,5-dihydro-thiazol-2-ylamino)-phenyl]-acetamidine hydrochloride;
4-{[4-oxo-5-(6-quinolinylmethylidene)-4,5-dihydro-1,3-thiazol-2-yl]amino}benzamide;
3-{[4-oxo-5-(6-quinolinylmethylidene)-4,5-dihydro-1,3-thiazol-2-yl]amino}benzenesulfonamide;
4-{[4-oxo-5-(6-quinolinylmethylidene)-4,5-dihydro-1,3-thiazol-2-yl]amino}-N-2-pyridinylbenzenesulfonamide;
2-({4-[(4-methyl-1-piperazinyl)methyl]phenyl}amino)-5-(6-quinolinylmethylidene)-1,3-thiazol-4(5H)-one;
2-({4-[(methylsulfonyl)methyl]phenyl}amino)-5-(6-quinolinylmethylidene)-1,3-thiazol-4(5H)-one;
2-({3-[(methylsulfonyl)methyl]phenyl}amino)-5-(6-quinolinylmethylidene)-1,3-thiazol-4(5H)-one;
2-{[4-(4-methyl-1-piperazinyl)phenyl]amino}-5-(6-quinolinylmethylidene)-1,3-thiazol-4(5H)-one;
2-{[2-(3-chlorophenyl)ethyl]amino}-5-(6-quinolinylmethylidene)-1,3-thiazol-4(5H)-one;
4-(2-{[4-oxo-5-(6-quinolinylmethylidene)-4,5-dihydro-1,3-thiazol-2-yl]amino}ethyl)benzenesulfonamide;
3-{[4-oxo-5-(6-quinolinylmethylidene)-4,5-dihydro-1,3-thiazol-2-yl]amino}benzamide;
2-[(2,6-Difluoro-phenylamino)-methylene]-5-quinolin-6-ylmethylene-thiazolidin-4-one;
2-[(2,6-Difluoro-phenylamino)-methylene]-5-quinolin-6-ylmethylene-thiazolidin-4-one;
[2,4-Dichloro-5-(4-oxo-5-quinolin-6-ylmethylene-thiazolidin-2-ylideneamino)-phenoxy]-acetic acid;
2-[2,4-Dichloro-5-(2-methoxy-ethoxy)-phenylimino]-5-quinolin-6-ylmethylene-thiazolidin-4-one;
4-Chloro-3-(4-oxo-5-quinolin-6-ylmethylene-thiazolidin-2-ylideneamino)-benzoic acid;
[2,4-Dichloro-5-(4-oxo-5-quinolin-6-ylmethylene-thiazolidin-2-ylideneamino)-phenoxy]-acetic acid;
and/or a pharmaceutically acceptable salt, hydrate, solvate or pro-drug thereof.

27. A method of claim one wherein the compound of formula (I), and/or a pharmaceutically acceptable salt, hydrate, solvate or pro-drug thereof, is administered in a pharmaceutical composition.
